(19) United States
(12) Patent Application Publication
(10) Pub. No.: US 2004/0132133 A1
(43) Pub. Date: Jul. 8, 2004

(54) Methods and compositions for the production, identification and purification of fusion proteins

(75) Inventor: Robert P. Bennett, Encinitas, CA (US)

Correspondence Address: STERNE, KESSLER, GOLDSTEIN & FOX PLLC, 1100 NEW YORK AVENUE, N.W., WASHINGTON, DC, 20005, US

(73) Assignee: Invitrogen Corporation

(21) Appl. No.: 10/612,410

(22) Filed: Jul. 3, 2003

Related U.S. Application Data
(60) Provisional application No. 60/417,172, filed on Oct. 10, 2002.
(60) Provisional application No. 60/396,627, filed on Jul. 19, 2002.
(60) Provisional application No. 60/393,756, filed on Jul. 8, 2002.

(51) Int. Cl. C12Q 1/68; C07H 21/04; C07K 14/47

(52) U.S. Cl. 435/69.1; 435/320.1; 435/325; 530/350; 536/23.5; 435/6

(57) ABSTRACT

The present invention provides compositions and methods for producing fusion proteins that comprise an amino acid sequence tag. The amino acid sequence tag may be an amino acid sequence that is capable of being post-translationally modified; for example, the amino acid sequence may be an amino acid sequence that is capable of being biotinylated. The amino acid sequence tag may also be an amino acid sequence that is recognized by an antibody (or fragment thereof) or other specific interacting reagent. The invention includes isolated nucleic acid molecules comprising one or more nucleic acid sequences which encode an amino acid sequence tag. The nucleic acid molecules of the invention may also comprise one or more recombination sites and/or one or more topoisomerase recognition sites and/or one or more topoisomerases. The nucleic acid molecules of the invention can be used in recombinational cloning and/or topoisomerase-mediated cloning methods in order to produce polynucleotide constructs which encode fusion proteins that comprise an amino acid sequence tag. Also provided are host cells, kits and compositions comprising the nucleic acid molecules of the invention.

### CROSS REFERENCE TO RELATED APPLICATIONS

The present application claims the benefit of U.S. Provisional Patent Application No. 60/393,756, filed Jul. 8, 2002, U.S. Provisional Patent Application No. 60/396,627, filed Jul. 19, 2002, and U.S. Provisional Patent Application No. 60/417,172, filed Oct. 10, 2002. The contents of the aforesaid applications are relied upon and incorporated by reference in their entirety.

### BACKGROUND OF THE INVENTION

1. Field of the Invention

The present invention relates to compositions and methods for producing fusion proteins. More specifically, the invention relates to compositions and methods for producing fusion proteins that comprise an amino acid sequence tag. Exemplary amino acid sequence tags include amino acid sequences that are capable of being post-translationally modified, and amino acid sequences that are capable of being recognized by an antibody (or fragment thereof) or other specific binding reagent.

The invention relates to nucleic acid molecules that can be used in recombinational cloning methods and/or topoisomerase-mediated cloning methods to produce polynucleotide constructs that encode fusion proteins, e.g., fusion proteins that comprise one or more amino acid sequence tags. The invention also relates to methods for producing fusion proteins in a variety of prokaryotic and eukaryotic cell types. The invention also relates to methods for identifying and purifying fusion proteins by utilizing, e.g., binding molecules and compositions that bind specifically to the fusion protein.

2. Related Art

Many areas of biotechnology and molecular biology rely on the production and purification of recombinant proteins. When recombinant proteins are produced in vivo they are generally produced in addition to a wide variety of endogenous proteins and other macromolecules in a host cell. Various strategies are employed to isolate and/or identify recombinant proteins from the cellular milieu. One strategy is to produce a fusion protein which comprises the protein of interest joined to an amino acid sequence tag.

When a fusion protein is produced that comprises a tag that is capable of being post-translationally modified, the post-translational modification can be exploited to isolate or identify the fusion protein, especially when (a) very few or no endogenous proteins or molecules contain the same post-translational modification in the host cell, and (b) a molecule is available which is capable of physically interacting with the post-translationally modified protein.

One particular post-translational modification that has been used to isolate and/or identify recombinant fusion proteins is biotinylation. For instance, a fusion protein can be produced which comprises a protein of interest joined to an amino acid sequence to which a biotin moiety can be covalently bound. The biotinylation reaction will occur in vivo, i.e., in the host cell. The biotinylated fusion protein can then be isolated from the endogenous components of the host cell by providing a molecule that interacts specifically with the biotin moiety. Usually, the biotin-interacting molecule will be bound to a bead or other solid support which can be easily separated from the rest of the cellular components.

Amino acid sequences which are capable of being biotinylated include, for example, a domain the 1.3S subunit of Propionibacterium shermanii transcarboxylase (PSTCD) that is naturally biotinylated at lysine 89 of the domain. (Cronan, J. E., J. Biol. Chem. 265:10327-10333 (1990); Murtif, V. L., et al., Proc. Natl. Acad. Sci. USA 82:5617-5621 (1985)). Another example is a 72 amino acid peptide derived from the C-terminus (amino acids 524-595) of the Klebsiella pneumoniae oxalacetate decarboxylase α subunit. (Schwarz, E. et al., J. Biol. Chem. 263:9640-9645 (1988)). Fusion proteins containing biotinylation domains have been shown to be biotinylated by endogenous biotinylation components in bacteria, yeast and mammalian cells. (Cronan, J. E., J. Biol. Chem. 265:10327-10333 (1990); Jank, M. M. et al., Protein Expr. Purif. 17:123-127 (1999); Parrott, M. B. and Barry, M. A., Biochem. Biophys. Res. Comm. 281:993-1000 (2001); Parrott, M. B. and Barry, M. A., Molecular Therapy 1:96-104 (2000); U.S. Pat. No. 5,252,466 and references cited therein).

Avidin has been shown to interact very strongly with biotin. The non-covalent interaction between avidin and biotin represents one of the strongest and most specific interactions commonly used in molecular biology. The interaction between avidin and biotin is estimated to have an affinity coefficient of 10⁻¹⁴ to 10⁻¹⁵, which is several orders of magnitude greater than a typical antibody-antigen interaction. (Rosano, C. et al., Biomol. Eng. 16:5-12 (1999); Green, N. M., Methods Enzymol. 184:51-67 (1990); Airenne, K. J. et al., Protein Expr. Purif. 17:139-145 (1999); Wilchek, M. and Bayer, E. A., Methods Enzymol. 184:5-13 (1990)). Avidin analogs, including streptavidin are also available for specifically interacting with biotin.

As an alternative to producing a protein or polypeptide that is capable of being post-translationally modified, it is sometimes useful to produce a fusion protein that comprises an amino acid sequence that is identifiable by particular reagents, including, e.g., antibodies (or fragments thereof) or other binding compounds that can recognize certain polypeptides or amino acid sequences.

In order to produce a recombinant fusion protein that comprises a particular amino acid sequence tag, a nucleic acid molecule must first be constructed which encodes the desired fusion protein. The construction of the recombinant nucleic acid molecule will generally involve the attachment of at least two individual nucleotide sequences: (1) a sequence encoding the protein of interest, and (2) a sequence encoding an amino acid sequence tag.

Multiple nucleic acid sequences can be joined using conventional in vitro cloning methods which employ restriction endonucleases and DNA ligation enzymes. More rapid and efficient methods are available, however, which involve site-specific recombination and/or topoisomerase-mediated joining of nucleic acid sequences. Recombinational and topoisomerase-mediated cloning methods have been described in detail elsewhere. (Hartley, J. L., et al., Genome Res. 10:1788-1795 (2000); Shuman, S., J. Biol. Chem. 269:32678-32684 (1994); Shuman, S., Proc. Natl. Acad. Sci. USA 88:10104-10108 (1991); U.S. Pat. Nos. 5,851,808, 5,888,732, 6,143,557, 6,171,861, 6,270,969, 6,277,608 and 6,410,317; and commonly owned, co-pending U.S. patent application Ser. No. 10/005,876 (filed Dec. 7, 2001)).

Briefly, recombinational cloning, specifically the Gateway™ Cloning System (available from Invitrogen Corporation), utilizes vectors that contain at least one and preferably at least two different site-specific recombination sites based on the bacteriophage lambda system (e. g., att1 and att2) that are mutated from the wild type (att0) sites. Each mutated site has a unique specificity for its cognate partner att site of the same type (for example attB1 with attP1, or attL1 with attR1) and will not cross-react with recombination sites of the other mutant type or with the wild-type att0 site. Nucleic acid fragments flanked by recombination sites are cloned and subcloned using the Gateway™ system by replacing a selectable marker (for example, ccdb) flanked by att sites on the recipient plasmid molecule, sometimes termed the Destination Vector. Desired clones are then selected by transformation of a ccdB sensitive host strain and positive selection for a marker on the recipient molecule. Similar strategies for negative selection (e.g., use of toxic genes) can be used in other organisms such as thymidine kinase (TK) in mammals and insects. Other recombinational cloning systems are available such as, e.g., Echo™ (Invitrogen Corporation) and Creator (Clontech).

Topoisomerase cloning can be used to generate a double-stranded recombinant nucleic acid molecule covalently linked in one strand. This method can be performed by contacting a first nucleic acid molecule which has a site-specific topoisomerase recognition site (e.g., a type IA or a type II topoisomerase recognition site), or a cleavage product thereof, at a 5′ or 3′ terminus, with a second (or other) nucleic acid molecule, and optionally, a topoisomerase (e.g., a type IA, type IB, and/or type II topoisomerase), such that the second nucleotide sequence can be covalently attached to the first nucleotide sequence. Topoisomerase cloning can also be used to generate a double-stranded recombinant nucleic acid molecule covalently linked in both strands. This method can be performed, for example, by contacting a first nucleic acid molecule having a first end and a second end, wherein, at the first end or second end or both, the first nucleic acid molecule has a topoisomerase recognition site (or cleavage product thereof) at or near the 3′ terminus; at least a second nucleic acid molecule having a first end and a second end, wherein, at the first end or second end or both, the at least second double stranded nucleotide sequence has a topoisomerase recognition site (or cleavage product thereof) at or near a 3′ terminus; and at least one site specific topoisomerase (e.g., a type IA and/or a type IB topoisomerase), under conditions such that all components are in contact and the topoisomerase can effect its activity. A covalently linked double-stranded recombinant nucleic acid by this method is characterized, in part, in that it does not contain a nick in either strand at the position where the nucleic acid molecules are joined. The method may be performed by contacting a first nucleic acid molecule and a second (or other) nucleic acid molecule, each of which has a topoisomerase recognition site, or a cleavage product thereof, at the 3′ termini or at the 5′ termini of two ends to be covalently linked. Alternatively, the method can be performed by contacting a first nucleic acid molecule having a topoisomerase recognition site, or cleavage product thereof, at the 5′ terminus and the 3′ terminus of at least one end, and a second (or other) nucleic acid molecule having a 3′ hydroxyl group and a 5′ hydroxyl group at the end to be linked to the end of the first nucleic acid molecule containing the recognition sites. Topoisomease cloning methods can be performed using any number of nucleic acid molecules having various combinations of termini and ends.

Cloning schemes are also available which use both recombinational cloning and topoisomerase cloning methods. Such methods may involve first joining two nucleic acid sequences using recombinational cloning to create a product nucleic acid molecule, followed by joining the product nucleic acid molecule to another nucleic acid molecule using topoisomerase cloning. Conversely, two nucleic acid molecules may joined, first, by using topoisomerase cloning to create a product nucleic acid molecule, followed by joining the product nucleic acid molecule to another nucleic acid molecule using recombinational cloning.

Recombinational cloning methods, topoisomerase cloning methods, and combinations thereof, heretofore have not been described in the art for producing nucleic acid constructs that encode fusion proteins that comprise one or more amino acid sequence tags. Accordingly, a need exists in the art for rapid and efficient compositions and methods that enable the production of nucleic acid molecules which encode fusion proteins.

### BRIEF SUMMARY OF THE INVENTION

The present invention satisfies the aforementioned need in the art by providing compositions and methods for producing fusion proteins which comprise one or more amino acid sequences of interest and one or more amino acid sequence tags. An “amino acid sequence tag,” as used herein, includes, e.g., amino acid sequences that are capable of being post-translationally modified, and/or amino acid sequences that are capable of being recognized by an antibody (or fragment thereof) or other specific binding reagent.

The invention includes isolated nucleic acid molecules comprising one or more nucleic acid sequences which encode an amino acid sequence tag. The isolated nucleic acid molecules of the invention may further comprise one or more recombination sites. Alternatively or additionally, the isolated nucleic acid molecules of the invention may further comprise one or more topoisomerase recognition sites and/or one or more topoisomerases. Thus, in certain embodiments, the invention includes isolated nucleic acid molecules comprising: (a) one or more recombination sites; (b) one or more topoisomerase recognition sites and/or one or more topoisomerases; and (c) one or more nucleic acid sequences which encode an amino acid sequence tag.

In addition to the aforementioned elements, the nucleic acid molecules of the invention may further comprise additional elements. Exemplary additional elements that may be included within the nucleic acid molecules of the invention include, e.g., one or more promoters, one or more operators, one or more enhancers, one or more ribosome binding sites, one or more initiation codons, one or more nucleic acid sequences that encodes an amino acid sequence that is capable of being cleaved by one or more proteases, one or more nucleic acid sequences of interest (e.g., one or more nucleic acid sequences that encode one or more proteins or polypeptides of interest), one or more polyadenylation signals and/or one or more transcription termination regions. As understood by those skilled in the art, other elements may be included within the nucleic acid molecules of the invention depending on the circumstances under which the nucleic acids may be used.

In a preferred embodiment, the elements of the isolated nucleic acid molecules of the invention are arranged relative to one another such that a nucleic acid sequence of interest can be attached to the nucleic acid molecules of the invention, thereby producing a polynucleotide construct that encodes a fusion protein, the fusion protein comprising: (i) an amino acid sequence tag; and (ii) the amino acid sequence encoded by said nucleic acid sequence of interest. The fusion protein may be, e.g., an N-terminal fusion protein (e.g., wherein an amino acid sequence tag is covalently attached at or near the N-terminus of the amino acid sequence encoded by said nucleic acid sequence of interest). The fusion protein may also be, e.g., a C-terminal fusion protein (e.g., wherein an amino acid sequence tag is covalently attached at or near the C-terminus of the amino acid sequence encoded by said nucleic acid sequence of interest). The fusion protein may also be, e.g., an N-terminal and C-terminal fusion protein (e.g., wherein an amino acid sequence tag is covalently attached at or near the N-terminus of the amino acid sequence encoded by said nucleic acid sequence of interest and an amino acid sequence tag is covalently attached at or near the C-terminus of the amino acid sequence encoded by said nucleic acid sequence of interest).

The invention also includes nucleic acid molecules that are created following the attachment of a nucleic acid sequence of interest to a nucleic acid molecule comprising: (a) a nucleic acid sequence that encodes an amino acid sequence tag; and/or (b) one or more recombination sites; and/or (c) one or more topoisomerase recognition sites and/or one or more topoisomerases.

In order to produce a polynucleotide sequence that encodes a fusion protein that comprises one or more amino acid sequence tags, a nucleic acid sequence of interest may, for example, be inserted at or within 20 nucleotides of said one or more recombination sites. The nucleic acid sequence may also be inserted at or within 20 nucleotides of said one or more topoisomerase recognition sites and/or at or within 20 nucleotides of the position of said one or more topoisomerases in order to produce a polynucleotide sequence that encodes a fusion protein that comprises an amino acid sequence tag.

The nucleic acid molecules of the invention may further comprise a nucleic acid sequence that encodes an amino acid sequence that is capable of being cleaved by one or more proteases. The position of such a nucleic acid sequence, relative to the other elements of the nucleic acid molecules of the invention, will be such that, a nucleic acid sequence of interest can be attached to the nucleic acid molecules of the invention, thereby producing a polynucleotide construct that encodes a fusion protein, the fusion protein comprising: (i) said amino acid sequence that is capable of being cleaved by one or more proteases, flanked on one side by (ii) the amino acid sequence tag, and on the other side by (iii) the amino acid sequence encoded by the amino acid sequence of interest.

In certain embodiments, the nucleic acid sequence that encodes an amino acid sequence tag may be, e.g., a nucleic acid sequence that encodes an amino acid sequence that is capable of being post-translationally modified. For example, the nucleic acid sequence may be a nucleic acid sequence which encodes an amino acid sequence that is capable of being post-translationally modified by, e.g., biotinylation, attachment of 4-phosphopanthetheine, attachment of lipoic acid, attachment of flavins, etc. In a preferred embodiment, the amino acid sequence is capable of being biotinylated. An exemplary nucleic acid sequence that encodes a protein or polypeptide having an amino acid sequence that is capable of being biotinylated is an amino acid sequence which encodes a portion of the C-terminus of the Klebsiella pneumoniae oxalacetate decarboxylase α subunit, e.g., an amino acid sequence known as the Biotag™.

In certain other embodiments, the nucleic acid sequence that encodes an amino acid sequence tag may be, e.g., a nucleic acid sequence which encodes an amino acid sequence that is capable of being recognized by an antibody (or fragment thereof) or other specific binding reagent. Such amino acid sequences are known in the art and include, e.g., a 6-Histidine tag, an epitope tag (e.g., an amino acid sequence recognized by a specific antibody (or fragment thereof) such as, e.g., the FLAG tag, the Myc tag, the HA tag, etc.) Thus, the nucleic acid molecules of the invention can, in some embodiments, be used to produce fusion proteins comprising: (i) an amino acid sequence which encodes an amino acid sequence that is capable of being recognized by a specific antibody (or fragment thereof) or other compound or reagent, and (ii) an amino acid sequence encoded by a nucleotide sequence of interest.

The invention also includes methods for producing polynucleotide constructs that encode fusion proteins that comprise one or more amino acid sequence tags. In certain embodiments, the invention generally includes methods of attaching a first nucleic acid molecule (e.g., a nucleic acid molecule which has a nucleotide sequence which encodes a particular protein or polypeptide of interest) to a second nucleic acid molecule which comprises one or more nucleic acid sequence tags. The attachment of the first nucleic acid molecule to the second nucleic acid molecule may be accomplished by, e.g., recombination (e.g., recombinational cloning) and/or by topoisomerase-mediated cloning. The attachment of the first nucleic acid molecule to the second nucleic acid molecule will preferably result in a product polynucleotide construct which encodes a fusion protein, said fusion protein comprising: (i) the amino acid sequence tag; and (ii) the amino acid sequence encoded by the nucleotide sequence of the first nucleic acid molecule.

The invention also includes methods of producing fusion proteins that comprise one or more amino acid sequence tags. Also included are methods for producing fusion proteins that can be purified, concentrated or otherwise identified. The methods, according to this aspect of the invention, may comprise: (a) obtaining a host cell comprising a polynucleotide construct that encodes a fusion protein that comprises one or more amino acid sequence tags, said polynucleotide construct produced according to a method of the invention; and (b) culturing said host cell under conditions wherein said fusion protein is produced by said host cell. The methods of the invention may further comprise culturing said host cell under conditions wherein said fusion protein is post-translationally modified in said host cell. In other embodiments of this aspect of the invention, the methods further comprise: (a) causing said fusion protein to be released from said host cell or treating said host cell such that said fusion protein is released from said host cell; and (b) contacting said fusion protein with a detecting composition comprising a molecule that is capable of interacting specifically with said fusion protein.

In certain exemplary embodiments, said fusion protein is a fusion protein that has been post-translationally modified, e.g., a biotinylated fusion protein, and said detecting composition comprises avidin, streptavidin, or analogs and derivatives thereof.

The invention further comprises vectors comprising the nucleic acid molecules of the invention, host cells comprising the nucleic acid and/or vectors of the invention, and kits comprising the nucleic acid molecules, vectors, and/or host cells of the invention.

### BRIEF DESCRIPTION OF THE DRAWINGS

FIG. 1 is a map which shows the general characteristics of pET104-DEST.

FIGS. 2A-2C show the nucleotide sequence of pET104-DEST (SEQ ID NO:1).

FIG. 3 is a map which shows the general characteristics of pET104/GW/lacZ.

FIG. 4 is a map which shows the general characteristics of pET104/D-TOPO.

FIGS. 5A-5B show the nucleotide sequence of pET104/D-TOPO (SEQ ID NO:2).

FIG. 6 is a map which shows the general characteristics of pET104/D/lacZ.

FIG. 7 is a map which shows the general characteristics of pcDNA6/Biotag™-DEST.

FIGS. 8A-8B show the nucleotide sequence of pcDNA6/Biotag™-DEST (SEQ ID NO:3).

FIG. 9 is a map which shows the general characteristics of pcDNA6/Biotag™-GW/lacZ.

FIG. 10 is a map which shows the general characteristics of pcDNA6/Biotag™/D-TOPO.

FIGS. 11A-11B show the nucleotide sequence of pcDNA6/Biotag™/D-TOPO (SEQ ID NO:4).

FIG. 12 is a map which shows the general characteristics of pcDNA6/Biotag™/lacZ.

FIG. 13 is a map which shows the general characteristics of pMT/Biotag™-DE ST.

FIGS. 14A-14B show the nucleotide sequence of pMT/Biotag™-DEST (SEQ ID NO:5).

FIG. 15 is a map which shows the general characteristics of pMT/Biotag™/GW-lacZ.

FIG. 16 is a depiction of the recombination region of the expression clone resulting from pET104-DEST x entry clone, showing the nucleotide sequence of the recombination region (SEQ ID NO:25) and the amino acid sequence encoded therefrom (SEQ ID NO:26).

FIG. 17 is a schematic representation of the mechanism by which TOPO cloning is accomplished.

FIG. 18 is a flow-chart describing the general steps required for cloning and expressing a blunt-end PCR product using pET104/D-TOPO.

FIG. 19 is a depiction of a region of the pET104/D-TOPO vector surrounding the Biotag™, showing the nucleotide sequence of the region (SEQ ID NO:27) and the amino acid sequence encoded therefrom (SEQ ID NO:28).

FIG. 20 is a depiction of the recombination region of the expression clone resulting from pcDNA6/Biotag™-DEST x entry clone, showing the nucleotide sequence of the recombination region (SEQ ID NO:29) and the amino acid sequence encoded therefrom (SEQ ID NO:30).

FIG. 21 is a flow-chart describing the general steps required for cloning and expressing a blunt-end PCR product using pcDNA6/Biotag™/D-TOPO.

FIG. 22 is a depiction of a region of the pcDNA6/Biotag™/D-TOPO vector surrounding the Biotag™, showing the nucleotide sequence of the region (SEQ ID NO:31) and the amino acid sequence encoded therefrom (SEQ ID NO:32).

FIG. 23 is a depiction of the recombination region of the expression clone resulting from pMT/Biotag™-DEST x entry clone, showing the nucleotide sequence of the recombination region (SEQ ID NO:33) and the amino acid sequence encoded therefrom (SEQ ID NO:34).

FIG. 24 is a map which shows the general characteristics of pCoHygro.

FIG. 25 is a map which shows the general characteristics of pCoBlast.

### DETAILED DESCRIPTION OF THE INVENTION

The present invention relates generally to compositions and methods for producing nucleic acid molecules which encode fusion proteins, e.g., fusion proteins that comprise one or more amino acid sequence tags. The invention also relates to methods for producing, purifying, concentrating and isolating fusion proteins using the compositions and methods described herein.

The invention relates to nucleic acid molecules comprising: (a) one or more recombination sites; and (b) one or more nucleic acid sequences which encode one or more amino acid sequence tags.

The invention also relates to isolated nucleic acid molecules comprising: (a) one or more topoisomerase recognition sites and/or one or more topoisomerases; and (b) one or more nucleic acid sequences which encode one or more amino acid sequence tags.

The invention also relates to isolated nucleic acid molecules comprising: (a) one or more recombination sites; (b) one or more topoisomerase recognition sites and/or one or more topoisomerases; and (c) one or more nucleic acid sequences which encode one or more amino acid sequence tags.

The nucleic acid molecules of the invention may be circular molecules, or they may be linear molecules.

As used herein, a nucleotide is a base-sugar-phosphate combination. Nucleotides are monomeric units of a nucleic acid molecule (DNA and RNA). The term nucleotide includes ribonucleoside triphosphates ATP, UTP, CTG, GTP and deoxyribonucleoside triphosphates such as dATP, dCTP, dITP, dUTP, dGTP, dTTP, or derivatives thereof. Such derivatives include, for example, [(S]dATP, 7-deaza-dGTP and 7-deaza-dATP. The term nucleotide as used herein also refers to dideoxyribonucleoside triphosphates (ddNTPs) and their derivatives. Illustrated examples of dideoxyribonucleoside triphosphates include, but are not limited to, ddATP, ddCTP, ddGTP, ddITP, and ddTTP. According to the present invention, a “nucleotide” may be unlabeled or detectably labeled by well known techniques. Detectable labels include, for example, radioactive isotopes, fluorescent labels, chemiluminescent labels, bioluminescent labels and enzyme labels.

As used herein, a nucleic acid molecule is a sequence of contiguous nucleotides (riboNTPs, dNTPs or ddNTPs, or combinations thereof) of any length which may encode a full-length polypeptide or a fragment of any length thereof, or which may be non-coding. As used herein, the terms “nucleic acid molecule” and “polynucleotide” and “polynucleotide construct” may be used interchangeably.

Polymerases for use in the invention include but are not limited to polymerases (DNA and RNA polymerases), and reverse transcriptases. DNA polymerases include, but are not limited to, Thermus thermophilus (Tth) DNA polymerase, Thermus aquaticus (Taq) DNA polymerase, Thermotoga neopolitana (Tne) DNA polymerase, Thermotoga maritima (Tma) DNA polymerase, Thermococcus litoralis (Tli or VENT™) DNA polymerase, Pyrococcus furiosus (Pfu) DNA polymerase, DEEPVENT™ DNA polymerase, Pyrococcus woosii (Pwo) DNA polymerase, Pyrococcus sp KOD2 (KOD) DNA polymerase, Bacillus sterothermophilus (Bst) DNA polymerase, Bacillus caldophilus (Bca) DNA polymerase, Sulfolobus acidocaldarius (Sac) DNA polymerase, Thermoplasma acidophilum (Tac) DNA polymerase, Thermus flavus (Tfl/Tub) DNA polymerase, Thermus ruber (Tru) DNA polymerase, Thermus brockianus (DYNAZYME™) DNA polymerase, Methanobacterium thermoautotrophicum (Mth) DNA polymerase, mycobacterium DNA polymerase (Mtb, Mlep), E. coli pol I DNA polymerase, T5 DNA polymerase, T7 DNA polymerase, and generally pol I type DNA polymerases and mutants, variants and derivatives thereof. RNA polymerases such as T3, T5, T7 and SP6 and mutants, variants and derivatives thereof may also be used in accordance with the invention.

The nucleic acid polymerases used in the present invention may be mesophilic or thermophilic, and are preferably thermophilic. Preferred mesophilic DNA polymerases include Pol I family of DNA polymerases (and their respective Klenow fragments) any of which may be isolated from organism such as E. coli, H. influenzae, D. radiodurans, H. pylori, C. aurantiacus, R. prowazekii, T.pallidum, Synechocystis sp., B. subtilis, L. lactis, S. pneumoniae, M. tuberculosis, M. leprae, M. smegmatis, Bacteriophage L5, phi-C31, T7, T3, T5, SP01, SP02, mitochondrial from S. cerevisiae MIP-1, and eukaryotic C. elegans, and D. melanogaster (Astatke, M. et al., 1998, J. Mol. Biol. 278, 147-165), pol III type DNA polymerase isolated from any sources, and mutants, derivatives or variants thereof, and the like. Preferred thermostable DNA polymerases that may be used in the methods and compositions of the invention include Taq, Tne, Tma, Pfu, KOD, Tfl, Tth, Stoffel fragment, VENT™ and DEEPVENT™ DNA polymerases, and mutants, variants and derivatives thereof (U.S. Pat. Nos. 5,436,149; 4,889,818; 4,965,188; 5,079,352; 5,614,365; 5,374,553; 5,270,179; 5,047,342; 5,512,462; WO 92/06188; WO 92/06200; WO 96/10640; WO 97/09451; Barnes, W. M., Gene 112:29-35 (1992); Lawyer, F. C., et al., PCR Meth. Appl. 2:275-287 (1993); Flaman, J.-M, et al., Nucl. Acids Res. 22(15):3259-3260 (1994)).

Reverse transcriptases for use in this invention include any enzyme having reverse transcriptase activity. Such enzymes include, but are not limited to, retroviral reverse transcriptase, retrotransposon reverse transcriptase, hepatitis B reverse transcriptase, cauliflower mosaic virus reverse transcriptase, bacterial reverse transcriptase, Tth DNA polymerase, Taq DNA polymerase (Saiki, R. K., et al., Science 239:487-491 (1988); U.S. Pat. Nos. 4,889,818 and 4,965,188), Tne DNA polymerase (WO 96/10640 and WO 97/09451), Tma DNA polymerase (U.S. Pat. No. 5,374,553) and mutants, variants or derivatives thereof (see, e.g., WO 97/09451 and WO 98/47912). Preferred enzymes for use in the invention include those that have reduced, substantially reduced or eliminated RNase H activity. By an enzyme “substantially reduced in RNase H activity” is meant that the enzyme has less than about 20%, more preferably less than about 15%, 10% or 5%, and most preferably less than about 2%, of the RNase H activity of the corresponding wildtype or RNase H⁺ enzyme such as wildtype Moloney Murine Leukemia Virus (M-MLV), Avian Myeloblastosis Virus (AMV) or Rous Sarcoma Virus (RSV) reverse transcriptases. The RNase H activity of any enzyme may be determined by a variety of assays, such as those described, for example, in U.S. Pat. No. 5,244,797, in Kotewicz, M. L., et al., Nucl. Acids Res. 16:265 (1988) and in Gerard, G. F., et al., FOCUS 14(5):91 (1992), the disclosures of all of which are fully incorporated herein by reference. Particularly preferred polypeptides for use in the invention include, but are not limited to, M-MLV H⁻ reverse transcriptase, RSV H⁻ reverse transcriptase, AMV H⁻ reverse transcriptase, RAV (rous-associated virus) H⁻ reverse transcriptase, MAV (myeloblastosis-associated virus) H⁻ reverse transcriptase and HIV H⁻ reverse transcriptase. (See U.S. Pat. No. 5,244,797 and WO 98/47912). It will be understood by one of ordinary skill, however, that any enzyme capable of producing a DNA molecule from a ribonucleic acid molecule (i.e., having reverse transcriptase activity) may be equivalently used in the compositions, methods and kits of the invention.

As used herein, a polypeptide is a sequence of contiguous amino acids, of any length. As used herein, the terms “peptide,” “oligopeptide,” or “protein” may be used interchangeably with the term “polypeptide.

As used herein, the term “amino acid sequence tag” is intended to mean any amino acid sequence that can be attached to, connected to, or linked to a heterologous amino acid sequence (e.g., an amino acid sequence of interest) and that can be used to identify, purify, concentrate or isolate said heterologous amino acid sequence. The attachment of the amino acid sequence tag to the heterologous amino acid sequence may occur, e.g., by constructing a nucleic acid molecule that comprises: (a) a nucleic acid sequence that encodes the amino acid sequence tag, and (b) a nucleic acid sequence that encodes a heterologous amino acid sequence. Exemplary amino acid sequence tags include, e.g., amino acid sequences that are capable of being post-translationally modified. Other Exemplary amino acid sequence tags include, e.g., amino acid sequences that are capable of being recognized and/or bound by an antibody (or fragment thereof) or other specific binding reagent.

As used herein, the expression “amino acid sequence that is capable of being post-translationally modified” is intended to mean any amino acid sequence, or portion thereof, that can be recognized, in vivo or in vitro, by an enzyme or other molecule that is capable of covalently attaching a chemical entity to one or more amino acids within the amino acid sequence.

As used herein, the term “post-translationally modified protein” is intended to mean at least one protein or polypeptide that has undergone or has been subjected to a post-translational modification. The term “post-translational modification” is intended to mean a modification that can take place in vivo (within a cell) or in vitro (outside a cell) whereby one or more chemical entities are covalently attached to at least one amino acid within the post-translational modification site by means of one or more enzymatic reactions. The site or sites include not only the amino acid that is modified, but any other amino acids, in the proper sequence, that are necessary to allow the post-translational modification to occur.

In the context of the present invention, the amino acid sequences that are capable of being post-translationally modified include amino acid sequences that are capable of being modified by any type of post-translational modification that provides a marker for a protein or polypeptide. The post-translational modifications that are included within the present invention include those that can be used, directly or indirectly, to identify a protein or polypeptide or to isolate it from a mixture of other materials, including other proteins, such as those found in a cell extract or in medium in which a host cell has been cultured and which contains the protein or polypeptide.

Amino acid sequences that are capable of being post-translationally modified include amino acid sequences that can subjected to multiple (e.g., 2, 3, 4, or 5 or more) post-translational modifications.

Preferred post-translational modifications are those that are utilized by a host cell to modify only a small number of proteins. Exemplary post-translational modifications that can be used with the present invention include biotinylation, attachment of 4-phosphopanthetheine, attachment of lipoic acid and attachment of flavins and glycosylation. Further details regarding post-translational modifications of amino acid sequences can be found in U.S. Pat. No. 5,252,466 and the references cited therein.

In a preferred embodiment of the invention, the amino acid sequence that is capable of being post-translationally modified is an amino acid sequence that is capable of being biotinylated (Parrott, M. B. and Barry, M. A., Biochem. Biophys. Res. Comm. 282:993-1000 (2001); Parrott, M. B. and Barry, M. A., Mol. Ther. 1:96-104 (2000)). Amino acid sequences that are capable of being biotinylated are known in the art. Exemplary amino acid sequences that are capable of being biotinylated include, e.g., all or a portion of the Klebsiella pneumoniae oxalacetate decarboxylase α subunit, all or a portion of the Propionibacterium shermanii transcarboxylase 1.3S subunit, and all or a portion of the Escherichia coli biotin carboxyl carrier protein component of acetyl-CoA carboxylase.

According to certain embodiments of the invention, the amino acid sequence that is capable of being biotinylated is an amino acid sequence derived from the C-terminus of the Klebsiella pneumoniae oxalacetate decarboxylase α subunit. In particular embodiments, the amino acid sequence that is capable of being biotinylated is a 72 amino acid peptide derived from the C-terminus of the Klebsiella pneumoniae oxalacetate decarboxylase α subunit (Schwarz, E. et al., J. Biol. Chem. 263:9640-9645 (1988)). This 72 amino acid sequence is also known as “the BIOTAG™.” Biotin is covalently attached to the oxalacetate decarboxylase α subunit and peptide sequencing has identified a single biotin binding site at lysine 561 of the protein. (Schwarz, E. et al., J. Biol. Chem. 263:9640-9645 (1988)). When fused to a heterologous protein, the BIOTAG™ enables the in vivo biotinylation of the recombinant protein of interest. It is preferred that the entire 72 amino acid domain be used to ensure recognition by the cellular biotinylation enzymes. Additional details regarding cellular biotinylation enzymes and the mechanisms of biotinylation can be found in Chapman-Smith, A. and Cronan, J., J. Nutr. 129:477S-484S (1999).

Exemplary amino acid sequences that are capable of being biotinylated are listed in Table I. The nucleotide sequences encoding the exemplary amino acid sequence tags are listed in Table II.

TABLE I
Exemplary Amino Acid Sequences
That are Capable of Being Biotinylated

| Amino Acid | |
|---|---|
| Sequence Tag | Amino Acid Sequence |
| K. pneumoniae | GAGTPVTAPLAGTIWKVLASEGQTVAAGE |
| oxalacetate | VLLILEAMKMETEIRAAQAGTVRGIAVKAG |
| decarboxylase α | DAVAVGDTLMTLA (SEQ ID NO:6) |
| subunit | |
| (Biotag ™) | |
| Mouse pyruvate | KALAVSDLNRAGQRQVFFELNGQLRSILVK |
| decarboxylase | DTQAMKEMHFHPKALKDVKGQIGAPMPGK |
| domain | VIDIKVAAGDKVAKGQPLCVLSAMKMETV |
| | VTSPMEGTIRKVHVTKDMTLEGDDLIL |
| | (SEQ ID NO:7) |
| P. shermanii | MKLKVTVNGTAYDVDVDVDKSHENPMGTI |
| transcarboxylase | LFGGGTGGAPAPRAAGGAGAGKAGEGEIP |
| domain | APLAGTVSKILVKEGDTVKAGQTVLVLEA |
| | MKMETEINAPTDGKVEKVLVKERDAVQGG |
| | QGLIKIG (SEQ ID NO:8) |
| Human acetyl CoA | GSCVEVDVHRLSDGGLLLSYDGSSYTTYM |
| Carboxylase | KEEVDRYRITIGNKTCVFEKENDPSVMRSPS |
| domain | AGKLIQYIVEDGGHVFAGQCYAEIEVMKM |
| | VMTLTAVESGCIHYVKRPGAALDPGCVLA |
| | KMQL (SEQ ID NO:9) |
| E. coli acetyl | MDIRKIKKLIELVEESGISELEISEGEESVRIS |
| CoA carboxylase | RAAPAASFPVMQQAYAAPMMQQPAQSNA |
| BCCP subunit | AAPATVPSMEAPAAAEISGHIVRSPMVGTF |
| | YRTPSPDAKAFIEVGQKVNVGDTLCIVEAM |
| | KMMNQIEADKSGTVKAILVESGQPVEFDEP |
| | LVVIE (SEQ ID NO:10) |

TABLE II
Nucleotide Sequences of Exemplary Amino Acid Sequence Tags

| | Nucleotide Sequence Encoding the |
|---|---|
| Amino Acid Sequence Tag | Amino Acid Sequence Tag |
| K. pneumoniae oxalacetate | ggcgccggcaccccggtgaccgccccgctggcgggcactatctgg |
| decarboxylase α subunit | aaggtgctggccagcgaaggccagacggtggccgcaggcgaggt |
| (Biotag ™) | gctgctgattctggaagccatgaagatggaaaccgaaatccgcgcc |
| | gcgcaggccgggaccgtgcgcggtatcgcggtgaaagccggcga |
| | cgcggtggcggtcggcgacaccctgatgaccctggcg (SEQ ID NO:11) |
| Mouse pyruvate | aaagccctggctgtaagcgacctgaaccgtgctggccagaggcag |
| decarboxylase domain | gtgttctttgaactcaatgggcagcttcgatccattctggttaaagaca |
| | cccaggccatgaaggagatgcacttccatcccaaggctttgaaggat |
| | gtgaagggccaaattggggccccgatgcctgggaaggtcatagac |
| | atcaaggtggcagcaggggacaaggtggctaagggccagcccctc |
| | tgtgtgctcagcgccatgaagatggagactgtggtgacttcgcccat |
| | ggagggcactatccgaaaggttcatgttaccaaggacatgactctgg |
| | aaggcgacgacctcatccta (SEQ ID NO:12) |
| P. shermanii transcarboxylase | atgaaactgaaggtaacagtcaacggcactgcgtatgacgttgacgt |
| domain | tgacgtcgacaagtcacacgaaaacccgatgggcaccatcctgttc |
| | ggcggcggcaccggcggcgcgccggcaccgcgcgcagcaggtg |
| | gcgcaggcgccggtaaggccggagagggcgagattcccgctccg |
| | ctggccggcaccgtctccaagatcctcgtgaaggagggtgacacg |
| | gtcaaggctggtcagaccgtgctcgttctcgaggccatgaagatgga |
| | gaccgagatcaacgctcccaccgacggcaaggtcgagaaggtcct |
| | tgtcaaggagcgtgacgccgtgcagggcggtcagggtctcatcaag |
| | atcggc (SEQ ID NO:13) |
| Human acetyl CoA | ggctcatgtgtagaagtagatgtacatcggctgagtgacggtggact |
| Carboxylase domain | gctcttgtcctatgatggcagcagttacaccacgtatatgaaggagga |
| | agtagacagatatcgcatcacaattggcaataaaacctgtgtgtttga |
| | gaaggaaaatgacccatcggtgatgcgctcaccttctgctgggaagt |
| | taatccagtacattgtagaagatggaggtcatgtgtttgccggccagt |
| | gctatgcagagattgaggtaatgaagatggtaatgactttgacagctg |
| | tggagtctggctgtatccattacgtcaagcgtcctggagcagctcttg |
| | accctggctgtgtactcgccaaaatgcaactg (SEQ ID NO:14) |
| E. coli acetyl CoA | atggatattcgtaagattaaaaaactgatcgagctggttgaagaatca |
| carboxylase BCCP subunit | ggcatctccgaactggaaatttctgaaggcgaagagtcagtacgcat |
| | tagccgtgcagctcctgccgcaagtttccctgtgatgcaacaagctta |
| | cgctgcaccaatgatgcagcagccagctcaatctaacgcagccgct |
| | ccggcgaccgttccttccatggaagcgccagcagcagcggaaatc |
| | agtggtcacatcgtacgttccccgatggttggtactttctaccgcaccc |
| | caagcccggacgcaaaagcgttcatcgaagtgggtcagaaagtca |
| | acgtgggcgataccctgtgcatcgttgaagccatgaaaatgatgaac |
| | cagatcgaagcggacaaatccggtaccgtgaaagcaattctggtcg |
| | aaagtggacaaccggtagaatttgacgagccgctggtcgtcatcgag |
| | (SEQ ID NO:15) |

An amino acid sequence tag, as used herein, may alternatively or additionally be an amino acid sequence that is capable of being recognized by an antibody (or fragment thereof) or other specific binding reagent. The expression “amino acid sequence that is capable of being recognized by an antibody (or fragment thereof) or other specific binding reagent” is intended to mean any amino acid sequence, or portion thereof, to which a particular compound or reagent can interact with or bind to, either covalently or non-covalently. Such amino acid sequences are known in the art. Preferred amino acid sequences that are capable of being recognized by an antibody (or fragment thereof) or other specific binding reagent include, e.g., those that are known in the art as “epitope tags.” An epitope tag may be a natural or an artificial epitope tag. Natural and artificial epitope tags are known in the art, including, e.g., artificial epitopes such as FLAG, Strep, or poly-histidine peptides. FLAG peptides include the sequence Asp-Tyr-Lys-Asp-Asp-Asp-Asp-Lys (SEQ ID NO:16) or Asp-Tyr-Lys-Asp-Glu-Asp-Asp-Lys (SEQ ID NO:17) (Einhauer, A. and Jungbauer, A., J. Biochem. Biophys. Methods 49:1-3:455-465 (2001)). The Strep epitope has the sequence Ala-Trp-Arg-His-Pro-Gln-Phe-Gly-Gly (SEQ ID NO:18). The VSV-G epitope can also be used and has the sequence Tyr-Thr-Asp-Ile-Glu-Met-Asn-Arg-Leu-Gly-Lys (SEQ ID NO:19). Another artificial epitope is a poly-His sequence having six histidine residues (His-His-His-His-His-His (SEQ ID NO:20). Naturally-occurring epitopes include the influenza virus hemagglutinin (HA) sequence Tyr-Pro-Tyr-Asp-Val-Pro-Asp-Tyr-Ala-Ile-Glu-Gly-Arg (SEQ ID NO:21) recognized by the monoclonal antibody 12CA5 (Murray et al., Anal. Biochem. 229:170-179 (1995)) and the eleven amino acid sequence from human c-myc (Myc) recognized by the monoclonal antibody 9E10 (Glu-Gln-Lys-Leu-Leu-Ser-Glu-Glu-Asp-Leu-Asn (SEQ ID NO:22) (Manstein et al., Gene 162:129-134 (1995)). Another useful epitope is the tripeptide Glu-Glu-Phe (SEQ ID NO:23) which is recognized by the monoclonal antibody YL 1/2. (Stammers et al. FEBS Lett. 283:298-302(1991)).

The nucleic acid molecules of the invention may include a variety of elements. The nucleic acid molecule of the invention preferably comprises one or more nucleic acid sequences which encode one or more amino acid sequence tags. The nucleic acid molecules may also comprise one or more recombination sites and/or one or more topoisomerase recognition sites and/or one or more topoisomerases.

The nucleic acid molecules of the invention may also comprise one or more selectable markers, one or more cloning sites, one or more restriction sites, one or more promoters, one or more operators (e.g., a tet operator, a galactose operon operator, a lac operon operator, and the like), one or more operons, one or more origins of replication, one or more nucleotide sequences that encode a gene product which allows for negative selection, one or more nucleotide sequences which encode a repressor of at least one promoter, and one or more genes or gene products. Additional elements useful for molecular biology applications will be known to those skilled in the art and can be included within the nucleic acid molecules of the invention as well. The exact combination of elements, and their relative locations within the nucleic acid molecules of the invention, may vary depending on the intended uses of the nucleic acid molecules.

As used herein, a selectable marker is intended to include a nucleic acid segment that allows one to select for or against a molecule (e.g., a replicon) or a cell that contains it, often under particular conditions. These markers can encode an activity, such as, but not limited to, production of RNA, peptide, or protein, or can provide a binding site for RNA, peptides, proteins, inorganic and organic compounds or compositions and the like. Examples of selectable markers include but are not limited to: (1) nucleic acid segments that encode products which provide resistance against otherwise toxic compounds (e.g., antibiotics); (2) nucleic acid segments that encode products which are otherwise lacking in the recipient cell (e.g., tRNA genes, auxotrophic markers); (3) nucleic acid segments that encode products which suppress the activity of a gene product; (4) nucleic acid segments that encode products which can be readily identified (e.g., phenotypic markers such as (-galactosidase, green fluorescent protein (GFP), enhanced green fluorescent protein (EGFP), and cell surface proteins); (5) nucleic acid segments that bind products which are otherwise detrimental to cell survival and/or function; (6) nucleic acid segments that otherwise inhibit the activity of any of the nucleic acid segments described in Nos. 1-5 above (e.g., antisense oligonucleotides); (7) nucleic acid segments that bind products that modify a substrate (e.g. restriction endonucleases); (8) nucleic acid segments that can be used to isolate or identify a desired molecule (e.g. specific protein binding sites); (9) nucleic acid segments that encode a specific nucleotide sequence which can be otherwise non-functional (e.g., for PCR amplification of subpopulations of molecules); (10) nucleic acid segments, which when absent, directly or indirectly confer resistance or sensitivity to particular compounds; and/or (11) nucleic acid segments that encode products which are toxic in recipient cells.

Exemplary selectable markers that can be included within the nucleic acid molecules of the invention include, e.g., a gene encoding a product that confers resistance to chloramphenicol, e.g., a chloramphenicol resistance gene (CmR), a gene encoding a product that confers resistance to ampicillin, e.g., a gene which encodes β-lactamase, a gene encoding a product that confers resistance to other antibiotic compounds, a ccdB gene or other toxic genes (allowing for counterselection of the nucleic acid molecule), and a gene encoding a product that confers resistance to blasticidin, e.g., a bsd resistance gene. Any other selectable marker gene known in the art can be include within the nucleic acid molecules of the invention.

A “cloning site,” as used herein includes any nucleic acid regions which contain at least one restriction endonuclease cleavage sites. The nucleic acid molecules of the invention may also comprise “multiple cloning sites.” A multiple cloning site is any nucleic acid region which contains two or more restriction endonuclease cleavage sites. “Restriction endonuclease cleavage sites are also referred to in the art as “restriction sites.”

As used herein, a promoter is an example of a transcriptional regulatory sequence, and is specifically a nucleic acid sequence generally described as the 5′-region of a gene located proximal to the start codon. The transcription of an adjacent nucleic acid segment is initiated at the promoter region. A repressible promoter's rate of transcription decreases in response to a repressing agent. An inducible promoter's rate of transcription increases in response to an inducing agent. A constitutive promoter's rate of transcription is not specifically regulated, though it can vary under the influence of general metabolic conditions.

Any promoter known to those skilled in the art can be included in the nucleic acid molecules of the invention. Exemplary promoters include, e.g., the T7 promoter, the human cytomegalovirus (CMV) immediate early enhancer/promoter, the SV40 early promoter, a metallothionein (MT) promoter, including, e.g., the Drosophila MT promoter. Other exemplary promoters include those that are inducible by, or can be repressed by, e.g., certain carbon sources (e.g., glucose, galactose, arabinose, etc.), salts, temperature changes (e.g., temperatures greater than or less than the normal physiological growth temperature), and other molecules.

A number of operators are known in the art and can be included in the nucleic acid molecules of the invention. An example of an operator suitable for use with the invention is the tryptophan operator of the tryptophan operon of E. coli. The tryptophan repressor, when bound to two molecules of tryptophan, binds to the E. coli tryptophan operator and, when suitably positioned with respect to the promoter, blocks transcription. Another example of an operator suitable for use with the invention is operator of the E. coli tetracycline operon. Components of the tetracycline resistance system of E. coli have also been found to function in eukaryotic cells and have been used to regulate gene expression. For example, the tetracycline repressor, which binds to tetracycline operator in the absence of tetracycline and represses gene transcription, has been expressed in plant cells at sufficiently high concentrations to repress transcription from a promoter containing tetracycline operator sequences (Gatz et al., Plants 2:397-404 (1992)). The tetracycline regulated expression systems are described, for example in U.S. Pat. No. 5,789,156, the entire disclosure of which is incorporated herein by reference. Additional examples of operators which can be used with the invention include the Lac operator and the operator of the molybdate transport operator/promoter system of E. coli (see, e.g., Cronin et al., Genes Dev. 15:1461-1467 (2001) and Grunden et al., J. Biol. Chem., 274:24308-24315 (1999)).

Thus, in particular embodiments, the invention provides nucleic acid molecules that contain one or more operators which can be used to regulate expression in prokaryotic or eukaryotic cells. As one skilled in the art would recognize, when a nucleic acid molecule which contains an operator is placed under conditions in which transcriptional machinery is present, either in vivo or in vitro, regulation of expression will often be modulated by contacting the nucleic acid molecule with a repressor and one or more metabolites which facilitate binding of an appropriate repressor to the operator. Thus, the invention further provides nucleic acid molecules which encode repressors which modulate the function of operators.

The nucleic acid molecules of the invention may comprise one or more genes or partial genes. As used herein, a gene is a nucleic acid sequence that contains information necessary for expression of a polypeptide, protein or functional RNA (e.g., a ribozyme, tRNA, rRNA, mRNA, etc.). It includes the promoter and the structural gene open reading frame sequence (orf) as well as other sequences involved in expression of the protein. As used herein, a structural gene refers to a nucleic acid sequence that is transcribed into messenger RNA that is then translated into a sequence of amino acids characteristic of a specific polypeptide.

The range of positions of the various elements of the nucleic acid molecules of the invention, relative to one another, will be appreciated by persons having ordinary skill in the art. For example, a nucleic acid molecule within the scope of the invention may comprise (a) one or more recombination sites; and (b) one or more nucleic acid sequences which encode one or more amino acid sequence tags. In a preferred embodiment, elements (a) and (b) will be positioned relative to one another such that a nucleic acid sequence of interest can be inserted at or within 20 nucleotides of said one or more recombination sites, thereby producing a polynucleotide construct that encodes a fusion protein. Such fusion protein may comprise: (i) the amino acid sequence tag, and (ii) the amino acid sequence encoded by said nucleic acid sequence of interest.

Similarly, a nucleic acid molecule within the scope of the invention may comprise (a) one or more topoisomerase recognition sites and/or one or more topoisomerases; and (b) one or more nucleic acid sequences which encode one or more amino acid sequence tags. In a preferred embodiment, elements (a) and (b) will be positioned relative to one another such that a nucleic acid sequence of interest can be inserted at or within 20 nucleotides of said one or more topoisomerase recognition sites and/or at or within 20 nucleotides of the position of said one or more topoisomerases, thereby producing a polynucleotide construct that encodes a fusion protein. Such fusion protein may comprise: (i) the amino acid sequence tag, and (ii) the amino acid sequence encoded by said nucleic acid sequence of interest.

Similarly, a nucleic acid molecule within the scope of the invention may comprise (a) one or more recombination sites; (b) one or more topoisomerase recognition sites and/or one or more topoisomerases; and (c) one or more nucleic acid sequences which encode one or more amino acid sequence tags. In a preferred embodiment, elements (a), (b) and (c) will be positioned relative to one another such that a nucleic acid sequence of interest can be inserted at or within 20 nucleotides of said one or more recombination sites, thereby producing a polynucleotide construct that encodes a fusion protein. Such fusion protein may comprise: (i) the amino acid sequence tag, and (ii) the amino acid sequence encoded by said nucleic acid sequence of interest. In another preferred embodiment, elements (a), (b) and (c) will be positioned relative to one another such that a nucleic acid sequence of interest can be inserted at or within 20 nucleotides of said one or more topoisomerase recognition sites and/or at or within 20 nucleotides of the position of said one or more topoisomerases, thereby producing a polynucleotide construct that encodes a fusion protein. Such fusion protein may comprise: (i) the amino acid sequence tag, and (ii) the amino acid sequence encoded by said nucleic acid sequence of interest.

In certain embodiments, the nucleic acid molecules of the invention will comprise a nucleic acid sequence that encodes an amino acid sequence that is capable of being recognized and/or cleaved by one or more proteases. Amino acid sequences that can be recognized and/or cleaved by one or more proteases are known in the art. Exemplary amino acid sequences are those that are recognized by the following proteases: factor VIIa, factor IXa, factor Xa, APC, t-PA, u-PA, trypsin, chymotrypsin, enterokinase, pepsin, cathepsin B,H,L,S,D, cathepsin G, renin, angiotensin converting enzyme, matrix metalloproteases (collagenases, stromelysins, gelatinases), macrophage elastase, Cir, and Cis. The amino acid sequences that are recognized by the aforementioned proteases are known in the art. Exemplary sequences recognized by certain proteases can be found, e.g., in U.S. Pat. No. 5,811,252. A preferred amino acid sequence that is capable of being recognized and/or cleaved by a protease is the enterokinase (EK) recognition site (Asp-Asp-Asp-Asp-Lys (SEQ ID NO:24).

The invention therefore also includes nucleic acid molecules comprising: (a) one or more recombination sites; (b) one or more nucleic acid sequences which encode one or more amino acid sequence tags; and (c) one or more nucleic acid sequences that encodes an amino acid sequence that is capable of being recognized and/or cleaved by one or more proteases.

The invention also includes nucleic acid molecules comprising: (a) one or more topoisomerase recognition sites and/or one or more topoisomerases; (b) one or more nucleic acid sequences which encode one or more amino acid sequence tags; and (c) one or more nucleic acid sequence that encodes an amino acid sequence that is capable of being recognized and/or cleaved by one or more proteases. In a preferred aspect, the nucleic acid sequence that encodes an amino acid sequence that is capable of being recognized and/or cleaved by one or more proteases is positioned such that, upon cleavage, the amino acid sequence tag is completely or partially removed from the amino acid sequence of interest. In another aspect, the nucleic acid sequence that encodes an amino acid sequence that is capable of being recognized and/or cleaved by one or more proteases is positioned such that, upon cleavage, other sequences (e.g., topoisomerase recognition sequences and/or recombination sites) may be removed from the amino acid sequence of interest.

The invention also includes nucleic acid molecules comprising: (a) one or more recombination sites; (b) one or more topoisomerase recognition sites and/or one or more topoisomerases; (c) one or more nucleic acid sequences which encode one or more amino acid sequence tags; and (d) one or more nucleic acid sequence that encodes an amino acid sequence that is capable of being recognized and/or cleaved by one or more proteases. In a preferred aspect, the nucleic acid sequence that encodes an amino acid sequence that is capable of being recognized and/or cleaved by one or more proteases is positioned such that, upon cleavage, the amino acid sequence tag is completely or partially removed from the amino acid sequence of interest. In another aspect, the nucleic acid sequence that encodes an amino acid sequence that is capable of being recognized and/or cleaved by one or more proteases is positioned such that, upon cleavage, other sequences (e.g., topoisomerase recognition sequences and/or recombination sites) may be removed from the amino acid sequence of interest.

The position of a nucleic acid sequence that encodes an amino acid sequence that is capable of being recognized and/or cleaved by one or more proteases, relative to the other elements of the nucleic acid molecules of the invention will be such that a nucleic acid sequence of interest can be inserted at or within 20 nucleotides of said one or more recombination sites, or at or within 20 nucleotides of said one or more topoisomerase recognition sites and/or at or within 20 nucleotides of the position of said one or more topoisomerases, thereby producing a polynucleotide construct that encodes a fusion protein. Such fusion protein may comprise: (i) said amino acid sequence that is capable of being cleaved by one or more proteases, flanked on one side by (ii) said amino acid sequence tag, and on the other side by (iii) the amino acid sequence encoded by said nucleic acid sequence of interest.

This arrangement of elements will enable the production of a fusion protein of interest comprising an amino acid sequence tag, and will also enable the subsequent cleavage of the fusion protein by a protease, thereby separating the amino acid sequence tag from the amino acid sequence encoded by said nucleic acid sequence of interest. If the fusion protein is a fusion protein that is capable of being post-translationally modified, cleavage by the protease can be accomplished either before or after the post-translational modification of the fusion protein.

In addition to comprising one or more nucleic acid sequences which encode one or more amino acid sequence tags and/or one or more recombination sites and/or one or more topoisomerase recognition sites and/or one or more topoisomerases and/or one or more nucleic acid sequence that encodes an amino acid sequence that is capable of being cleaved by one or more proteases, the nucleic acid molecules of the invention may further comprise additional elements. Exemplary additional elements that can be included within the nucleic acid molecules of the invention include, e.g., one or more promoters, one or more selectable markers, one or more origins of replication, one or more operators, one or more enhancers, one or more ribosome binding sites, one or more initiation codons, one or more nucleic acid sequences of interest (e.g., one or more nucleic acid sequences encoding one or more protein or polypeptides of interest), one or more polyadenylation signals, and/or one or more transcription termination regions. As understood by those skilled in the art, other elements may be included within the nucleic acid molecules of the invention depending on the circumstances under which the nucleic acids are intended to be used.

The possible arrangements of the various elements of the nucleic acid molecules of the invention, relative to one another, will be appreciated by persons having ordinary skill in the art. Non-limiting, exemplary arrangements are as follows:

Exemplary arrangement I: (a) one or more promoters—(b) one or more nucleic acid sequences which encode one or more amino acid sequence tags—(c) one or more nucleic acid sequences that encodes an amino acid sequence that is capable of being cleaved by one or more proteases—(d) one or more recombination sites and/or one or more topoisomerase recognition sites and/or one or more topoisomerases—(e) one or more polyadenylation signals and/or one or more transcription termination regions.

Exemplary arrangement II: (a) one or more promoters—(b) one or more nucleic acid sequences which encode one or more amino acid sequence tags—(c) one or more nucleic acid sequences that encodes an amino acid sequence that is capable of being cleaved by one or more proteases—(d) one or more recombination sites and/or one or more topoisomerase recognition sites and/or one or more topoisomerases—(e) one or more nucleic acid sequences of interest—(f) one or more polyadenylation signals and/or one or more transcription termination regions.

Exemplary arrangement III: (a) one or more promoters—(b) one or more nucleic acid sequences which encode one or more amino acid sequence tags—(c) one or more recombination sites and/or one or more topoisomerase recognition sites and/or one or more topoisomerases—(d) one or more polyadenylation signals and/or one or more transcription termination regions.

Exemplary arrangement IV: (a) one or more promoters—(b) one or more nucleic acid sequences which encode one or more amino acid sequence tags—(c) one or more recombination sites and/or one or more topoisomerase recognition sites and/or one or more topoisomerases—(d) one or more nucleic acid sequences of interest—(e) one or more polyadenylation signals and/or one or more transcription termination regions.

Exemplary arrangement V: (a) one or more promoters—(b) one or more recombination sites and/or one or more topoisomerase recognition sites and/or one or more topoisomerases—(c) one or more nucleic acid sequences that encodes an amino acid sequence that is capable of being cleaved by one or more proteases—(d) one or more nucleic acid sequences which encode one or more amino acid sequence tags—(e) one or more polyadenylation signals and/or one or more transcription termination regions.

Exemplary arrangement VI: (a) one or more promoters—(b) one or more nucleic acid sequences of interest—(c) one or more recombination sites and/or one or more topoisomerase recognition sites and/or one or more topoisomerases—(d) one or more nucleic acid sequences that encodes an amino acid sequence that is capable of being cleaved by one or more proteases—(e) one or more nucleic acid sequences which encode one or more amino acid sequence tags—(f) one or more polyadenylation signals and/or one or more transcription termination regions.

Exemplary arrangement VII: (a) one or more promoter—(b) one or more recombination sites and/or one or more topoisomerase recognition sites and/or one or more topoisomerases—(c) one or more nucleic acid sequences which encode one or more amino acid sequence tags—(d) one or more polyadenylation signals and/or one or more transcription termination regions.

Exemplary arrangement VIII: (a) one or more promoters—(b) one or more nucleic acid sequences of interest—(c) one or more recombination sites and/or one or more topoisomerase recognition sites and/or one or more topoisomerases—(d) one or more nucleic acid sequences which encode one or more amino acid sequence tags—(e) one or more polyadenylation signals and/or one or more transcription termination regions.

In the foregoing exemplary arrangements, it will be understood by those skilled in the art that one or more additional elements may be included between any of the specifically listed elements, and/or that any of the specifically listed elements may be omitted. It will also be understood that many variations on these exemplary arrangements are possible (e.g., addition and/or omission of various elements) such that the nucleic acid molecules of the invention will allow the insertion of a nucleic acid sequence of interest and/or the production of a polynucleotide construct that encodes a desired fusion protein.

Persons of ordinary skill in the art will readily understand how close together, or how far apart, the elements of the nucleic acid molecules of the invention can be in order to permit the insertion of a nucleic acid sequence of interest and/or the production of a polynucleotide construct that encodes a desired fusion protein. For example, any two or more of the foregoing elements may be arranged within the nucleic acid molecules of the invention such that they are within about 500 nucleotides of one another. In certain embodiments, any two or more elements of the nucleic acid molecules will be within about 400 nucleotides of one another, within about 300 nucleotides of one another, within about 200 nucleotides of one another, within about 100 nucleotides of one another, within about 50 nucleotides of one another, within about 40 nucleotides of one another, within about 30 nucleotides of one another, within about 20 nucleotides of one another, within about 10 nucleotides of one another, within about 5 nucleotides of one another, within about 4 nucleotides of one another, within about 3 nucleotides of one another, within about 2 nucleotides of one another, or within about 1 nucleotide of one another. The elements of the nucleic acid molecules of the invention may alternatively be directly adjacent to one another (e.g., with no nucleotides separating them), as long as such an arrangement permits the insertion of a nucleic acid sequence of interest and/or the production of a polynucleotide construct that encodes a desired fusion protein.

It will also be appreciated that the nucleic acid sequence of interest will be preferably designed such that, when it is inserted at or within 20 nucleotides of said one or more recombination sites or at or within 20 nucleotides of said one or more topoisomerase recognition sites and/or at or within 20 nucleotides of the position of said one or more topoisomerases, the nucleic acid sequence of interest is in frame with the nucleic acid sequence tag.

The nucleic acid molecules of the invention are useful, e.g., in the production of fusion proteins that comprise one or more amino acid sequence tags. The fusion protein may be, e.g., an N-terminal fusion protein (e.g., wherein an amino acid sequence tag is covalently attached at or near the N-terminus of the amino acid sequence encoded by said nucleic acid sequence of interest). The fusion protein may also be, e.g., a C-terminal fusion protein (e.g., wherein an amino acid sequence tag is covalently attached at or near the C-terminus of the amino acid sequence encoded by said nucleic acid sequence of interest). The fusion protein may also be, e.g., an N-terminal and C-terminal fusion protein (e.g., wherein an amino acid sequence tag is covalently attached at or near the N-terminus of the amino acid sequence encoded by said nucleic acid sequence of interest and an amino acid sequence tag is covalently attached at or near the C-terminus of the amino acid sequence encoded by said nucleic acid sequence of interest).

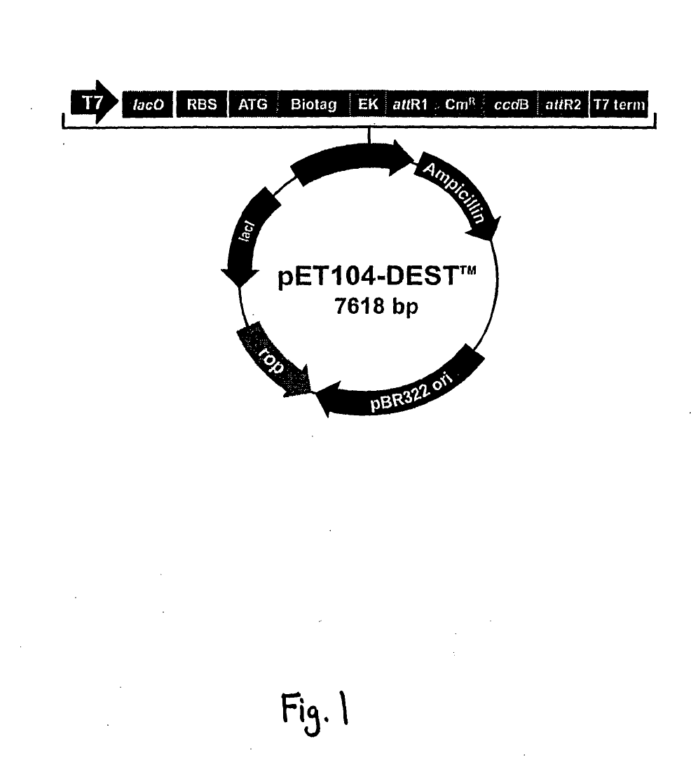

The nucleic acid molecules of the invention may comprise one or more (e.g., 2, 3, 4, 5, 6, 7, 8, etc.) recombination sites. As used herein, a recombination site is a recognition sequence on a nucleic acid molecule participating in an integration/recombination reaction by recombination proteins. Recombination sites are discrete sections or segments of nucleic acid on the participating nucleic acid molecules that are recognized and bound by a site-specific recombination protein during the initial stages of integration or recombination. For example, the recombination site for Cre recombinase is loxp which is a 34 base pair sequence comprised of two 13 base pair inverted repeats (serving as the recombinase binding sites) flanking an 8 base pair core sequence. See FIG. 1 of Sauer, B., Curr. Opin. Biotech. 5:521-527 (1994). Other examples of recognition sequences include the attB, attP, attL, and attR sequences described herein, and mutants, fragments, variants and derivatives thereof, which are recognized by the recombination protein (Int and by the auxiliary proteins integration host factor (IHF), FIS and excisionase (Xis). See Landy, Curr. Opin. Biotech. 3:699-707 (1993).

Recombination sites for use in the invention may be any nucleic acid sequence that can serve as a substrate in a recombination reaction. Such recombination sites may be wild-type or naturally occurring recombination sites or modified or mutant recombination sites. Examples of recombination sites for use in the invention include, but are not limited to, phage-lambda recombination sites (such as attP, attB, attL, and attR and mutants or derivatives thereof) and recombination sites from other bacteriophage such as phi80, P22, P2, 186, P4 and P1 (including lox sites such as loxP and loxP511). Novel mutated att sites (e. g., attB 1-10, attP 1-10, attR 1-10 and attL 1-10) are described in International Patent Application PCT/US00/05432, which is specifically incorporated herein by reference. Other recombination sites having unique specificity (i.e., a first site will recombine with its corresponding site and will not recombine with a second site having a different specificity) are known to those skilled in the art and may be used to practice the present invention.

Corresponding recombination proteins for these systems may be used in accordance with the invention with the indicated recombination sites. Other systems providing recombination sites and recombination proteins for use in the invention include the FLP/FRT system from Saccharomyces cerevisiae, the resolvase family (e.g., (, Tn3 resolvase, Hin, Gin and Cin), and IS231 and other Bacillus thuringiensis transposable elements. Other suitable recombination systems for use in the present invention include the XerC and XerD recombinases and the psi, dif and cer recombination sites in E. coli. Other suitable recombination sites may be found in U.S. Pat. Nos. 5,851,808 and 6,410,317 which are specifically incorporated herein by reference. Preferred recombination proteins and mutant or modified recombination sites for use in the invention include those described in U.S. Pat. Nos. 5,888,732, 6,171,861, 6,143,557, 6,270,969 and 6,277,608, and commonly owned, co-pending U.S. application Ser. No. 09/438,358 (filed Nov. 12, 1999), Ser. No. 09/517,466 (filed Mar. 2, 2000), Ser. No. 09/695,065 (filed Oct. 25, 2000), Ser. No. 09/732,914 (filed Dec. 11, 2000), and international application Nos. WO 01/11058 and WO 01/42509, the disclosures of all of which are incorporated herein by reference in their entireties, as well as those associated with the GATEWAY™ Cloning Technology and Echo™ Cloning Technology available from Invitrogen Corporation (Carlsbad, Calif.).

The nucleic acid molecules of the invention may comprise one or more (e.g., 2, 3, 4, 5, 6, 7, 8, etc.) topoisomerase recognition sites and/or one or more topoisomerases. As used herein, a topoisomerase recognition sequence (alternatively and equivalently referred to herein as a “topoisomerase recognition site”) is a particular sequence to which a topoisomerase recognizes and binds. Examples of topoisomerase recognition sites include, but are not limited to, the sequence 5′-GCAACTT-3′ that is recognized by E. coli topoisomerase III (a type I topoisomerase); the sequence 5′-(C/T)CCTT-3′ which is a topoisomerase recognition site that is bound specifically by most poxvirus topoisomerases, including vaccinia virus DNA topoisomerase I; and others that are known in the art as discussed elsewhere herein.

Topoisomerases are categorized as type I, including type IA and type IB topoisomerases, which cleave a single strand of a double stranded nucleic acid molecule, and type II topoisomerases (gyrases), which cleave both strands of a nucleic acid molecule. Type IA and IB topoisomerases cleave one strand of a nucleic acid molecule. Cleavage of a nucleic acid molecule by type IA topoisomerases generates a 5′ phosphate and a 3′ hydroxyl at the cleavage site, with the type IA topoisomerase covalently binding to the 5′ terminus of a cleaved strand. In comparison, cleavage of a nucleic acid molecule by type IB topoisomerases generates a 3′ phosphate and a 5′ hydroxyl at the cleavage site, with the type IB topoisomerase covalently binding to the 3′ terminus of a cleaved strand. As disclosed herein, type I and type II topoisomerases, as well as catalytic domains and mutant forms thereof, are useful for generating ds recombinant nucleic acid molecules covalently linked in both strands according to a method of the invention.

Type IA topoisomerases include E. coli topoisomerase I, E. coli topoisomerase III, eukaryotic topoisomerase II, archeal reverse gyrase, yeast topoisomerase III, Drosophila topoisomerase III, human topoisomerase III, Streptococcus pneumoniae topoisomerase III, and the like, including other type IA topoisomerases (see Berger, Biochim. Biophys. Acta 1400:3-18, 1998; DiGate and Marians, J. Biol. Chem. 264:17924-17930, 1989; Kim and Wang, J. Biol. Chem. 267:17178-17185, 1992; Wilson et al., J. Biol. Chem. 275:1533-1540, 2000; Hanai et al., Proc. Natl. Acad. Sci., USA 93:3653-3657, 1996, U.S. Pat. No. 6,277,620, each of which is incorporated herein by reference). E. coli topoisomerase III, which is a type IA topoisomerase that recognizes, binds to and cleaves the sequence 5′-GCAACTT-3′, can be particularly useful in a method of the invention (Zhang et al., J. Biol. Chem. 270:23700-23705, 1995, which is incorporated herein by reference). A homolog, the traE protein of plasmid RP4, has been described by Li et al., J. Biol. Chem. 272:19582-19587 (1997) and can also be used in the practice of the invention. A DNA-protein adduct is formed with the enzyme covalently binding to the 5′-thymidine residue, with cleavage occurring between the two thymidine residues.

Type IB topoisomerases include the nuclear type I topoisomerases present in all eukaryotic cells and those encoded by vaccinia and other cellular poxviruses (see Cheng et al., Cell 92:841-850, 1998, which is incorporated herein by reference). The eukaryotic type IB topoisomerases are exemplified by those expressed in yeast, Drosophila and mammalian cells, including human cells (see Caron and Wang, Adv. Pharmacol. 29B,:271-297, 1994; Gupta et al., Biochim. Biophys. Acta 1262:1-14, 1995, each of which is incorporated herein by reference; see, also, Berger, supra, 1998). Viral type IB topoisomerases are exemplified by those produced by the vertebrate poxviruses (vaccinia, Shope fibroma virus, ORF virus, fowlpox virus, and molluscum contagiosum virus), and the insect poxvirus (Amsacta moorei entomopoxvirus) (see Shuman, Biochim. Biophys. Acta 1400:321-337, 1998; Petersen et al., Virology 230:197-206, 1997; Shuman and Prescott, Proc. Natl. Acad. Sci., USA 84:7478-7482, 1987; Shuman, J. Biol. Chem. 269:32678-32684, 1994; U.S. Pat. No. 5,766,891; PCT/US95/16099; PCT/US98/12372,, each of which is incorporated herein by reference; see, also, Cheng et al., supra, 1998).

Type II topoisomerases include, for example, bacterial gyrase, bacterial DNA topoisomerase IV, eukaryotic DNA topoisomerase II, and T-even phage encoded DNA topoisomerases (Roca and Wang, Cell 71:833-840, 1992; Wang, J. Biol. Chem. 266:6659-6662, 1991, each of which is incorporated herein by reference; Berger, supra, 1998). Like the type IB topoisomerases, the type II topoisomerases have both cleaving and ligating activities. In addition, like type IB topoisomerase, substrate nucleic acid molecules can be prepared such that the type II topoisomerase can form a covalent linkage to one strand at a cleavage site. For example, calf thymus type II topoisomerase can cleave a substrate nucleic acid molecule containing a 5′ recessed topoisomerase recognition site positioned three nucleotides from the 5′ end, resulting in dissociation of the three nucleotide sequence 5′ to the cleavage site and covalent binding the of the topoisomerase to the 5′ terminus of the nucleic acid molecule (Andersen et al., supra, 1991). Furthermore, upon contacting such a type II topoisomerase charged nucleic acid molecule with a second nucleotide sequence containing a 3′ hydroxyl group, the type II topoisomerase can ligate the sequences together, and then is released from the recombinant nucleic acid molecule. As such, type II topoisomerases also are useful in the nucleic acid molecules and methods of the invention.

Structural analysis of topoisomerases indicates that the members of each particular topoisomerase families, including type IA, type IB and type II topoisomerases, share common structural features with other members of the family (Berger, supra, 1998). In addition, sequence analysis of various type IB topoisomerases indicates that the structures are highly conserved, particularly in the catalytic domain (Shuman, supra, 1998; Cheng et al., supra, 1998; Petersen et al., supra, 1997). For example, a domain comprising amino acids 81 to 314 of the 314 amino acid vaccinia topoisomerase shares substantial homology with other type IB topoisomerases, and the isolated domain has essentially the same activity as the full length topoisomerase, although the isolated domain has a slower turnover rate and lower binding affinity to the recognition site (see Shuman, supra, 1998; Cheng et al., supra, 1998). In addition, a mutant vaccinia topoisomerase, which is mutated in the amino terminal domain (at amino acid residues 70 and 72) displays identical properties as the full length topoisomerase (Cheng et al., supra, 1998). In fact, mutation analysis of vaccinia type IB topoisomerase reveals a large number of amino acid residues that can be mutated without affecting the activity of the topoisomerase, and has identified several amino acids that are required for activity (Shuman, supra, 1998). In view of the high homology shared among the vaccinia topoisomerase catalytic domain and the other type IB topoisomerases, and the detailed mutation analysis of vaccinia topoisomerase, it will be recognized that isolated catalytic domains of the type IB topoisomerases and type IB topoisomerases having various amino acid mutations can be included with the nucleic acid molecules and methods of the invention.

The various topoisomerases exhibit a range of sequence specificity. For example, type II topoisomerases can bind to a variety of sequences, but cleave at a highly specific recognition site (see Andersen et al., J. Biol. Chem. 266:9203-9210, 1991, which is incorporated herein by reference.). In comparison, the type IB topoisomerases include site specific topoisomerases, which bind to and cleave a specific nucleotide sequence (“topoisomerase recognition site”). Upon cleavage of a nucleic acid molecule by a topoisomerase, for example, a type IB topoisomerase, the energy of the phosphodiester bond is conserved via the formation of a phosphotyrosyl linkage between a specific tyrosine residue in the topoisomerase and the 3′ nucleotide of the topoisomerase recognition site. Where the topoisomerase cleavage site is near the 3′ terminus of the nucleic acid molecule, the downstream sequence (3′ to the cleavage site) can dissociate, leaving a nucleic acid molecule having the topoisomerase covalently bound to the newly generated 3′ end.

The nucleic acid molecules of the invention are useful, e.g., for the production of fusion proteins. As used herein, the term “fusion protein” is intended to include any polypeptide which contains amino acids derived from at least two different polypeptides. The nucleic acid molecules of the invention are especially useful, e.g., for producing fusion proteins comprising (i) one or more amino acid sequence tags, and (ii) one or more amino acid sequence encoded by one or more nucleic acid sequences of interest.

The invention also includes vectors comprising any of the nucleic acid molecules described herein. As used herein, a vector is a nucleic acid molecule (preferably DNA) that provides a useful biological or biochemical property to an insert. Examples include plasmids, phages, autonomously replicating sequences (ARS), centromeres, and other sequences which are able to replicate or be replicated in vitro or in a host cell, or to convey a desired nucleic acid segment to a desired location within a host cell. A Vector can have one or more restriction endonuclease recognition sites at which the sequences can be cut in a determinable fashion without loss of an essential biological function of the vector, and into which a nucleic acid fragment can be spliced in order to bring about its replication and cloning. Vectors can further provide primer sites, e.g., for PCR, transcriptional and/or translational initiation and/or regulation sites, recombinational signals, replicons, selectable markers, etc. Clearly, methods of inserting a desired nucleic acid fragment which do not require the use of recombination, transpositions or restriction enzymes (such as, but not limited to, UDG cloning of PCR fragments (U.S. Pat. No. 5,334,575, entirely incorporated herein by reference), TA Cloning® brand PCR cloning (Invitrogen Corporation, Carlsbad, Calif.) (also known as direct ligation cloning), and the like) can also be applied to clone a fragment into a cloning vector to be used according to the present invention. The cloning vector can further contain one or more selectable markers suitable for use in the identification of cells transformed with the cloning vector.

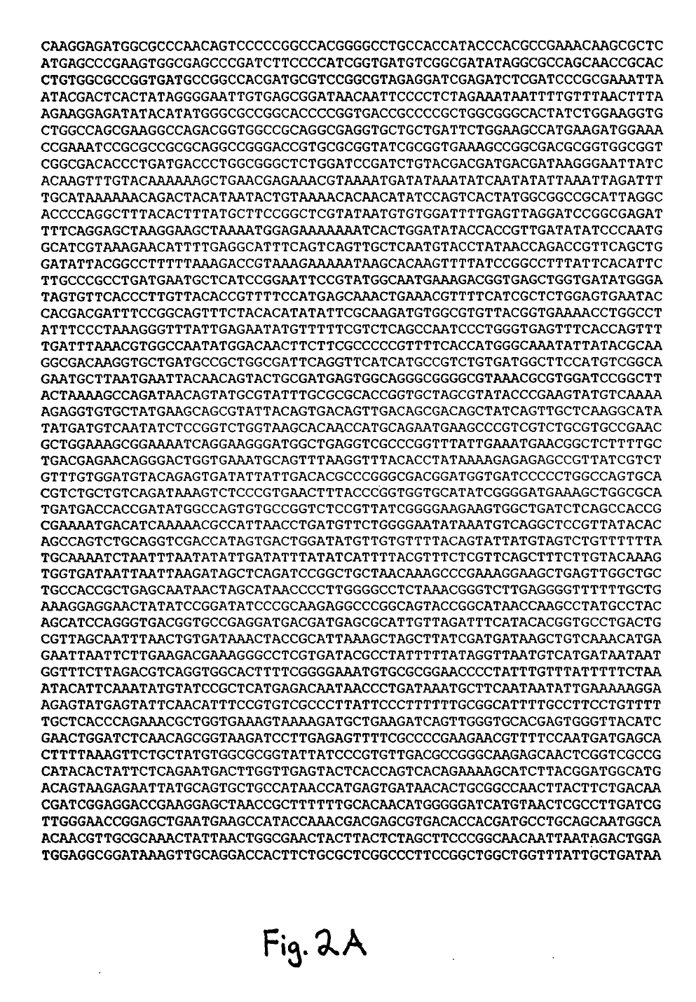

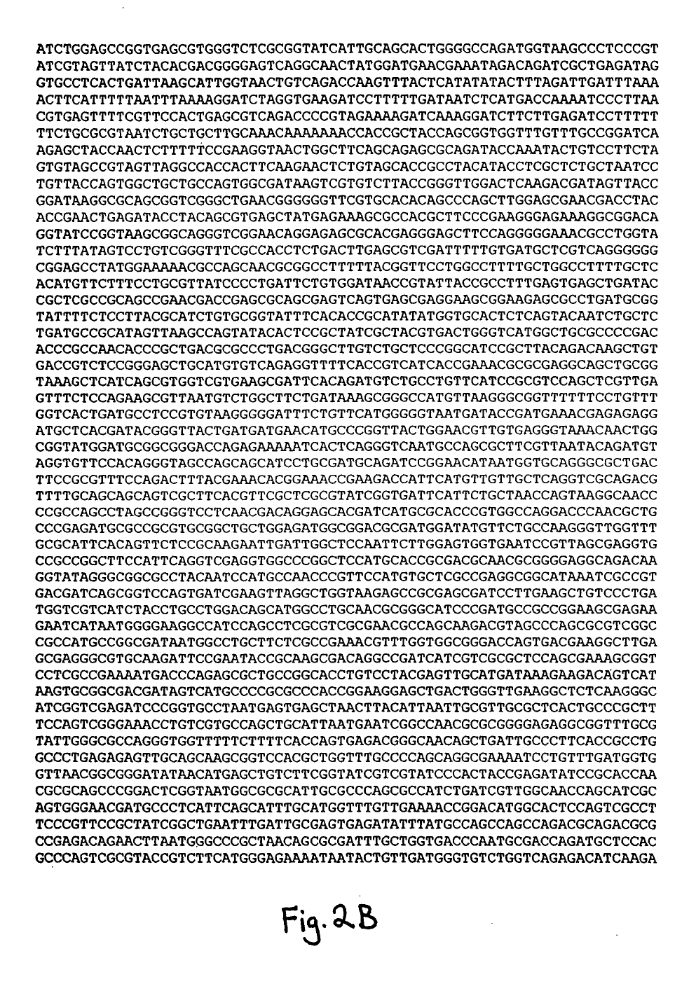

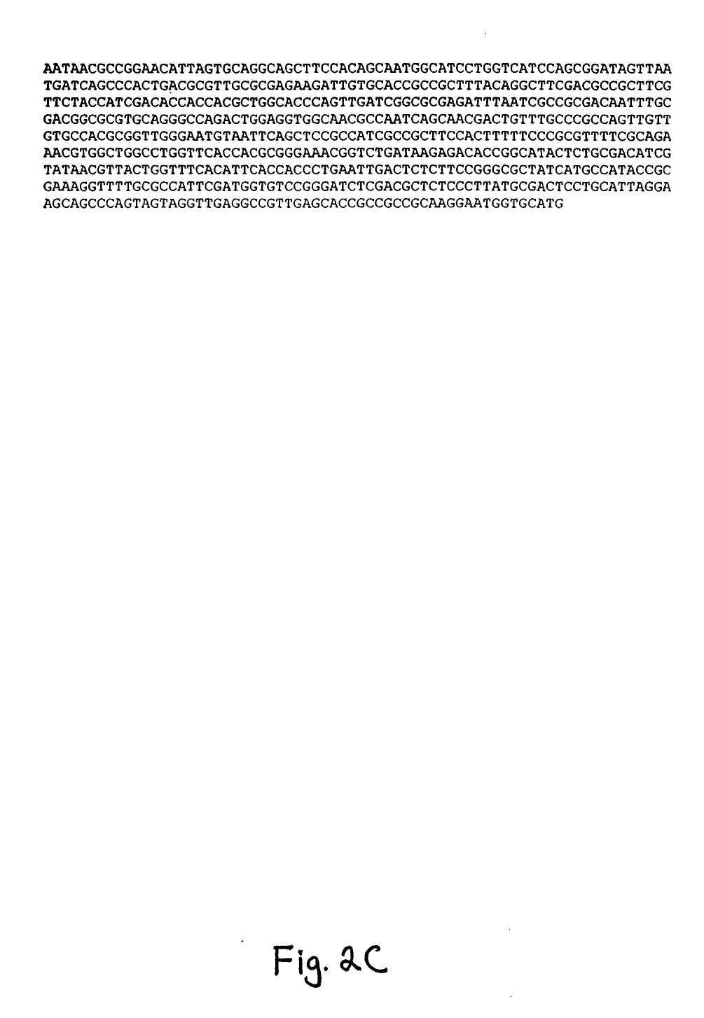

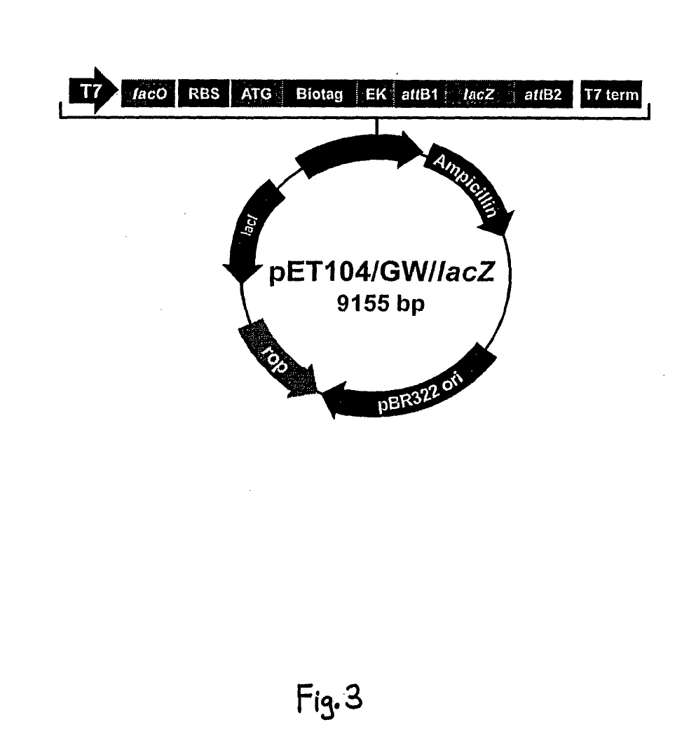

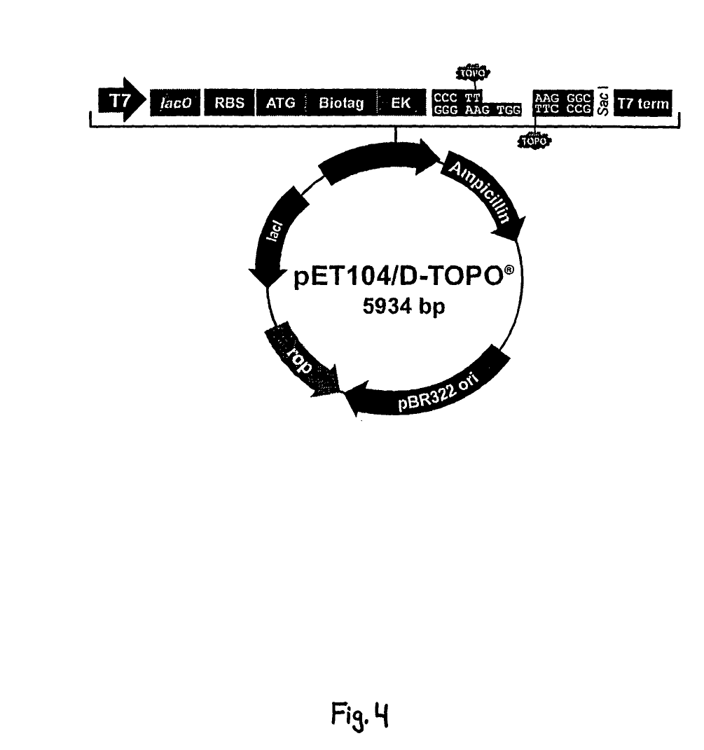

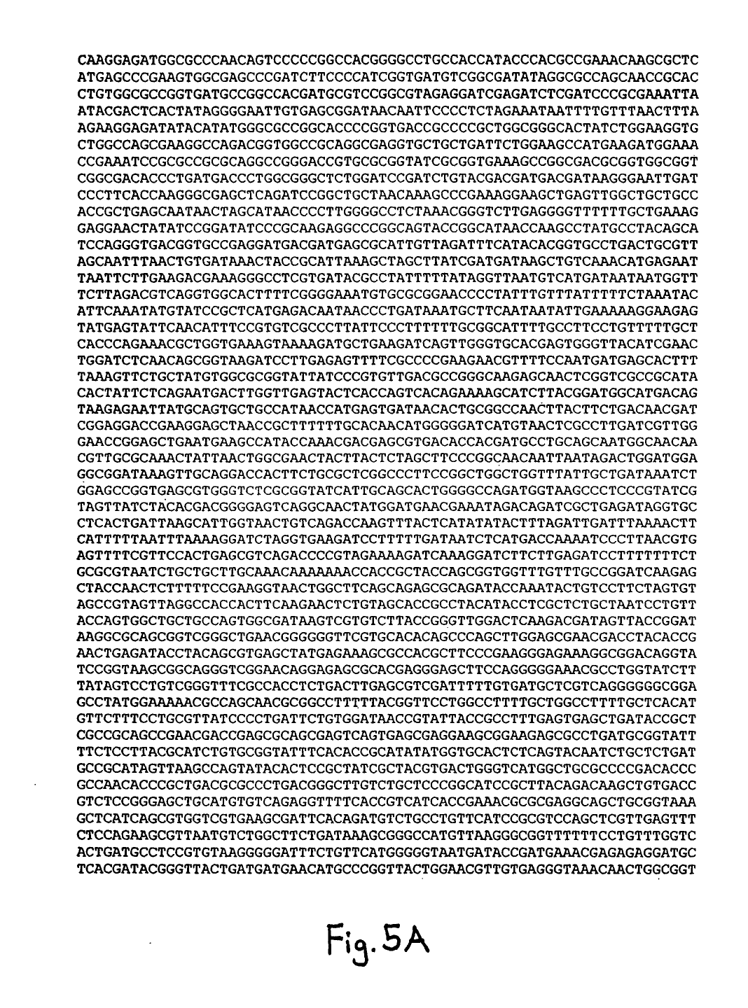

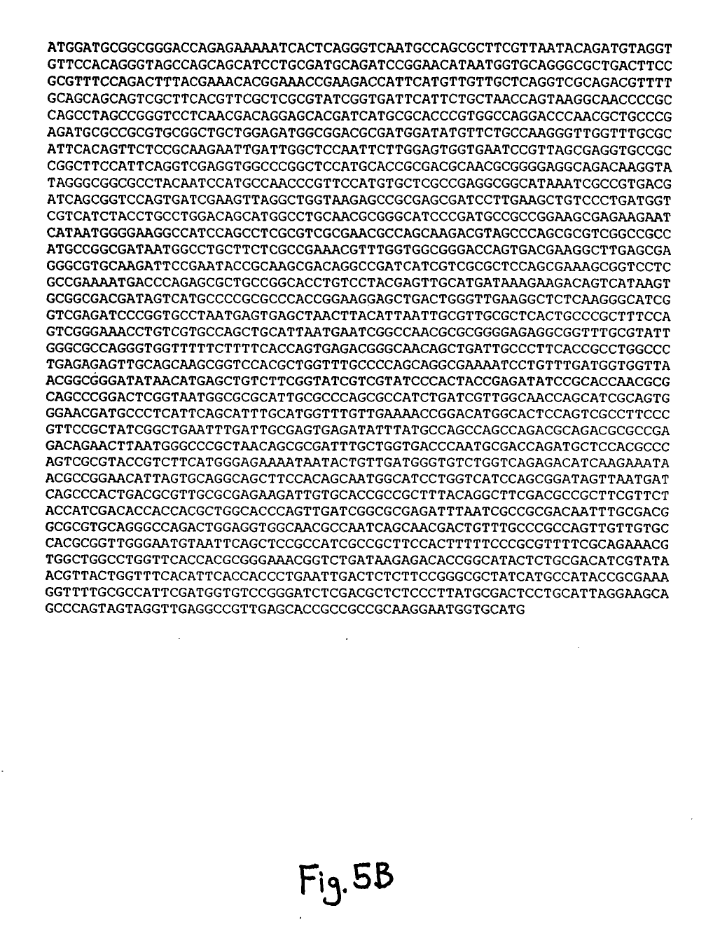

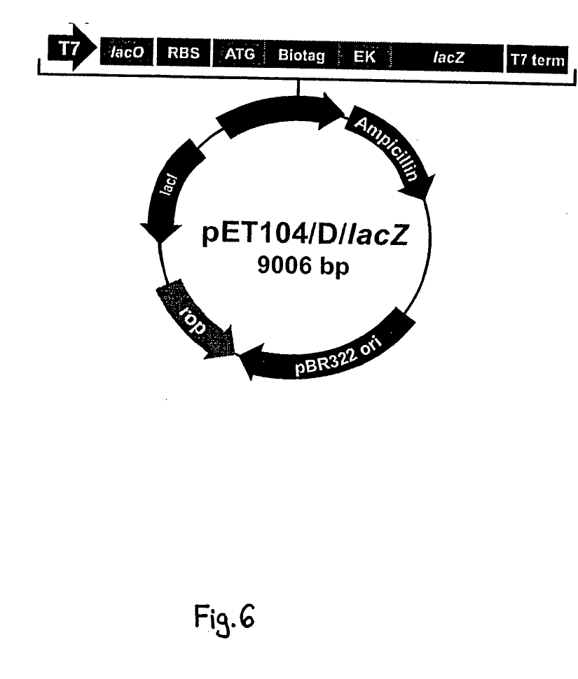

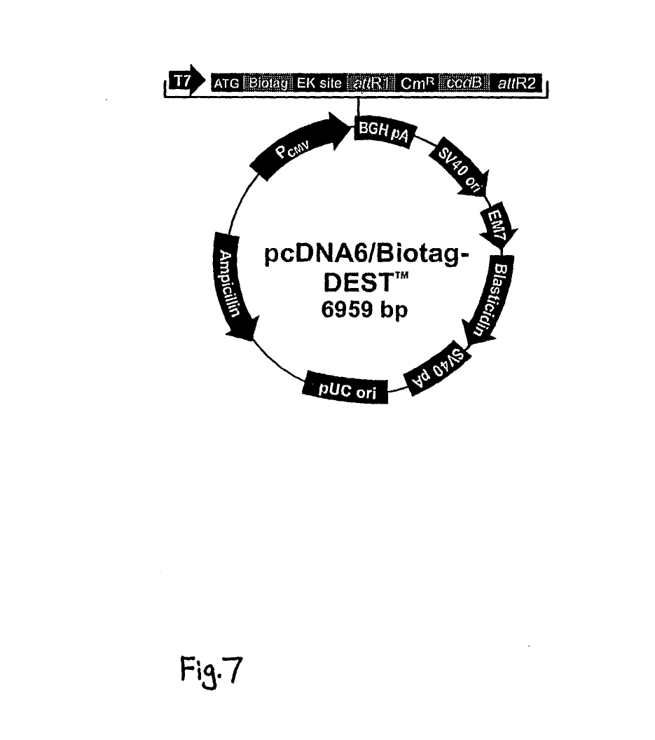

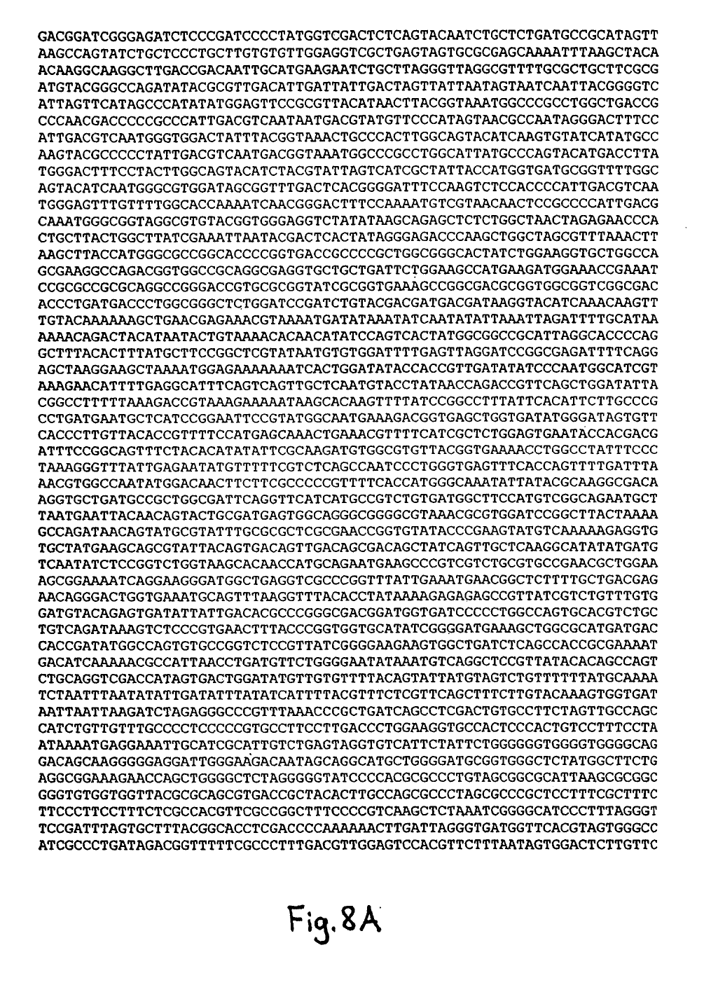

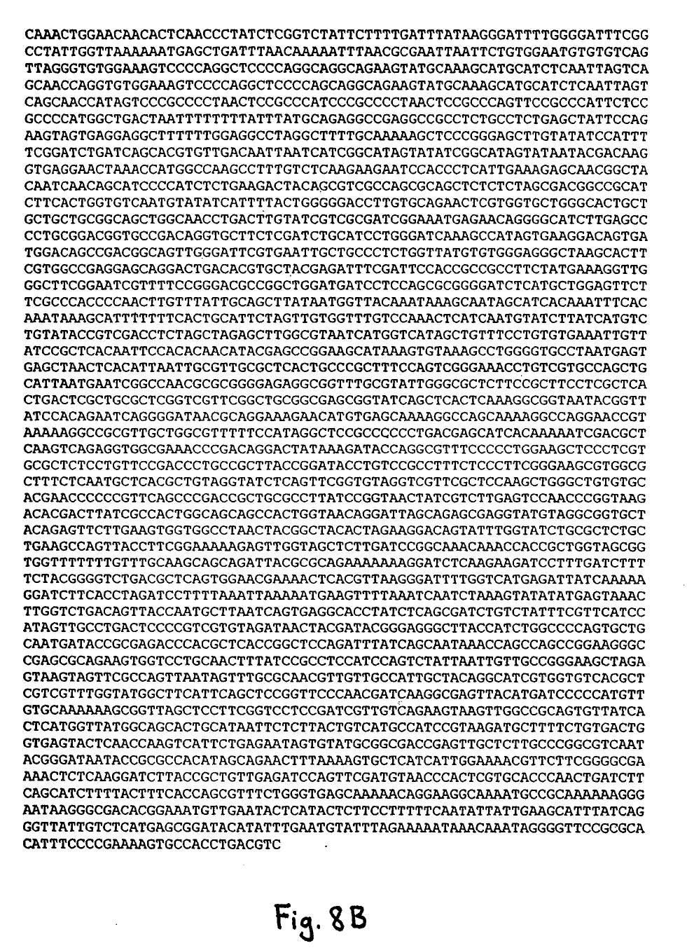

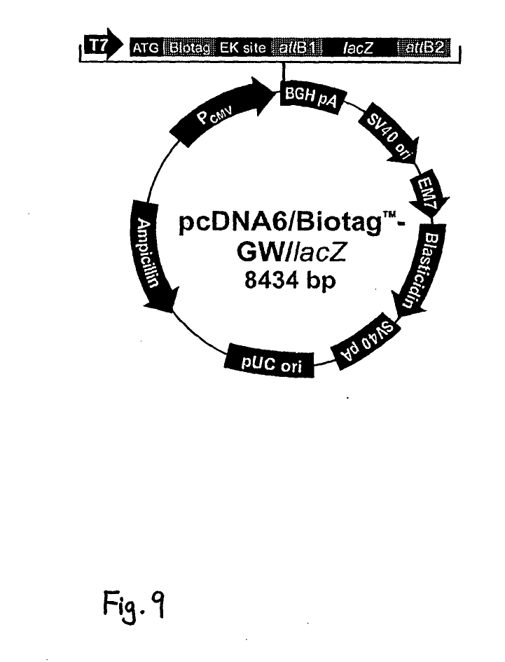

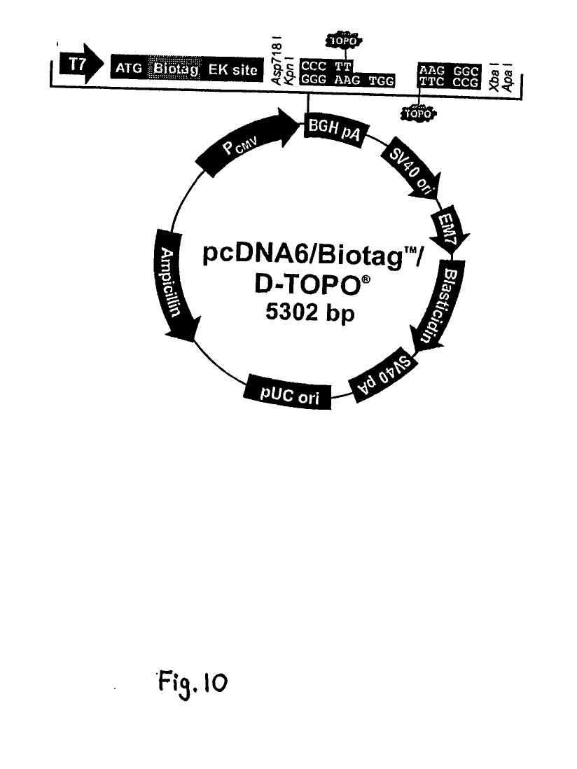

Exemplary vectors that are encompassed by the present invention include, e.g., pET104-DEST (SEQ ID NO:1) (FIG. 1), pET104/GW/lacZ (FIG. 2), pET104/D-TOPO (SEQ ID NO:2) (FIG. 3), pET104/D/lacZ (FIG. 4), pcDNA6/Biotag™-DEST (SEQ ID NO:3) (FIG. 5), pcDNA6/Biotag™-GW/lacZ (FIG. 6), pcDNA6/Biotag™/D-TOPO (SEQ ID NO:4) (FIG. 7), pcDNA6/Biotag™/lacZ (FIG. 8), pMT/Biotag™-DEST (SEQ ID NO:5) (FIG. 9), and pMT/Biotag™/GW-lacZ (FIG. 10).

The invention also encompasses nucleic acid molecules having nucleic acid sequences that are at least 80%, 85%, 90%, 91%, 92%, 93%, 94%, 95%, 96%, 97%, 98% or 99% identical to at least 25, 50, 100, 200, 300, 400, 500, 600, 700, 800, 900, 1000, 2000, 3000 or 4000 contiguous nucleotides of the exemplary vectors pET104-DEST (SEQ ID NO:1), pET104/D-TOPO (SEQ ID NO:2), pcDNA6/Biotag™-DEST (SEQ ID NO:3), pcDNA6/Biotag™/D-TOPO (SEQ ID NO:4) and pMT/Biotag™-DEST (SEQ ID NO:5). The invention also encompasses nucleic acid molecules comprising one or more nucleic acid sequences which encode an amino acid sequence tag, wherein said one or more nucleic acid sequences are at least 80%, 85%, 90%, 91%, 92%, 93%, 94%, 95%, 96%, 97%, 98% or 99% identical to at least 25, 50, 75, 100, 125, 150, 175 or 200 contiguous nucleotides of any one of SEQ ID Nos:11-15.

By a nucleic acid molecule having a nucleotide sequence at least, for example, 80% “identical” to a reference nucleotide sequence it is intended that the nucleotide sequence of the nucleic acid molecule is identical to the reference sequence except that the nucleotide sequence may include up to 20 nucleotide alterations per each 100 nucleotides of the nucleotide sequence of the reference nucleic acid molecule. In other words, to obtain a nucleic acid molecule having a nucleotide sequence at least 80% identical to a reference nucleotide sequence, up to 20% of the nucleotides in the reference sequence may be deleted or substituted with another nucleotide, or a number of nucleotides, up to 20% of the total nucleotides in the reference sequence, may be inserted into the reference sequence. These alterations of the reference sequence may occur, e.g., at the 5′ or 3′ ends of the reference nucleotide sequence and/or anywhere between those terminal positions, interspersed either individually among nucleotides in the reference sequence and/or in one or more contiguous groups within the reference sequence.

As a practical matter, whether any particular nucleic acid molecule is at least 80%, 85%, 90%, 91%, 92%, 93%, 94%, 95%, 96%, 97%, 98% or 99% identical to, for instance, a specified number of contiguous nucleotides of the nucleotide sequences shown in SEQ ID NOs:1-5 and 11-15 can be determined conventionally using known computer programs such as the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman, Advances in Applied Mathematics 2: 482-489 (1981), to find the best segment of homology between two sequences. When using Bestfit or any other sequence alignment program to determine whether a particular sequence is, for instance, 95% identical to a reference sequence according to the present invention, the parameters are set, of course, such that the percentage of identity is calculated over the full length of the reference nucleotide sequence and that gaps in homology of up to 5% of the total number of nucleotides in the reference sequence are allowed.

A preferred method for determining the best overall match between a query sequence (a sequence of the present invention) and a subject sequence, also referred to as a global sequence alignment, can be determined using the FASTDB computer program based on the algorithm of Brutlag et al., Comp. Appl. Biosci. 6:237-245 (1990). In a sequence alignment, the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k-tuple=4, Mismatch Penalty=1, Joining Penalty=30, Randomization Group Length=0, Cutoff Score=1, Gap Penalty=5, Gap Size Penalty=0.05, Window Size=500 or the length of the subject nucleotide sequence, whichever is shorter.

If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by the results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence are calculated for the purposes of manually adjusting the percent identity score.

For example, a 90 base subject sequence is aligned to a 100 base query sequence to determine percent identity. The deletions occur at the 5′ end of the subject sequence and, therefore, the FASTDB alignment does not show a match/alignment of the first 10 bases at the 5′ end. The 10 unpaired bases represent 10% of the sequence (number of bases at the 5′ and 3′ ends not matched/total number of bases in the query sequence), so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal, so that there are no bases on the 5′ or 3′ ends of the subject sequence which are not matched/aligned with the query. In this case, the percent identity calculated by FASTDB is not manually corrected. Once again, only bases 5′ and 3′ of the subject sequence which are not matched/aligned with the query sequence are manually corrected for. No other manual corrections are to be made for the purposes of the present invention.

The invention also includes host cells comprising any of the nucleic acid molecules and/or vectors described herein. As used herein, a host cell is any prokaryotic or eukaryotic organism that is a recipient of a replicable expression vector, cloning vector or any nucleic acid molecule. As used herein, the terms “host,” “host cell,” “recombinant host” and “recombinant host cell” may be used interchangeably. Representative host cells that may be used with the invention include, but are not limited to, bacterial cells, yeast cells, plant cells and animal cells. Preferred bacterial host cells include Escherichia spp. cells (particularly E. coli cells and most particularly E. coli strains DH10B, Stbl2, DH5, DB3, DB3.1 (preferably E. coli LIBRARY EFFICIENCY® DB3.1™ Competent Cells; Invitrogen Corporation, Carlsbad, Calif.), DB4 and DB5 (see U.S. application Ser. No. 09/518,188, filed Mar. 2, 2000, the disclosure of which is incorporated by reference herein in its entirety), Bacillus spp. cells (particularly B. subtilis and B. megaterium cells), Streptomyces spp. cells, Erwinia spp. cells, Klebsiella spp. cells, Serratia spp. cells (particularly S. marcessans cells), Pseudomonas spp. cells (particularly P. aeruginosa cells), and Salmonella spp. cells (particularly S. typhimurium and S. typhi cells). Preferred animal host cells include insect cells (most particularly Drosophila melanogaster cells, Spodoptera frugiperda Sf9 and Sf21 cells and Trichoplusa High-Five cells), nematode cells (particularly C. elegans cells), avian cells, amphibian cells (particularly Xenopus laevis cells), reptilian cells, and mammalian cells (most particularly NIH3T3, CHO, COS, VERO, BHK and human cells). Preferred yeast host cells include Saccharomyces cerevisiae cells and Pichia pastoris cells. These and other suitable host cells are available commercially, for example from Invitrogen Corporation (Carlsbad, Calif.), American Type Culture Collection (Manassas, Va.), and Agricultural Research Culture Collection (NRRL; Peoria, Ill.).

The nucleic acid molecules and/or vectors of the invention may be introduced into host cells using well known techniques of infection, transduction, electroporation, transfection, and transformation. The nucleic acid molecules and/or vectors of the invention may be introduced alone or in conjunction with other the nucleic acid molecules and/or vectors and/or proteins, peptides or RNAs. Alternatively, the nucleic acid molecules and/or vectors of the invention may be introduced into host cells as a precipitate, such as a calcium phosphate precipitate, or in a complex with a lipid. Electroporation also may be used to introduce the nucleic acid molecules and/or vectors of the invention into a host. Likewise, such molecules may be introduced into chemically competent cells such as E. coli. If the vector is a virus, it may be packaged in vitro or introduced into a packaging cell and the packaged virus may be transduced into cells. Hence, a wide variety of techniques suitable for introducing the nucleic acid molecules and/or vectors of the invention into host cells are well known and routine to those of skill in the art. Such techniques are reviewed at length, for example, in Sambrook, J., et al., Molecular Cloning, a Laboratory Manual, 2nd Ed., Cold Spring Harbor, N.Y.: Cold Spring Harbor Laboratory Press, pp. 16.30-16.55 (1989), Watson, J. D., et al., Recombinant DNA, 2nd Ed., New York: W. H. Freeman and Co., pp. 213-234 (1992), and Winnacker, E.-L., From Genes to Clones, New York: VCH Publishers (1987), which are illustrative of the many laboratory manuals that detail these techniques and which are incorporated by reference herein in their entireties for their relevant disclosures.

The present invention also includes methods of producing a polynucleotide construct that encodes a fusion protein that comprises one or more amino acid sequence tags. Such methods may be accomplished in vivo (e.g., within a cell) or in vitro (outside a cell).

According to one embodiment, the invention includes a method of producing a polynucleotide construct that encodes a fusion protein that comprises one or more amino acid sequence tags, said method comprising: (a) obtaining a first nucleic acid molecule comprising (i) a nucleotide sequence of interest and (ii) at least a first recombination site; (b) obtaining a second nucleic acid molecule comprising (i) one or more nucleic acid sequences which encode one or more amino acid sequence tags, and (ii) at least a second recombination site; and (c) combining said first nucleic acid molecule with said second nucleic acid molecule under conditions sufficient to cause recombination of at least said first and second recombination sites thereby producing a polynucleotide construct that encodes a fusion protein that comprises one or more amino acid sequence tags.

In certain embodiments, the methods of the invention comprise: (a) obtaining a first nucleic acid molecule comprising a nucleotide sequence of interest flanked by at least a first and at least a second recombination sites that do not recombine with each other; (b) obtaining a second nucleic acid molecule comprising: (i) at least a third and fourth recombination sites that do not recombine with each other; and (ii) one or more nucleic acid sequences which encode one or more amino acid sequence tags; and (c) contacting said first nucleic acid molecule with said second nucleic acid molecule under conditions favoring recombination between said first and third and between said second and fourth recombination sites, thereby producing a product polynucleotide construct; wherein said product polynucleotide construct encodes a fusion protein comprising: (i) said amino acid sequence tag; and (ii) the amino acid sequence encoded by said nucleotide acid sequence of interest.

In other embodiments, the methods of the invention comprise: (a) obtaining a first nucleic acid molecule comprising a nucleotide sequence of interest; (b) obtaining a second nucleic acid molecule comprising at least two topoisomerase recognition sites, at least one topoisomerase, and at least one nucleic acid sequence which encodes one or more amino acid sequence tags; (c) mixing said first nucleic acid molecule with said second nucleic acid molecule; and (d) incubating said mixture under conditions such that said first nucleic acid molecule is inserted into said second nucleic acid molecule between said at least two topoisomerase recognition sites, thereby producing a product polynucleotide construct; wherein said product polynucleotide construct encodes a fusion protein comprising: (i) said amino acid sequence tag; and (ii) the amino acid sequence encoded by said nucleotide sequence of interest.

In other embodiments, the methods of the invention comprise: (a) obtaining a first nucleic acid molecule comprising a nucleotide sequence of interest; (b) obtaining a second nucleic acid molecule comprising (i) at least a first topoisomerase recognition site flanked by (ii) at least a first recombination site, and (iii) at least a second topoisomerase recognition site flanked by (iv) at least a second recombination site, wherein said first and second recombination sites do not recombine with each other, and (v) at least one topoisomerase; (c) obtaining a third nucleic acid molecule comprising: (i) at least a third and fourth recombination sites that do not recombine with each other; and (ii) one or more nucleic acid sequences which encode one or more amino acid sequence tags; (d) mixing said first nucleic acid molecule with said second nucleic acid molecule; (e) incubating said mixture under conditions such that said first nucleic acid molecule is inserted into said second nucleic acid molecule between said at least two topoisomerase recognition sites, thereby producing a first product polynucleotide construct; (f) contacting said first product polynucleotide construct with said third nucleic acid molecule under conditions favoring recombination between said first and third and between said second and fourth recombination sites, thereby producing a second product polynucleotide construct; wherein said second product polynucleotide construct encodes a fusion protein comprising: (i) said amino acid sequence tag; and (ii) the amino acid sequence encoded by said nucleotide sequence of interest.

In particular embodiments of the invention, one or more of the nucleic acid molecules that are used in the practice of the methods will further comprise a nucleic acid sequence that encodes an amino acid sequence that is capable of being cleaved by one or more proteases, and wherein the product polynucleotide constructs encode a fusion protein comprising: (i) said amino acid sequence that is capable of being cleaved by one or more proteases, flanked on one side by (ii) an amino acid sequence tag, and on the other side by (iii) the amino acid sequence encoded by a nucleotide sequence of interest. Any of the amino acid sequences that are capable of being cleaved by one or more proteases, as described elsewhere herein, can be used with the methods of the invention. In a preferred embodiment, the amino acid sequence that is capable of being cleaved by one or more proteases is an amino acid sequence that is capable of being cleaved by enterokinase.

The methods of the invention involve the use of nucleic acid molecules comprising one or more nucleic acid sequences which encode one or more amino acid sequence tags. Any of the nucleic acid sequences, described elsewhere herein, which encode an amino acid sequence tag, can be used in the context of the methods of the invention. In certain embodiments of the invention, the amino acid sequence tag is an amino acid sequence that is capable of being post-translationally modified. For example, the amino acid sequence tag may be an amino acid sequence that is capable of being biotinylated.

Any of the nucleic acid molecules, vectors, and host cells described herein, including any variations or modifications of such nucleic acid molecules vectors, and host cells, can be included in the practice of the methods of the invention. The nucleic acid molecules that are used in the practice of the methods of the invention may be linear, or circular. If a linear nucleic acid molecule is used, the ends of the molecule may be blunt ended or, alternatively, may have one or more overhang ends. The nucleic acid molecules that are used in the practice of the methods of the invention may be PCR products.

The methods of the invention may further comprise inserting a product polynucleotide construct into a host cell.

In certain embodiments, the methods of the invention comprise contacting a first nucleic acid molecule comprising a first and a second recombination site with a second nucleic acid molecule comprising a third and a fourth recombination site under conditions favoring recombination between a first and third and between a second and fourth recombination sites.

Exemplary recombination sites included within the nucleic acid molecules that are used in the practice of the methods of the invention include, but are not limited to, (a) attB sites, (b) attP sites, (c) attL sites, (d) attR sites, (e) lox sites, (f) psi sites, (g) dif sites, (h) cer sites, (i) frt sites, and mutants, variants, and derivatives of the recombination sites of (a), (b), (c), (d), (e), (f), (g), (h), or (i) which retain the ability to undergo recombination.

In particular embodiments, said first and said second nucleic acid molecules are combined in the presence of at least one recombination protein. Exemplary recombination proteins that can be used in the methods of the invention include, e.g., Cre, Int, IHF, Xis, Fis, Hin, Gin, Cin, Tn3 resolvase, TndX, XerC and XerD.

Methods for combining nucleic acid molecules by recombination at particular sites are known in the art. Such methods include, e.g., recombinational cloning methods.

Cloning systems that utilize recombination at defined recombination sites have been previously described in U.S. Pat. Nos. 5,888,732, 6,143,557, 6,171,861, 6,270,969, and 6,277,608, and in commonly owned, co-pending U.S. application Ser. No. 10/005,876 (filed Dec. 7, 2001), which are specifically incorporated herein by reference. In brief, the Gateway™ Cloning System, described in this application and the applications referred to in the related applications section, utilizes vectors that contain at least one and preferably at least two different site-specific recombination sites based on the bacteriophage lambda system (e. g., att1 and att2) that are mutated from the wild type (att0) sites. Each mutated site has a unique specificity for its cognate partner att site of the same type (for example attB1 with attP1, or attL1 with attR1) and will not cross-react with recombination sites of the other mutant type or with the wild-type att0 site. Nucleic acid fragments flanked by recombination sites are cloned and subcloned using the Gateway™ system by replacing a selectable marker (for example, ccdB) flanked by att sites on the recipient plasmid molecule, sometimes termed the Destination Vector. Desired clones are then selected by transformation of a ccdB sensitive host strain and positive selection for a marker on the recipient molecule. Similar strategies for negative selection (e.g., use of toxic genes) can be used in other organisms such as thymidine kinase (TK) in mammals and insects.

Mutating specific residues in the core region of the att site can generate a large number of different att sites. As with the att1 and att2 sites utilized in Gateway™, each additional mutation potentially creates a novel att site with unique specificity that will recombine only with its cognate partner att site bearing the same mutation and will not cross-react with any other mutant or wild-type att site. Novel mutated att sites (e. g., attB 1-10, attP 1-10, attR 1-10 and attL 1-10) are described in International Patent Application PCT/US00/05432, which is specifically incorporated herein by reference. Other recombination sites having unique specificity (i.e., a first site will recombine with its corresponding site and will not recombine or not substantially recombine with a second site having a different specificity) may be used to practice the present invention. Examples of suitable recombination sites include, but are not limited to, loxP sites and derivatives such as loxP5 11 (see U.S. Pat. No. 5,851,808), frt sites and derivatives, dif sites and derivatives, psi sites and derivatives and cer sites and derivatives. The present invention provides novel methods using such recombination sites to join or link multiple nucleic acid molecules or segments and more specifically to clone such multiple segments into one or more vectors containing one or more recombination sites (such as any Gateway™ Vector including Destination Vectors).

In certain embodiments, the methods of the invention comprise (a) mixing a first nucleic acid molecule with a second nucleic acid molecule, said second nucleic acid molecule comprising at least two topoisomerase recognition sites and at least one topoisomerase, and (b) incubating the mixture under conditions such that said first nucleic acid molecule is inserted into said second nucleic acid molecule between said at least two topoisomerase recognition sites.

Methods for inserting a first nucleic acid molecule into a second nucleic acid molecule between topoisomerase recognition sites thereby producing a product polynucleotide construct, are known in the art. Exemplary methods are known in the art as Topoisomerase cloning, TOPO® cloning, and Directional TOPO®) cloning. As used herein, the term “topoisomerase-mediated cloning” is intended to mean any method of combining two or more nucleic acid molecules using at least one topoisomerase recognition site on one or more of the nucleic acid molecules and one or more topoisomerase. Exemplary methods are described in commonly owned, co-pending U.S. application Ser. No. 10/005,876 (filed Dec. 7, 2001), the disclosure of which is incorporated herein by reference in its entirety.

A method for generating a product polynucleotide construct using topoisomerase cloning can be performed, for example, by contacting a first nucleic acid molecule having a first end and a second end, wherein, at the first end or second end or both, the first nucleic acid molecule has a topoisomerase recognition site (or cleavage product thereof) at or near the 3′ terminus; at least a second nucleic acid molecule having a first end and a second end, wherein, at the first end or second end or both, the at least second double stranded nucleotide sequence has a topoisomerase recognition site (or cleavage product thereof) at or near a 3′ terminus; and at least one site specific topoisomerase (e.g., a type IA and/or a type IB topoisomerase), under conditions such that all components are in contact and the topoisomerase can effect its activity.

In one embodiment, the method is performed by contacting a first nucleic acid molecule and a second (or other) nucleic acid molecule, each of which has a topoisomerase recognition site, or a cleavage product thereof, at the 3′ termini or at the 5′ termini of two ends to be covalently linked. In another embodiment, the method is performed by contacting a first nucleic acid molecule having a topoisomerase recognition site, or cleavage product thereof, at the 5′ terminus and the 3′ terminus of at least one end, and a second (or other) nucleic acid molecule having a 3′ hydroxyl group and a 5′ hydroxyl group at the end to be linked to the end of the first nucleic acid molecule containing the recognition sites. As disclosed herein, the methods can be performed using any number of nucleic acid molecules having various combinations of termini and ends.

Method of the invention may involve the use of nucleic acid molecule that comprises at least one topoisomerase. The topoisomerase may be, e.g., a type I topoisomerase. More specifically, the type I topoisomerase may be a type IB topoisomerase. Where a type IB topoisomerase is used, the type IB topoisomerase may be a topoisomerase selected, e.g., from the group consisting of eukaryotic nuclear type I topoisomerase and a poxvirus topoisomerase. Poxvirus topoisomerases may be produced by or isolated from a virus selected from the group consisting of vaccinia virus, Shope fibroma virus, ORF virus, fowlpox virus, molluscum contagiosum virus and Amsacta moorei entomopoxvirus.

The present invention includes methods for producing a polynucleotide construct that encodes a fusion protein that comprises one or more amino acid sequence tags, using, for example, recombinational cloning or topoisomerase-mediated cloning. The methods of the invention may also involve the use of a combination of recombinational cloning and topoisomerase-mediated cloning.

For example, the invention includes methods comprising the successive use of one or more recombinational cloning steps followed by one or more topoisomerase-mediated cloning steps. Alternatively, the invention also includes methods comprising the successive use of one or more topoisomerase-mediated cloning steps followed by one or more recombinational cloning steps. Alternatively, the invention includes methods comprising the use of recombinational cloning and topoisomerase-mediated cloning in the same cloning step.

One example of the use of topoisomerase-mediated cloning followed by recombinational cloning to produce a polynucleotide construct that encodes a fusion protein capable of being post-translationally modified or that is capable of being recognized by an antibody (or fragment thereof) or other specific binding reagent, is as follows. A first nucleic acid molecule comprising a nucleotide sequence of interest is mixed with a second nucleic acid molecule comprising: (i) at least a first topoisomerase recognition site flanked by (ii) at least a first recombination site, and (iii) at least a second topoisomerase recognition site flanked by (iv) at least a second recombination site, wherein said first and second recombination sites do not recombine with each other, and (v) at least one topoisomerase. The mixture is incubated under conditions such that said first nucleic acid molecule is inserted into said second nucleic acid molecule between said at least two topoisomerase recognition sites, thereby producing a first product polynucleotide construct. The first product polynucleotide construct is then brought into contact with a third nucleic acid molecule comprising: (i) at least a third and fourth recombination sites that do not recombine with each other and (ii) one or more nucleic acid sequences which encode one or more amino acid sequence tags. The first product polynucleotide construct is contacted with said third nucleic acid molecule under conditions favoring recombination between said first and third and between said second and fourth recombination sites, thereby producing a second product polynucleotide construct. According to this exemplary method, said second polynucleotide construct will encode a fusion protein comprising: (i) said amino acid sequence tag, and (ii) the amino acid sequence encoded by said nucleotide sequence of interest.

Another example of the use of topoisomerase-mediated cloning followed by recombinational cloning to produce a polynucleotide construct that encodes a fusion protein that comprises an amino acid sequence tag, is as follows: A first nucleic acid molecule comprising a nucleotide sequence of interest is mixed with a second nucleic acid molecule comprising: (i) at least a first topoisomerase recognition site flanked by (ii) at least a first recombination site, and (iii) at least a second topoisomerase recognition site flanked by (iv) at least a second recombination site, wherein said first and second recombination sites do not recombine with each other, (v) one or more nucleic acid sequences which encode one or more amino acid sequence tags, and (vi) at least one topoisomerase. The mixture is incubated under conditions such that said first nucleic acid molecule is inserted into said second nucleic acid molecule between said at least two topoisomerase recognition sites, thereby producing a first product polynucleotide construct. The first product polynucleotide construct is then brought into contact with a third nucleic acid molecule comprising: (i) at least a third and fourth recombination sites that do not recombine with each other. The first product polynucleotide construct is contacted with said third nucleic acid molecule under conditions favoring recombination between said first and third and between said second and fourth recombination sites, thereby producing a second product polynucleotide construct. According to this exemplary method, said second polynucleotide construct will encode a fusion protein comprising: (i) said amino acid sequence tag, and (ii) the amino acid sequence encoded by said nucleotide sequence of interest.

The invention also includes host cells comprising one or more polynucleotide construct that encodes a fusion protein, e.g., a fusion protein that comprises one or more amino acid sequence tags, wherein said polynucleotide construct is produced according to a method of the invention.

The nucleic acid molecules and methods of the invention can be used, e.g., to produce a fusion protein comprising one or more amino acid sequence tags, and an amino acid sequence encoded by a nucleic acid sequence of interest. Accordingly, the present invention includes methods for producing fusion proteins comprising one or more amino acid tags. The methods of the invention can be used to produce fusion proteins in vitro or in vivo. When in vivo methods are used, the fusion protein can be produced in either eukaryotic or prokaryotic cells. Methods for producing proteins in vivo and in vitro are well known in the art.

According to certain embodiments, the invention provides methods for producing a fusion protein that comprises one or more amino acid sequence tags, said methods comprising: (a) obtaining a host cell comprising a polynucleotide construct that encodes a fusion protein that comprises one or more amino acid sequence tags, said polynucleotide construct produced according to a method of the invention; and (b) culturing said host cell under conditions wherein said fusion protein is produced by said host cell. The precise conditions for producing a fusion protein in a host cell will vary, depending on the host cell used and the nature of the fusion protein being produced, and will be appreciated by those of ordinary skill in the art. In certain embodiments, the methods of the invention further comprise culturing said host cell under conditions wherein said fusion protein is post-translationally modified in said host cell. For example, the fusion protein may be biotinylated in said host cell.

In yet other embodiments, the methods may further comprise causing said fusion protein to be released from said host cell or treating said host cell such that said fusion protein is released from said host cell; and (b) contacting said fusion protein with a detecting composition comprising a molecule that is capable of interacting with said fusion protein. In an exemplary embodiment, the fusion protein will be a post-translationally modified fusion protein, e.g., a biotinylated fusion protein, and said detecting composition will comprise avidin or an avidin analogue (including e.g., streptavidin).

Methods for treating a host cell such that a protein, produced therein, is released from said host cell, are well known in the art and include, e.g., chemical disruption of the cell and physical disruption of the cell including, e.g., boiling, freezing, grinding, and combinations of chemical and physical disruption of the cell. Such methods include producing a protein extract from said host cell.

Details regarding the production and detection of fusion proteins that comprise one or more amino acid sequence tags, in general, are known in the art. (See, e.g., Parrott, M. B. and Barry, M. A., Biochem. Biophys. Res. Comm. 281:993-1000 (2001), Parrott, M. B. and Barry, M. A., Mol. Ther. 1:96-104 (2000), U.S. Pat. No. 5,252,466, and references cited therein).

The invention also includes methods for purifying, isolating or concentrating fusion proteins that are produced using the compositions and methods of the invention. In one embodiment, the invention includes methods for purifying, isolating or concentrating fusion proteins that have been post-translationally modified by a post-translational modification reaction, either in vivo or in vitro. In another embodiment, the invention includes methods for purifying, isolating or concentrating fusion proteins that comprise an amino acid sequence that is capable of being recognized by one or more antibody (or fragment thereof) or other specific reagents.

In an exemplary embodiment, the fusion proteins of the invention are purified, isolated or concentrated by bringing the fusion proteins into contact with a composition that is capable of interacting with the amino acid sequence tag and/or with a molecular entity that is attached to the amino acid sequence tag. Such compositions that interact specifically with an amino acid sequence tag include, e.g., “detecting compositions.” As used herein, the term “detecting composition” is intended to mean any composition comprising a molecule that is capable of interacting with an amino acid sequence tag or with a molecular entity that is attached to an amino acid sequence tag, e.g., a molecule that is capable of interacting with a molecular entity that was attached to the amino acid sequence tag in a post-translational modification reaction. Such molecules that interact with amino acid sequence tags include, e.g., proteins and polypeptides, including, e.g., antibodies (or fragments thereof including fab fragments, fc fragments, etc) specific for the amino acid sequence tag. Particular exemplary molecules that can be attached to a detecting composition include avidin, streptavidin, and derivatives and analogs of those two compounds, as well as metal compounds (e.g., arsenites and thallium) that bind to dithiols such as lipoic acid (U.S. Pat. No. 5,252,466), and antibodies (or fragments thereof) specific for epitopes such as, e.g., the FLAG epitope, the Myc epitope, the HA epitope, etc.

Detecting compositions may further comprise a surface (including, e.g., a solid and semi-solid surface), a matrix or a substrate, to which the molecule that is capable of interacting with particular amino acid sequence tag (or molecular entity attached thereto) is attached. Exemplary surfaces, matrices and substrates include, e.g., agarose beads, plastic beads, microscope coverslips, microscope slides, magnetic beads, glass beads or planar surfaces. The attachment may be, e.g., covalent or non-covalent. The types of surfaces, matrices and substrates to which a molecule that is capable of interacting with an amino acid sequence tag (or molecular entity attached thereto) may be attached are known in the art (see, e.g., Zou, H. et al., J. Biochem. Biophys. Methods 49:1-3:199-240 (2001), Zusman, R. and Zusman, I., J. Biochem. Biophys. Methods 49:1-3:175-187 (2001)). Exemplary detecting compositions include agarose beads to which avidin, streptavidin, or derivatives/analogs thereof, are attached.

In certain embodiments, the detecting composition may be used to identify, concentrate or purify a fusion protein by, e.g., mixing the detecting composition with a solution or composition comprising the fusion protein of interest, wherein the mixing takes place in batch (e.g., in a vessel such as a beaker, flask, bottle, test tube, petri dish, or other suitable container) or through a column containing the detecting composition. The detecting composition may alternatively be applied to a solution, to a cell (e.g., a permeablized cell), or to any other substance that is known to contain or suspected of containing the fusion protein of interest.

In certain embodiments, the fusion proteins of the invention will be post-translationally modified fusion proteins, e.g., fusion proteins that have been biotinylated at the amino acid sequence tag. The biotinylated fusion protein can be purified, isolated or concentrated from a mixture of other proteins and molecules by bringing the biotinylated fusion protein into contact with, e.g., a detecting composition comprising a molecule that specifically interacts with biotin. Such molecules include, e.g., avidin and avidin derivatives such as streptavidin. The detecting composition may further comprise a surface or support matrix that can be physically removed from a mixture of proteins and other molecules, e.g., agarose beads, or other equivalent beads.

In other embodiments, the fusion protein that is produced using the methods and compositions of the invention will comprise an amino acid sequence that is capable of being cleaved by one or more proteases, flanked on one side by an amino acid sequence tag, and on the other side by an amino acid sequence encoded by a nucleic acid sequence of interest. After purifying, isolating or concentrating such a fusion protein, the fusion protein can be treated with a protease to separate the amino acid sequence tag from the amino acid sequence encoded by a nucleic acid sequence of interest.

The invention also includes compositions or reaction mixtures comprising one or more nucleic acid molecule of the invention. The compositions or reaction mixtures may additionally comprise, one or more additional components selected from the group consisting of one or more topoisomerases, one or more host cells (e.g., host cells that may be competent for uptake of nucleic acid molecules) one or more recombination proteins, one or more vectors, one or more nucleotides, one or more primers, and one or more polypeptides having polymerase activity.

The invention also provides kits comprising the isolated nucleic acid molecules of the invention, which may optionally comprise one or more additional components selected from the group consisting of one or more topoisomerases, one or more recombination proteins, one or more vectors, one or more nucleotides, one or more primers, one or more polypeptides having polymerase activity, one or more host cells (e.g., host cells that may be competent for uptake of nucleic acid molecules), one or more antibody (or fragment thereof), and one or more detecting compositions, including, e.g., one or more support matrices complexed with avidin or an avidin analog.

It will be readily apparent to one of ordinary skill in the relevant arts that other suitable modifications and adaptations to the methods and applications described herein are obvious and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLE 1

### A Gateway™-Adapted Destination Vector for Cloning and Expression of Biotinylated Fusion Proteins in E. coli

This example describes the pET104-DEST expression vector (FIG. 1). pET104-DEST is a 7.6 kb vector adapted for use with the Gateway™ Technology, and is designed to allow for high-level, inducible expression of biotinylated recombinant fusion proteins in E. coli using the pET system. Biotinylated recombinant protein may then be easily detected or immobilized to a solid support for other downstream applications.

The pET system was originally developed by Studier and colleagues and takes advantage of the high activity and specificity of the bacteriophage T7 RNA polymerase to allow regulated expression of heterologous genes in E. coli from the T7 promoter (Rosenberg, A. H. et al., Gene 56:125-135 (1987); Studier, F. W. and Moffatt, B. A., J. Mol. Biol. 189:113-130 (1986); Studier, F. W. et al., Meth. Enzymol. 185:60-89 (1990)).

The pET104-DEST vector comprises the following elements:

(a) T7lac promoter for high-level, IPTG-inducible expression of the gene of interest in E. coli (Dubendorff, J. W., and Studier, F. W., J. Mol. Biol. 219:45-59 (1991); ); Studier, F. W. et al., Meth. Enzymol. 185:60-89 (1990));

(b) Biotag™ to allow biotinylation of the recombinant protein of interest for easy detection or use in other applications;

(c) Enterokinase (EK) recognition site for cleavage of the Biotag™ from the recombinant protein;

(d) Two recombination sites, attR1 and attR2, downstream of the CMV promoter for recombinational cloning of the gene of interest from an entry clone;

(e) Chloramphenicol resistance gene (CmR) located between the two attR sites for counterselection;

(f) The ccdB gene located between the attR sites for negative selection;

(g) lacI gene encoding the lac repressor to reduce basal transcription from the T7lac promoter in the pET104-DEST vector and from the lacUV5 promoter in the E. coli chromosome;

(h) Ampicillin resistance gene for selection in E. coli; and

(i) pBR322 origin for low-copy replication and maintenance of the plasmid in E. coli.

The control plasmid, pET104/GW/lacZ (FIG. 2), can be used as a positive control for expression in E. coli. pET104/GW/lacZ was generated using the Gateway LR recombination reaction between an entry clone containing the lacZ gene and pET104-DEST.

To recombine a gene of interest into pET104-DEST, an entry clone containing a gene of interest will be obtained. Details relating to choosing an entry vector and constructing an entry clone are available in the art (See, e.g., U.S. Pat. No. 6,270,969).

pET104-DEST is an N-terminal fusion vector and contains an ATG initiation codon. A Shine-Dalgarno ribosome binding site (RBS) is included upstream of the initiation. The gene of interest in the entry clone must: (a) be in frame with the N-terminal Biotag™ after recombination; and (b) contain a stop codon.

The entry clone will contain, e.g., attL sites flanking the gene of interest. Genes in an entry clone are transferred to the destination vector backbone by mixing the DNAs with, e.g., the Gateway LR Clonase Enzyme Mix. The resulting LR recombination reaction is then transformed into E. coli (e.g., TOP10 or DH5α-T1R) and the expression clone is selected using ampicillin. Recombination between the attR sites on the destination vector and the attL sites on the entry clone replaces the chloramphenicol (CmR) gene and the ccdB gene with the gene of interest and results in the formation of attB sites in the expression clone. Details for setting up the recombination reaction, transforming E. coli, and selecting for the expression clone, are available in the art.

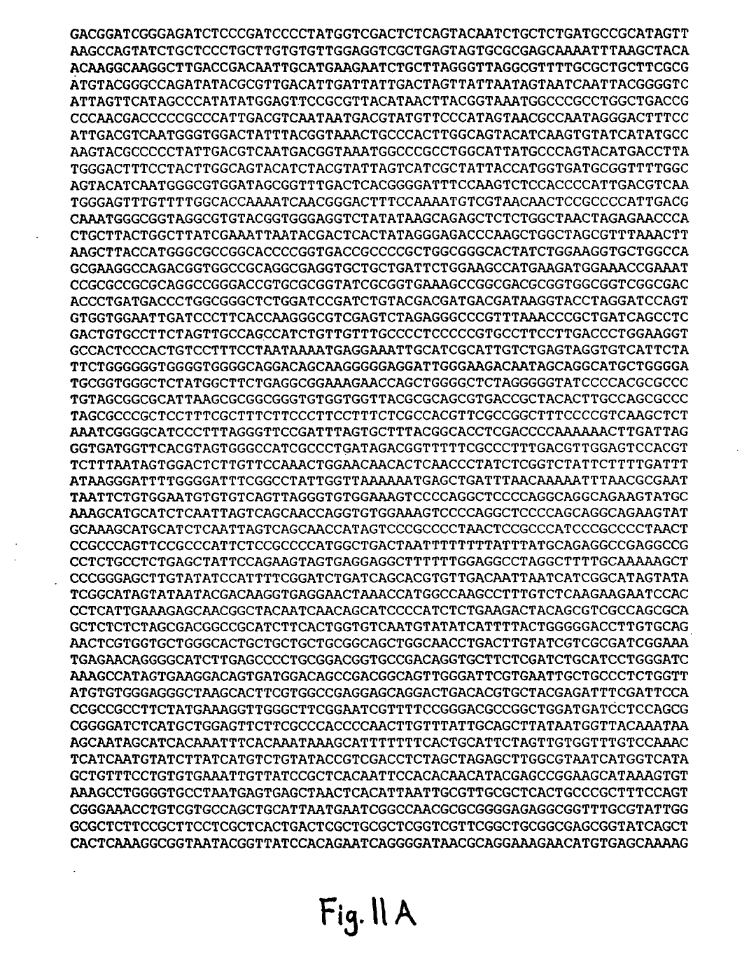

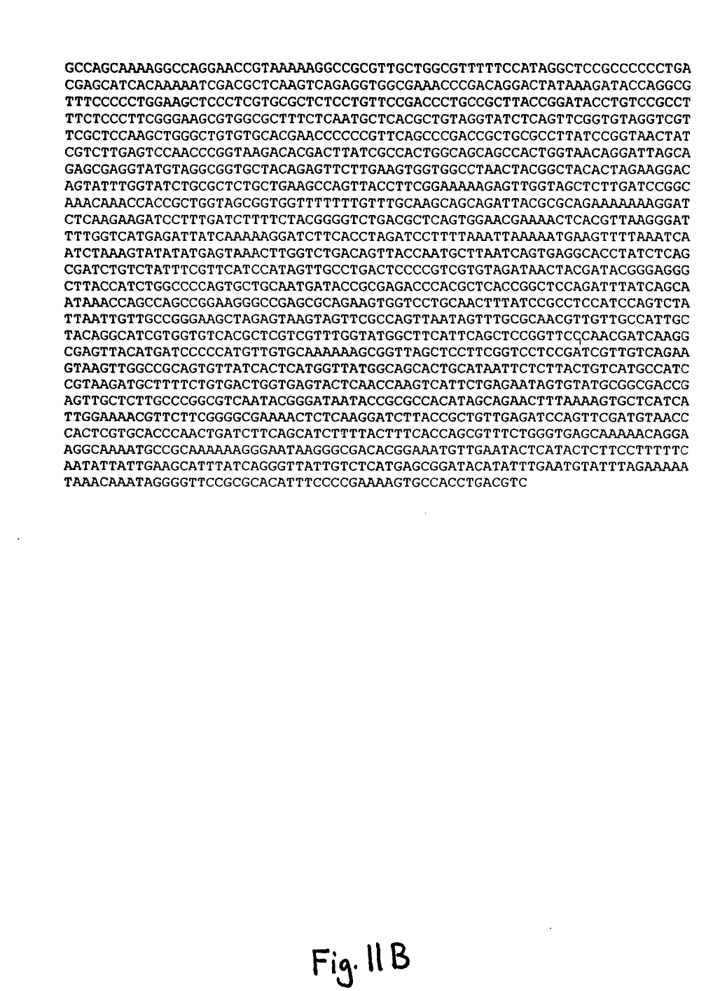

The recombination region of the expression clone resulting from pET104-DEST x entry clone is depicted in FIG. 11. Features of the recombination region are as follows:

(a) shaded regions correspond to those DNA sequences transferred from the entry clone into the pET104-DEST vector by recombination. Non-shaded regions are derived from the pET104-DEST vector;

(b) bases 568 and 2230 of the pET104-DEST sequence are marked.

(c) The biotin binding site is labeled with an asterisk (*).

The Expression clone can be confirmed following recombination. The ccdB gene mutates at a very low frequency, resulting in a very low number of false positives. True expression clones will be ampicillin-resistant and chloramphenicol-sensitive. Transformants containing a plasmid with a mutated ccdB gene will be both ampicillin- and chloramphenicol-resistant. To check a putative expression clone, transformants can be tested for growth on LB plates containing 30 μg/ml chloramphenicol. A true expression clone should not grow in the presence of chloramphenicol.

The expression construct may also be sequenced to confirm that the gene of interest is in frame with the Biotag™. The priming sites indicated in FIG. 11 can be used to sequence the insert.

Expression of the recombinant fusion protein can be induced by first transforming the expression clone into an appropriate E. coli strain for protein expression, e.g., BL21 cells. The transformant is then grown to mid-log in LB containing 100 μg/ml ampicillin or 50 μg/ml carbenicillin, and IPTG is added to a final concentration of 0.5-1 mM.

Expression of the recombinant fusion protein can be detected, e.g., by western blot analysis using, e.g., streptavidin-HRP or streptavidin-AP conjugates, or an antibody (or fragment thereof) specific for the protein of interest.

The recombinant fusion protein can then be purified. The presence of the N-terminal Biotag™ in pET104-DEST allows the recombinant fusion protein to be biotinylated. Once biotinylated, the recombinant fusion protein can be purified by taking advantage of the strong association between biotin and avidin (and its analogs including streptavidin). For example, streptavidin agarose-conjugated beads can be used to purify the recombinant fusion protein. Other streptavidin conjugates can also be used.

A streptavidin-agarose resin can be used for affinity purification of recombinant fusion proteins containing the Biotag™. The resin can be constructed by covalently linking streptavidin to cross-linked agarose beads via a 15-atom hydrophilic spacer arm specifically designed to reduce non-specific binding and to ensure optimal binding of biotinylated molecules. Streptavidin is bound to a final concentration of 2-3 mg streptavidin per ml of packed resin.

Recombinant fusion proteins may be purified with streptavidin-agarose under native or denaturing conditions. Methods for purifying biotinylated proteins are known in the art.

pET104-DEST contains an enterokinase (EK) recognition site to allow removal of the Biotag™ from the recombinant fusion protein, if desired. After digestion with enterokinase, 11 amino acids will remain at the N-terminus of the protein (see FIG. 11). Methods for digestion with enterokinase are known in the art.

### EXAMPLE 2

### Directional TOPO Cloning of Blunt-End PCR Products into a Vector for Biotinylated Expression in E. coli

This example describes directional TOPO cloning using the pET104/D-TOPO vector (FIG. 3).

pET104/D-TOPO is a 5.9 kb vector designed to facilitate rapid, directional TOPO cloning of blunt-end PCR products for regulated and biotinylated expression in E. coli. The pET104/D-TOPO vector comprises the following elements:

(a) T7lac promoter for high-level, IPTG-inducible expression of the gene of interest in E. coli (Dubendorff, J. W., and Studier, F. W., J. Mol. Biol. 219:45-59 (1991); ); Studier, F. W. et al., Meth. Enzymol. 185:60-89 (1990));

(b) Directional TOPO cloning site for rapid and efficient directional cloning of blunt-end PCR products;

(c) Biotag™ to allow biotinylation of the recombinant protein of interest for easy detection or use in other applications;

(d) Enterokinase (EK) recognition site for cleavage of the Biotag™ from the recombinant protein;

(e) lacI gene encoding the lac repressor to reduce basal transcription from the T7lac promoter in the pET104/D-TOPO vector and from the lacUV5 promoter in the E. coli chromosome;

(f) Ampicillin resistance gene for selection in E. coli; and

(g) pBR322 origin for low-copy replication and maintenance of the plasmid in E. coli.

The control plasmid, pET104/D/lacZ (FIG. 4), can be used as a positive control for expression in E. coli. The gene encoding β-galactosidase was directionally TOPO cloned into the pET104/D-TOPO vector.

Topoisomerase I from Vaccinia virus binds to duplex DNA at specific sites and cleaves the phosphodiester backbone after 5′-CCCTT in one strand (Shuman, S., Proc. Natl. Acad. Sci. USA 88:10104-10108 (1991)). The energy from the broken phosphodiester backbone is conserved by formation of a covalent bond between the 3′ phosphate of the cleaved strand and a tyrosyl residue (Tyr-274) of topoisomerase I. The phospho-tyrosyl bond between the DNA and enzyme can subsequently be attacked by the 5′ hydroxyl of the original cleaved strand, reversing the reaction and releasing topoisomerase (Shuman, S., J. Biol. Chem. 269:32678-32684 (1994)). TOPO cloning exploits this reaction to efficiently clone PCR products.

Directional joining of double-strand DNA using TOPO-charged oligonucleotides occurs by adding a 3′ single-stranded end (overhang) to the incoming DNA (Cheng, C. and Shuman, S., Mol. Cell. Biol. 20:8059-8068 (2000)). This single-stranded overhang is identical to the 5′ end of the TOPO-charged DNA fragment. A 4 nucleotide overhang sequence has been added to the TOPO-charged DNA and the TOPO system has been adapted to a “whole vector” format.

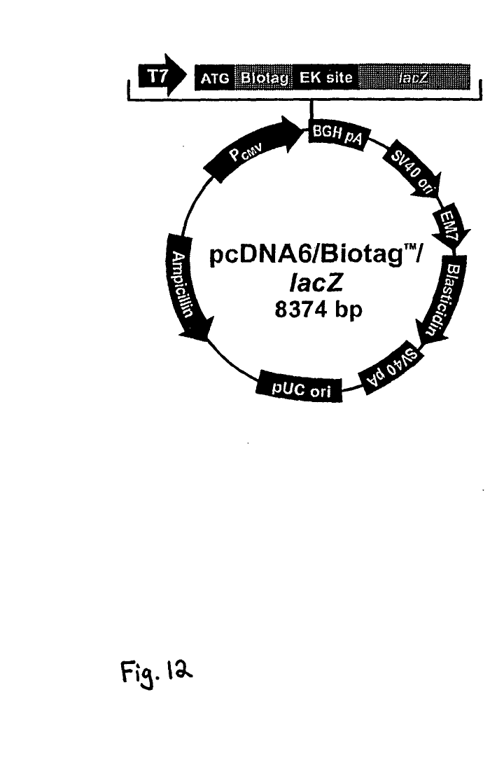

In this system, PCR products are directionally cloned by adding four bases to the forward primer (CACC). The overhang in the cloning vector (GTGG) invades the 5′ end of the PCR product, anneals to the added bases, and stabilizes the PCR product in the correct orientation (see FIG. 12). Inserts can be cloned in the correct orientation with efficiencies equal to or greater than 90%.

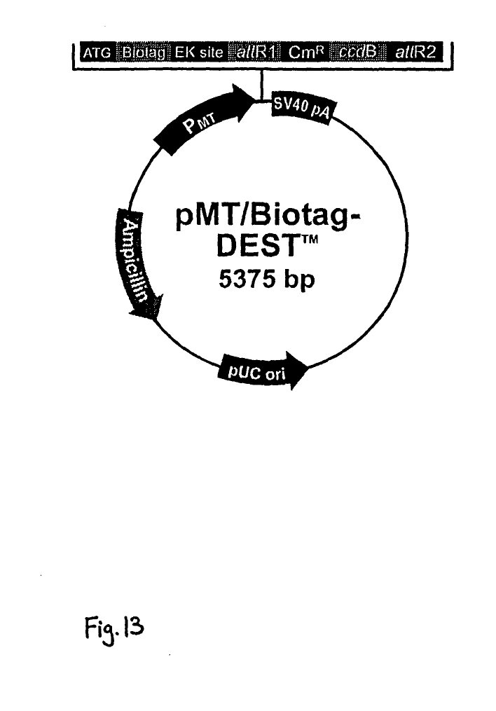

The general steps required to clone and express a blunt-end PCR product are illustrated in FIG. 13.

The following factors should be considered when designing the forward PCR primer:

(a) To enable directional cloning, the forward PCR primer must contain the sequence, CACC, at the 5′ end of the primer. The 4 nucleotides, CACC, base pair with the overhang sequence, GTGG, in the pET104/D-TOPO vector.

(b) To include the N-terminal Biotag™, it is important that the forward PCR primer be designed such that the gene of interest is in frame with the Biotag™. The initiation ATG codon is not needed. A Shine-Dalgamo ribosome binding site (RBS) is included upstream of the ATG in the N-terminal tag to ensure optimal spacing for proper translation initiation.

(c) At least six non-native amino acids will be present between the EK cleavage site and the start of the gene of interest.

(d) If it is desired to express the protein with a native N-terminus (i.e., with out the Biotag™), the forward PCR primer should be designed to include: (i) a stop codon to terminate the Biotag™, and (ii) a second ribosome binding site (AGGAGG) 9-10 base pairs 5′ of the initial ATG codon of the protein.

The following factors should be considered when designing the reverse PCR primer:

(a) It is important to include a stop codon in the reverse primer or the reverse primer should be designed to hybridize downstream of the native stop codon.

(b) To ensure that the PCR product clones directionally with high efficiency, the reverse PCR primer must not be complementary to the overhang sequence GTGG at the 5′ end. A one base pair mismatch can reduce the directional cloning efficiency from 90% to 75%, and may increase the chances of the open reading frame cloning in the opposite orientation.

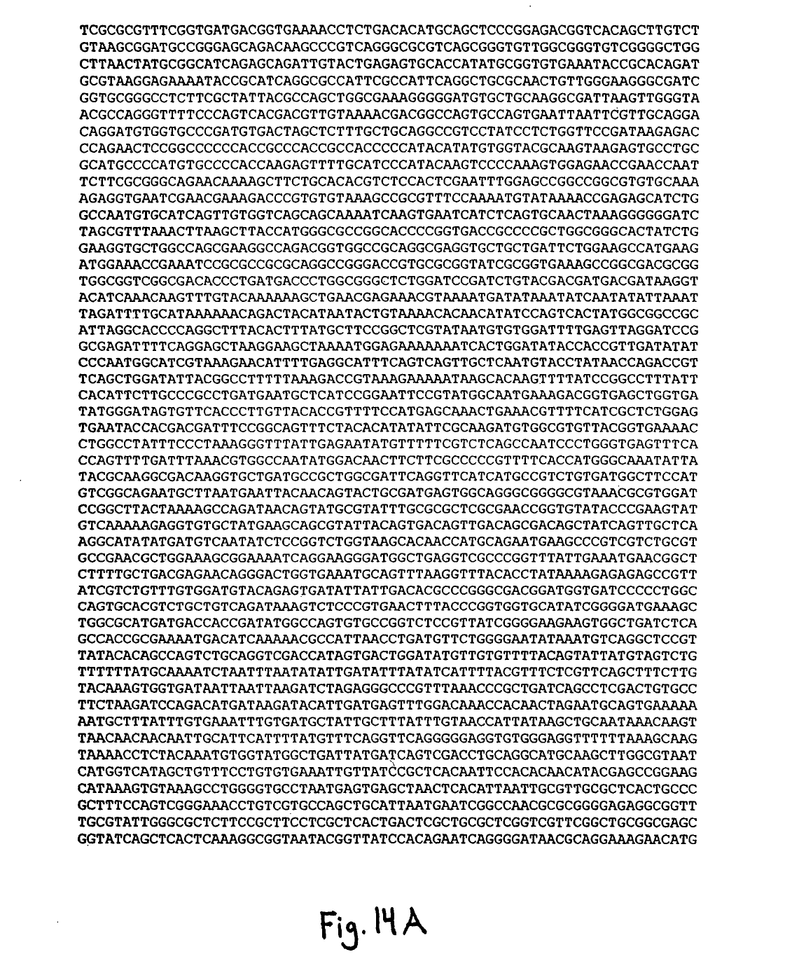

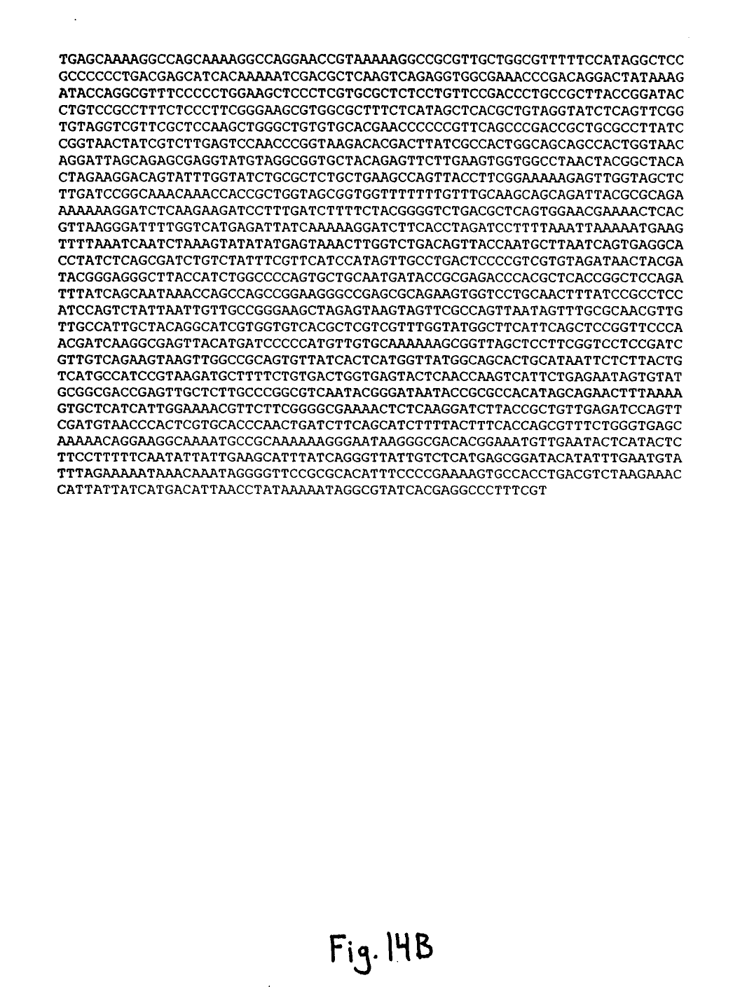

The diagram depicted in FIG. 14 is useful for designing suitable PCR primers to clone an express a PCR product using pET104/D-TOPO. The biotin binding site is designated with an asterisk (*).

Once a desired PCR product has been produced, it can then be TOPO cloned into the pET104/D-TOPO vector. The recombinant vector can then be transformed into an appropriate E. coli strain.

It has been found that inclusion of salt (e.g., 250 mM NaCl, 10 mM MgCl₂) in the TOPO cloning reaction may result in an increase in the number of transformants. Therefore, it is recommended that salt be added to the TOPO cloning reaction.

Table III describes how to set up a TOPO cloning reaction (6 μl) for eventual transformation into either chemically competent E. coli or electrocompetent E. coli.

TABLE III
Setting up a TOPO Cloning Reaction

| | Chemically competent | |
|---|---|---|
| Reagents | E. coli | Electrocompetent E. coli |
| Fresh PCR product | 0.5 to 4.0 μl | 0.5 to 4.0 μl |
| Salt solution | 1 μl | — |
| Sterile water | Add to a final volume of | Add to a final volume of |
| | 5 μl | 5 μl |
| TOPO vector | 1 μl | 1 μl |

Mix reaction gently and incubate for 5 minutes at room temperature (22-23° C.). For most applications, 5 minutes will yield sufficient colonies for analysis. Depending on the circumstances, the length of the TOPO cloning reaction can be varied from 30 seconds to 30 minutes. For routine subcloning of PCR products, 30 seconds may be sufficient. For large PCR products (>1 kb) or if a pool of PCR products is being cloned, increasing the reaction time may yield more colonies.

Place the reaction on ice or store the TOPO cloning reaction at −20° C. overnight.

Once the TOPO cloning reaction has been performed, the pET104/D-TOPO construct will be transformed into competent E. coli. Methods for transforming E. coli with nucleic acids are known in the art.

Transformants can be analyzed by isolating plasmid DNA from transformant colonies. The isolated plasmid DNA can be checked by restriction analysis to confirm the presence and correct orientation of the insert. Additionally, the construct can be sequenced to confirm that the gene of interest is in frame with the N-terminal Biotag™. Forward and T7 reverse primers can be used to sequence the insert. Positive transformants can also be analyzed by PCR.

Expression of the recombinant fusion protein can be induced by first transforming the expression clone into an appropriate E. coli strain for protein expression, e.g., BL21 cells. The transformant is then grown to mid-log in LB containing 100 μg/ml ampicillin or 50 μg/ml carbenicillin, and IPTG is added to a final concentration of 0.5-1 mM.

Expression of the recombinant fusion protein can be detected, e.g., by western blot analysis using, e.g., streptavidin-HRP or streptavidin-AP conjugates, or an antibody (or fragment thereof) specific for the protein of interest.

The recombinant fusion protein can then be purified. The presence of the N-terminal Biotag™ in pET104/D-TOPO allows the recombinant fusion protein to be biotinylated. Once biotinylated, the recombinant fusion protein can be purified by taking advantage of the strong association between biotin and avidin (and its analogs including streptavidin). For example, streptavidin agarose-conjugated beads can be used to purify the recombinant fusion protein. Other streptavidin conjugates can also be used.

A streptavidin-agarose resin can be used for affinity purification of recombinant fusion proteins containing the Biotag™. The resin can be constructed by covalently linking streptavidin to cross-linked agarose beads via a 15-atom hydrophilic spacer arm specifically designed to reduce non-specific binding and to ensure optimal binding of biotinylated molecules. Streptavidin is bound to a final concentration of 2-3 mg streptavidin per ml of packed resin.

Recombinant fusion proteins may be purified with streptavidin-agarose under native or denaturing conditions. Methods for purifying biotinylated proteins are known in the art.

pET104/D-TOPO contains an enterokinase (EK) recognition site to allow removal of the Biotag™ from the recombinant fusion protein, if desired. After digestion with enterokinase, 6 amino acids will remain at the N-terminus of the protein (see FIG. 14). Methods for digestion with enterokinase are known in the art.

### EXAMPLE 3

### A Gateway-Adapted Destination Vector for Cloning and Expression of Biotinylated Fusion Proteins in Mammalian Cells

This example describes the pcDNA/Biotag™-DEST vector (FIG. 5). pcDNA6/Biotag™-DEST is a 7.0 kb vector adapted for use with the Gateway Technology, and is designed to allow high-level expression of biotinylated recombinant fusion proteins in mammalian cells. Biotinylated recombinant protein may then be easily detected or immobilized to a solid support for other downstream applications.

The pcDNA6/Biotag™-DEST vector contains the following elements:

(a) The human cytomegalovirus (CMV) immediate early enhancer/promoter for high level constitutive expression of the gene of interest in a wide range of mammalian cells (Andersson, S. et al., J. Biol. Chem. 264:8222-8229 (1989); Boshart, M. et al., Cell 41:521-530 (1985); Nelson, J. A. et al., Molec. Cell Biol. 7:4125-4129 (1987));

(b) Biotag™ to allow biotinylation of the recombinant protein of interest for easy detection or use in other applications.

(c) Enterokinase (EK) recognition site for cleavage of the Biotag™ from the recombinant protein;

(d) Two recombination sites, attR1 and attR2, downstream of the CMV promoter for recombinational cloning of the gene of interest from an entry clone;

(e) Chloramphenicol resistance gene (CmR) located between the two attR sites for counterselection;

(f) The ccdB gene located between the attR sites for negative selection;

(g) Blasticidin (bsd) resistance gene for selection of stable cell lines using blasticidin;

(h) Ampicillin resistance gene for selection in E. coli; and

(i) pUC origin for high-copy replication and maintenance of the plasmid in E. coli.

The control plasmid, pcDNA6/Biotag™-GW/lacZ (FIG. 6), can be used as a positive control for transfection and expression in the mammalian cell line of choice. pcDNA6/Biotag™-GW/lacZ was generated using the Gateway LR recombination reaction between an entry clone containing the lacZ gene and pcDNA6/Biotag™-DEST.

To recombine a gene of interest into pcDNA6/Biotag™-DEST, an entry clone containing the gene of interest must first be obtained. Details relating to choosing an entry vector and constructing an entry clone are available in the art (See, e.g., U.S. Pat. No. 6,270,969).

pcDNA6/Biotag™-DEST is an N-terminal fusion vector and contains an ATG initiation codon in the context of a Kozak consensus sequence to ensure optimal translation initiation. The gene of interest in the entry clone must: (a) be in frame with the N-terminal Biotag™ after recombination; and (b) contain a stop codon.

The entry clone will contain, e.g., attL sites flanking the gene of interest. Genes in an entry clone are transferred to the destination vector backbone by mixing the DNAs with, e.g., the Gateway LR Clonase Enzyme Mix. The resulting LR recombination reaction is then transformed into E. coli (e.g., TOP10 or DH5α-T1R) and the expression clone is selected using ampicillin. Recombination between the attR sites on the destination vector and the attL sites on the entry clone replaces the chloramphenicol (CmR) gene and the ccdB gene with the gene of interest and results in the formation of attB sites in the expression clone. Details for setting up the recombination reaction, transforming E. coli, and selecting for the expression clone, are available in the art.

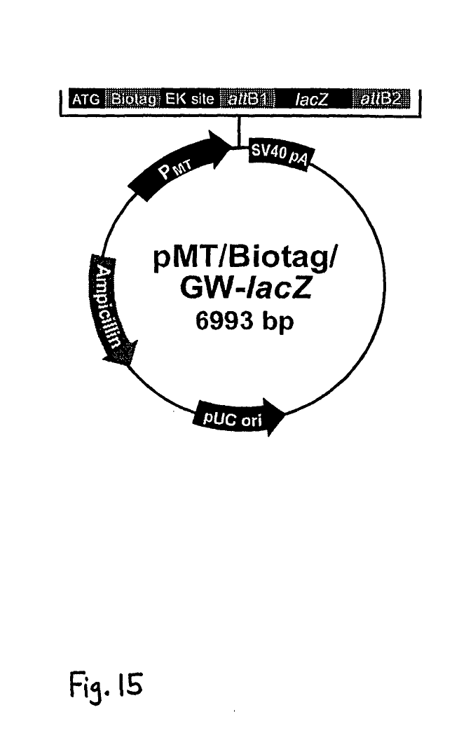

The recombination region of the expression clone resulting from pcDNA6/Biotag™-DEST x entry clone is depicted in FIG. 15. Features of the recombination region are as follows:

(a) shaded regions correspond to those DNA sequences transferred from the entry clone into the pcDNA6/Biotag™-DEST vector by recombination. Non-shaded regions are derived from the pcDNA6/Biotag™-DEST vector;

(b) bases 1191 and 2853 of the pcDNA6/Biotag™-DEST sequence are marked.

(c) The biotin binding site is labeled with an asterisk (*).

(d) Potential stop codons are underlined.

The Expression clone can be confirmed following recombination. The ccdB gene mutates at a very low frequency, resulting in a very low number of false positives. True expression clones will be ampicillin-resistant and chloramphenicol-sensitive. Transformants containing a plasmid with a mutated ccdB gene will be both ampicillin- and chloramphenicol-resistant. To check a putative expression clone, transformants can be tested for growth on LB plates containing 30 μg/ml chloramphenicol. A true expression clone should not grow in the presence of chloramphenicol.

The expression construct may also be sequenced to confirm that the gene of interest is in frame with the Biotag™. The priming sites indicated in FIG. 15 can be used to sequence the insert.

Before expression of the recombinant fusion protein can be induced, the expression clone must first be transfected into the mammalian cells of choice. Methods for transfecting mammalian cells are known in the art. Exemplary methods of transfection include calcium phosphate, lipid-mediated, and electroporation. Following transfection, a stable cell line can be generated.

Expression of the recombinant fusion protein can be assayed from either transiently transfected cells or stable cell lines. Expression of the recombinant fusion protein can be detected, e.g., by western blot analysis using, e.g., streptavidin-HRP or streptavidin-AP conjugates, or an antibody (or fragment thereof) specific for the protein of interest.

The recombinant fusion protein can then be purified. The presence of the N-terminal Biotag™ in pcDNA6/Biotag™-DEST allows the recombinant fusion protein to be biotinylated. Once biotinylated, the recombinant fusion protein can be purified by taking advantage of the strong association between biotin and avidin (and its analogs including streptavidin). For example, streptavidin agarose-conjugated beads can be used to purify the recombinant fusion protein. Other streptavidin conjugates can also be used.

A streptavidin-agarose resin can be used for affinity purification of recombinant fusion proteins containing the Biotag™. The resin can be constructed by covalently linking streptavidin to cross-linked agarose beads via a 15-atom hydrophilic spacer arm specifically designed to reduce non-specific binding and to ensure optimal binding of biotinylated molecules. Streptavidin is bound to a final concentration of 2-3 mg streptavidin per ml of packed resin.

Recombinant fusion proteins may be purified with streptavidin-agarose under native or denaturing conditions. Methods for purifying biotinylated proteins are known in the art.

pcDNA6/Biotag™-DEST contains an enterokinase (EK) recognition site to allow removal of the Biotag™ from the recombinant fusion protein, if desired. After digestion with enterokinase, 12 amino acids will remain at the N-terminus of the protein (see FIG. 15). Methods for digestion with enterokinase are known in the art.

### EXAMPLE 4

### Directional TOPO Cloning of Blunt-End PCR Products into a Vector for Biotinylated Expression in Mammalian Cells

This example describes directional TOPO cloning using the pcDNA6/Biotag™/D-TOPO vector (FIG. 7).

pcDNA6/Biotag™/D-TOPO is a 5.3 kb expression vector designed to facilitate rapid directional cloning of blunt-end PCR products for high-level expression and biotinylation in mammalian cells. Biotinylated recombinant protein may then be easily detected or immobilized to a solid support for other downstream applications. The pcDNA6/Biotag™/D-TOPO vector comprises the following elements:

(a) The human cytomegalovirus (CMV) immediate early enhancer/promoter for high level constitutive expression of the gene of interest in a wide range of mammalian cells (Andersson, S. et al., J. Biol. Chem. 264:8222-8229 (1989); Boshart, M. et al., Cell 41:521-530 (1985); Nelson, J. A. et al., Molec. Cell Biol. 7:4125-4129 (1987));

(b) Biotag™ to allow biotinylation of the recombinant protein of interest for easy detection or use in other applications;

(c) Enterokinase (EK) recognition site for cleavage of the Biotag™ from the recombinant protein;

(d) TOPO cloning site for rapid and efficient directional cloning of blunt-end PCR products;

(e) Blasticidin (bsd) resistance gene for selection of stable cell lines using blasticidin.

The control plasmid, pcDNA6/Biotag™/lacZ (FIG. 8), can be used as a positive control for expression in E. coli. The gene encoding β-galactosidase was directionally TOPO cloned into the pcDNA6/Biotag™/D-TOPO vector.

The theory behind topoisomerase cloning is described under Example 2, supra.

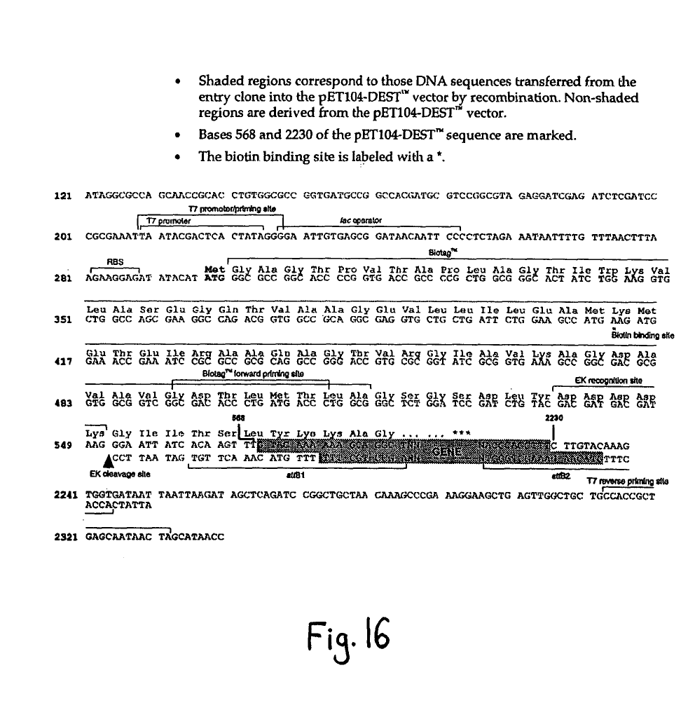

The general steps required to clone and express a blunt-end PCR product are illustrated in FIG. 16.

The following factors should be considered when designing the forward PCR primer:

(e) To enable directional cloning, the forward PCR primer must contain the sequence, CACC, at the 5′ end of the primer. The 4 nucleotides, CACC, base pair with the overhang sequence, GTGG, in the pcDNA6/Biotag™/D-TOPO vector.

(f) To include the N-terminal Biotag™, it is important that the forward PCR primer be designed such that the gene of interest is in frame with the Biotag™. The initiation ATG codon is not needed.

(g) If it is desired to express the protein with a native N-terminus (i.e., with out the Biotag™), the forward PCR primer should be designed to include: (i) a stop codon to terminate the Biotag™, and (ii) the ATG initiation codon within the context of a Kozak consensus sequence to ensure optimal translation initiation.

The following factors should be considered when designing the reverse PCR primer:

(c) It is important to include a stop codon in the reverse primer or the reverse primer should be designed to hybridize downstream of the native stop codon.

(d) To ensure that the PCR product clones directionally with high efficiency, the reverse PCR primer must not be complementary to the overhang sequence GTGG at the 5′ end. A one base pair mismatch can reduce the directional cloning efficiency from 90% to 75%, and may increase the chances of the open reading frame cloning in the opposite orientation.

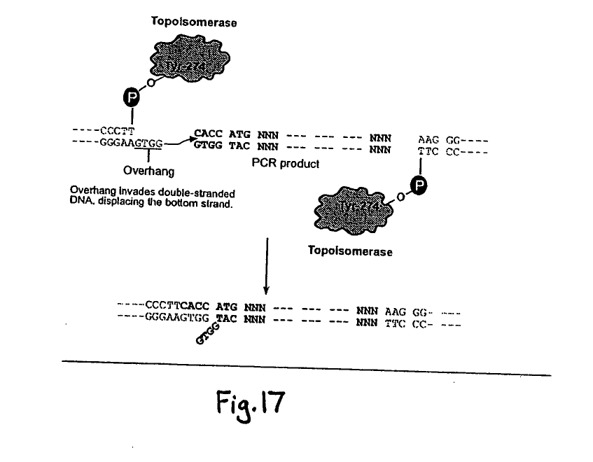

The diagram depicted in FIG. 17 is useful for designing suitable PCR primers to clone an express a PCR product using pcDNA6/Biotag™/D-TOPO. The biotin binding site is designated with an asterisk (*).

Once a desired PCR product has been produced, it can then be TOPO cloned into the pcDNA6/Biotag™/D-TOPO vector. The recombinant vector can then be transformed into an appropriate E. coli strain.

It has been found that inclusion of salt (e.g., 250 mM NaCl, 10 mM MgCl₂) in the TOPO cloning reaction may result in an increase in the number of transformants. Therefore, it is recommended that salt be added to the TOPO cloning reaction.

Table IV describes how to set up a TOPO cloning reaction (6 μl) for eventual transformation into either chemically competent E. coli or electrocompetent E. coli.

TABLE IV
Setting up a TOPO Cloning Reaction

| | Chemically competent | |
|---|---|---|
| Reagents | E. coli | Electrocompetent E. coli |
| Fresh PCR product | 0.5 to 4.0 μl | 0.5 to 4.0 μl |
| Salt solution | 1 μl | — |
| Sterile water | Add to a final volume of | Add to a final volume of |
| | 5 μl | 5 μl |
| TOPO vector | 1 μl | 1 μl |

Mix reaction gently and incubate for 5 minutes at room temperature (22-23° C.). For most applications, 5 minutes will yield sufficient colonies for analysis. Depending on the circumstances, the length of the TOPO cloning reaction can be varied from 30 seconds to 30 minutes. For routine subcloning of PCR products, 30 seconds may be sufficient. For large PCR products (>1 kb) or if a pool of PCR products is being cloned, increasing the reaction time may yield more colonies.

Place the reaction on ice or store the TOPO cloning reaction at −20° C. overnight.

Once the TOPO cloning reaction has been performed, pcDNA6/Biotag™/D-TOPO construct will be transformed into competent E. coli. Methods for transforming E. coli with nucleic acids are known in the art.

Transformants can be analyzed by isolating plasmid DNA from transformant colonies. The isolated plasmid DNA can be checked by restriction analysis to confirm the presence and correct orientation of the insert. Additionally, the construct can be sequenced to confirm that the gene of interest is in frame with the N-terminal Biotag™. Forward and T7 reverse primers can be used to sequence the insert. Positive transformants can also be analyzed by PCR.

Before expression of the recombinant fusion protein can be induced, the expression clone must first be transfected into the mammalian cells of choice. Methods for transfecting mammalian cells are known in the art. Exemplary methods of transfection include calcium phosphate, lipid-mediated, and electroporation. Following transfection, a stable cell line can be generated.

Expression of the recombinant fusion protein can be assayed from either transiently transfected cells or stable cell lines. Expression of the recombinant fusion protein can be detected, e.g., by western blot analysis using, e.g., streptavidin-HRP or streptavidin-AP conjugates, or an antibody (or fragment thereof) specific for the protein of interest.

The recombinant fusion protein can then be purified. The presence of the N-terminal Biotag™ in pcDNA6/Biotag™/D-TOPO allows the recombinant fusion protein to be biotinylated. Once biotinylated, the recombinant fusion protein can be purified by taking advantage of the strong association between biotin and avidin (and its analogs including streptavidin). For example, streptavidin agarose-conjugated beads can be used to purify the recombinant fusion protein. Other streptavidin conjugates can also be used.

A streptavidin-agarose resin can be used for affinity purification of recombinant fusion proteins containing the Biotag™. The resin can be constructed by covalently linking streptavidin to cross-linked agarose beads via a 15-atom hydrophilic spacer arm specifically designed to reduce non-specific binding and to ensure optimal binding of biotinylated molecules. Streptavidin is bound to a final concentration of 2-3 mg streptavidin per ml of packed resin.

Recombinant fusion proteins may be purified with streptavidin-agarose under native or denaturing conditions. Methods for purifying biotinylated proteins are known in the art.

pcDNA6/Biotag™/D-TOPO contains an enterokinase (EK) recognition site to allow removal of the Biotag™ from the recombinant fusion protein, if desired. After digestion with enterokinase, 13 amino acids will remain at the N-terminus of the protein (see FIG. 17). Methods for digestion with enterokinase are known in the art.

### EXAMPLE 5

### A Gateway™-Adapted Destination Vector for the Stable Expression of Biotinylated Fusion Proteins in Drosophila Schneider 2 Cells

This example describes the pMT/Biotag™-DEST vector (FIG. 9). pMT/Biotag™-DEST is a 5.4 kb vector adapted for use with the Gateway Technology, and is designed to allow high-level expression of biotinylated recombinant fusion proteins in Drosophila Schneider 2 (S2) cells. Biotinylated recombinant protein may then be easily detected or immobilized to a solid support for other downstream applications.

The pMT/Biotag™-DEST vector contains the following elements:

(a) The Drosophila metallothionein (MT) promoter for high-level, metal-inducible expression of a gene of interest in S2 cells.

(b) Biotag™ to allow biotinylation of the recombinant protein of interest for easy detection or use in other applications.

(c) Two recombination sites, attR1 and attR2, downstream of the MT promoter for recombinational cloning of the gene of interest form an entry clone.

(d) Chloramphenicol resistance gene (CmR) located between the attR sites for counterselection.

(e) The ccdb gene located between the attR sites for negative selection.

(f) pUC origin for high-copy replication and maintenance of the plasmid in E. coli.

(g) Ampicillin resistance gene for selection in E. coli.

The control plasmid, pMT/Biotag™/GW-lacZ (FIG. 10), can be used as a positive control for transfection and expression in the mammalian cell line of choice. pMT/Biotag™/GW-lacZ was generated using the Gateway LR recombination reaction between an entry clone containing the lacZ gene and pMT/Biotag™-DEST.

To recombine a gene of interest into pMT/Biotag™-DEST, an entry clone containing the gene of interest must first be obtained. Details relating to choosing an entry vector and constructing an entry clone are available in the art (See, e.g., U.S. Pat. No. 6,270,969).

pMT/Biotag™-DEST is an N-terminal fusion vector and contains an ATG initiation codon. The gene of interest in the entry clone must: (a) be in frame with the N-terminal Biotag™ after recombination; and (b) contain a stop codon.

The entry clone will contain, e.g., attL sites flanking the gene of interest. Genes in an entry clone are transferred to the destination vector backbone by mixing the DNAs with, e.g., the Gateway LR Clonase Enzyme Mix. The resulting LR recombination reaction is then transformed into E. coli (e.g., TOP10 or DH5α-T1R) and the expression clone is selected using ampicillin. Recombination between the attR sites on the destination vector and the attL sites on the entry clone replaces the chloramphenicol (CmR) gene and the ccdB gene with the gene of interest and results in the formation of attB sites in the expression clone. Details for setting up the recombination reaction, transforming E. coli, and selecting for the expression clone, are available in the art.

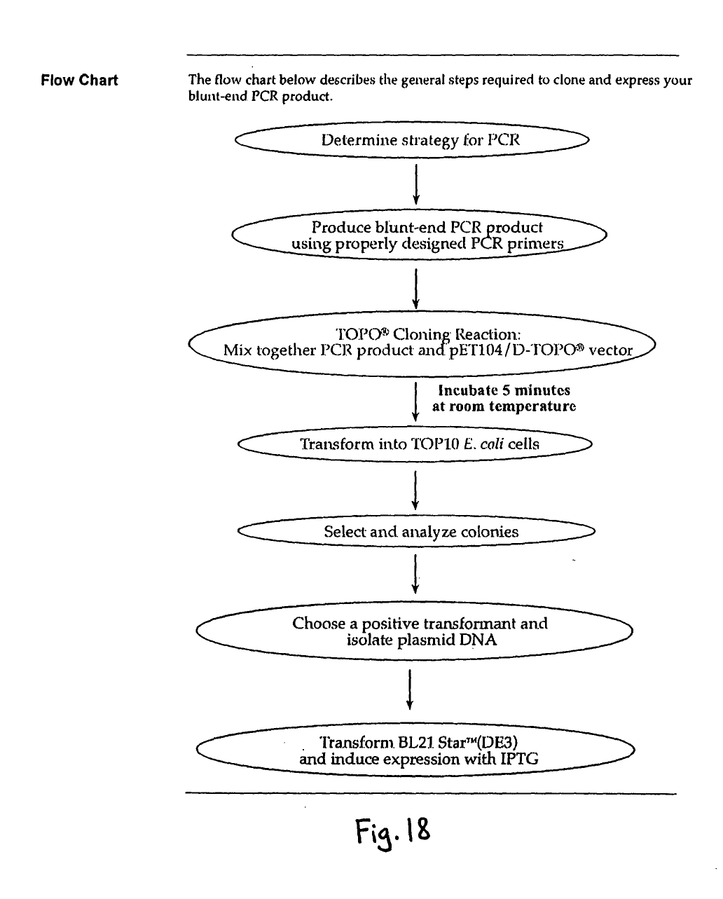

The recombination region of the expression clone resulting from pMT/Biotag™-DEST x entry clone is depicted in FIG. 18. Features of the recombination region are as follows:

(e) shaded regions correspond to those DNA sequences transferred from the entry clone into the pMT/Biotag™-DEST vector by recombination. Non-shaded regions are derived from the pMT/Biotag™-DEST vector;

(f) bases 1135 and 2797 of the pMT/Biotag™-DEST sequence are marked.

(g) The biotin binding site is labeled with an asterisk (*).

(h) Potential stop codons are underlined.

The basic steps needed to clone and express a protein using pMT/Biotag™-DEST are as follows:

(a) Establish a culture of S2 cells from supplied frozen stock.

(b) Choose a Gateway entry vector and generate an entry clone containing the gene of interest.

(c) Perform an LR recombination reaction between the entry clone containing the gene of interest and the pMT/Biotag™-DEST vector. Transform E. coli and select for the expression clone.

(d) Isolate plasmid DNA.

(e) Transiently transfect S2 cells.

(f) Induce, if necessary, and assay for expression of the protein.

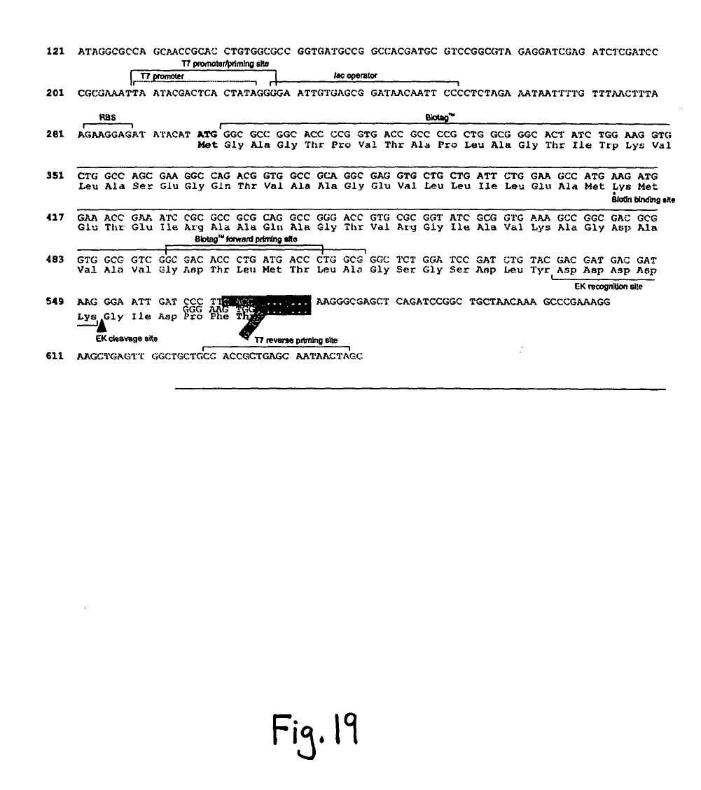

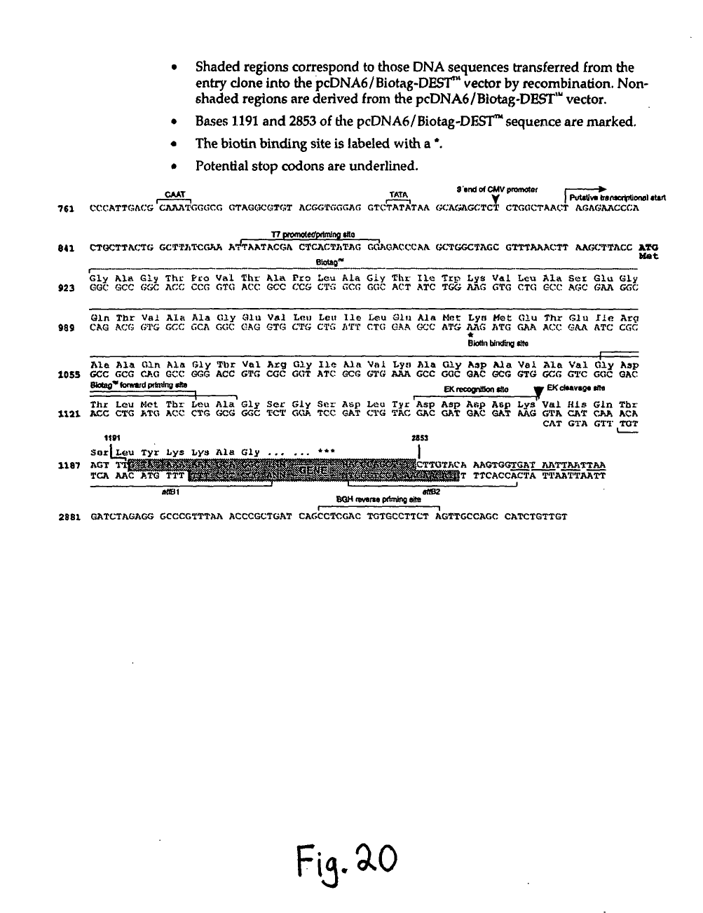

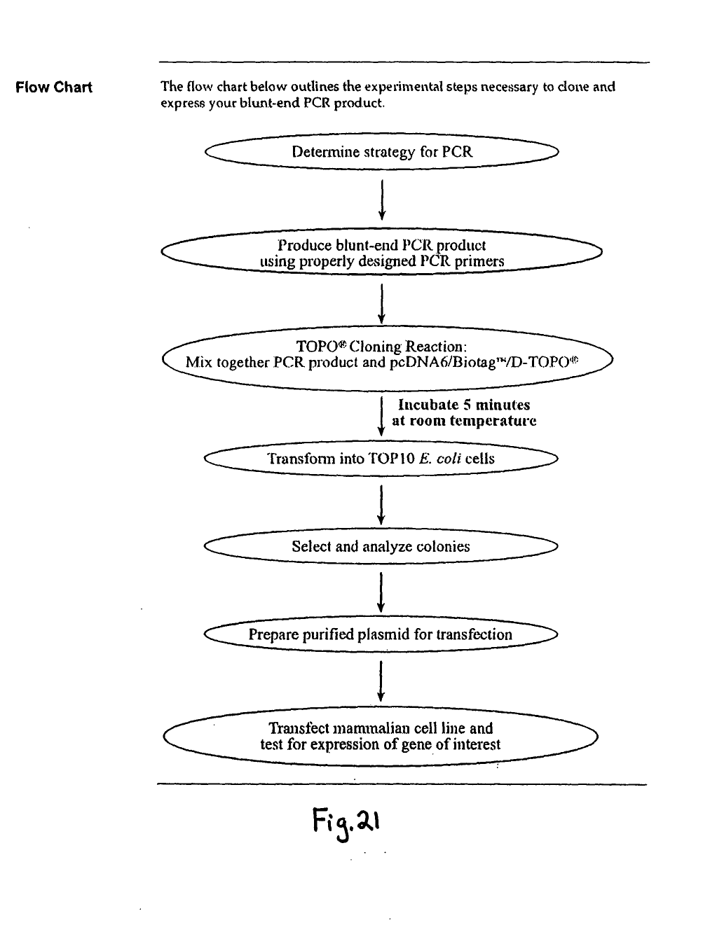

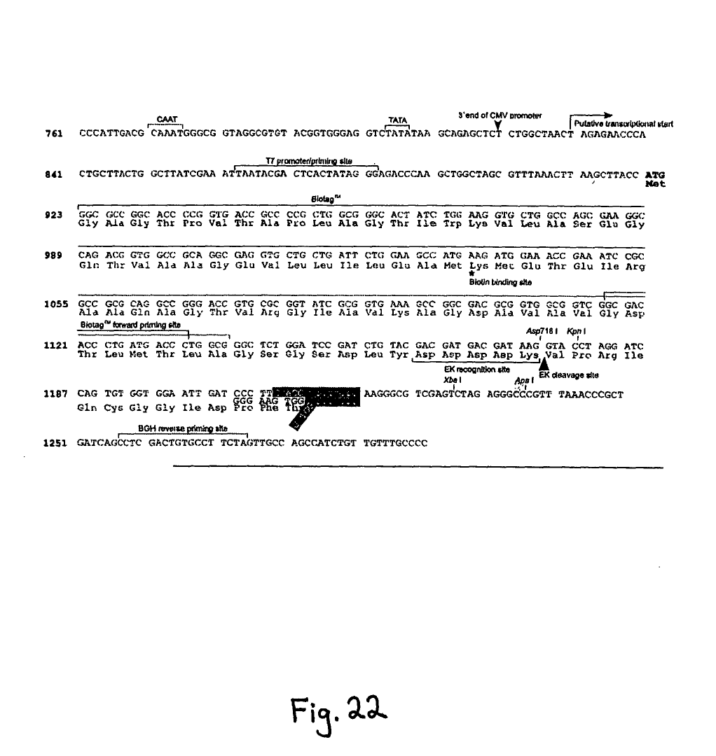

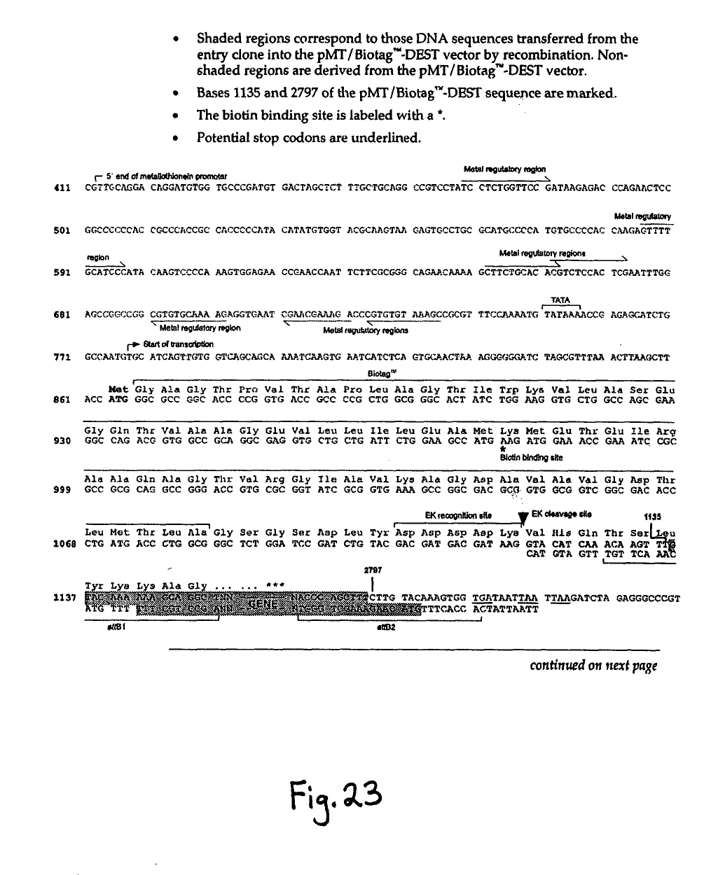

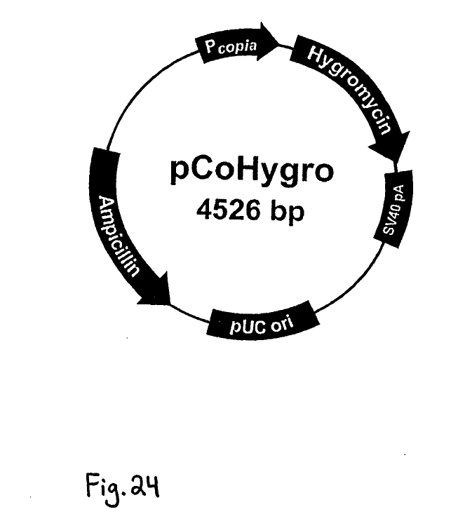

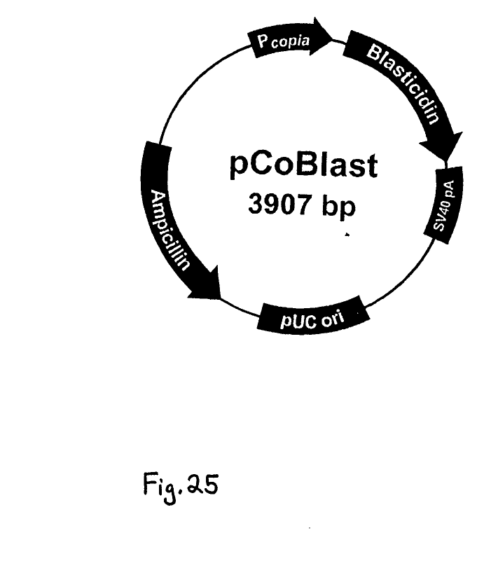

(g) Create stable cell lines expressing the protein of interest by cotransfecting the recombinant expression vector with a selection vector, pCoHygro (FIG. 19) or pCoBlast (FIG. 20), and select with the appropriate concentration of hygromycin-B or blasticidin, respectively.

(h) Induce if necessary, and assay for expression of the protein.

(i) Scale up expression, if desired.

Expression of the recombinant fusion protein can be detected, e.g., by western blot analysis using, e.g., streptavidin-HRP or streptavidin-AP conjugates, or an antibody (or fragment thereof) specific for the protein of interest.

The recombinant fusion protein can then be purified. The presence of the N-terminal Biotag™ in pMT/Biotag™-DEST allows the recombinant fusion protein to be biotinylated. Once biotinylated, the recombinant fusion protein can be purified by taking advantage of the strong association between biotin and avidin (and its analogs including streptavidin). For example, streptavidin agarose-conjugated beads can be used to purify the recombinant fusion protein. Other streptavidin conjugates can also be used.

A streptavidin-agarose resin can be used for affinity purification of recombinant fusion proteins containing the Biotag™. The resin can be constructed by covalently linking streptavidin to cross-linked agarose beads via a 15-atom hydrophilic spacer arm specifically designed to reduce non-specific binding and to ensure optimal binding of biotinylated molecules. Streptavidin is bound to a final concentration of 2-3 mg streptavidin per ml of packed resin.

Recombinant fusion proteins may be purified with streptavidin-agarose under native or denaturing conditions. Methods for purifying biotinylated proteins are known in the art.

pMT/Biotag™-DEST contains an enterokinase (EK) recognition site to allow removal of the Biotag™ from the recombinant fusion protein, if desired. After digestion with enterokinase, 11 amino acids will remain at the N-terminus of the protein (see FIG. 18). Methods for digestion with enterokinase are known in the art.

Having now fully described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

What is claimed is:

1. An isolated nucleic acid molecule comprising:
(a) one or more recombination sites; and
(b) one or more nucleic acid sequences which encode an amino acid sequence tag.

2. The isolated nucleic acid molecule of claim 1, further comprising at least one additional nucleic acid sequence selected from the group consisting of a selectable marker, a cloning site, a restriction site, a promoter, an operator, an operon, a nucleotide sequence encoding a gene product which allows for negative selection, an origin of replication, a nucleotide sequence which encodes a repressor of at least one promoter, and a gene or partial gene.

3. The isolated nucleic acid molecule of claim 1, wherein a nucleic acid sequence of interest can be inserted at or within 20 nucleotides of said one or more recombination sites, thereby producing a polynucleotide construct that encodes a fusion protein, said fusion protein comprising: (i) said amino acid sequence tag; and (ii) the amino acid sequence encoded by said nucleic acid sequence of interest.

4. The isolated nucleic acid molecule of claim 1, further comprising a nucleic acid sequence that encodes an amino acid sequence that is capable of being cleaved by one or more proteases.

5. The isolated nucleic acid molecule of claim 4, wherein said amino acid sequence that is capable of being cleaved by one or more proteases is an amino acid sequence that is capable of being cleaved by enterokinase.

6. The isolated nucleic acid molecule of claim 4, wherein a nucleic acid sequence of interest can be inserted at or within 20 nucleotides of said one or more recombination sites thereby producing a polynucleotide construct that encodes a fusion protein, said fusion protein comprising: (i) said amino acid sequence that is capable of being cleaved by one or more proteases, flanked on one side by (ii) said amino acid tag, and on the other side by (iii) the amino acid sequence encoded by said nucleic acid sequence of interest.

7. The nucleic acid molecule of claim 1, wherein said amino acid sequence tag is an amino acid sequence that is capable of being post-translationally modified.

8. The isolated nucleic acid molecule of claim 7, wherein said amino acid sequence that is capable of being post-translationally modified is an amino acid sequence that is capable of being post-translationally modified by biotinylation, attachment of 4-phosphopanthetheine, attachment of lipoic acid or attachment of flavins.

9. The isolated nucleic acid molecule of claim 7, wherein said amino acid sequence that is capable of being post-translationally modified is an amino acid sequence that is capable of being biotinylated.

10. The isolated nucleic acid molecule of claim 9, wherein said amino acid sequence that is capable of being biotinylated is all or a portion of the Klebsiella pneumoniae oxalacetate decarboxylase α subunit, all or a portion of the Propionibacterium shermanii transcarboxylase 1.3S subunit, or all or a portion of the Escherichia coli biotin carboxyl carrier protein component of acetyl-CoA carboxylase.

11. The isolated nucleic acid molecule of claim 9, wherein said amino acid sequence that is capable of being biotinylated is a portion of the C-terminus of the Klebsiella pneumoniae oxalacetate decarboxylase α subunit.

12. The isolated nucleic acid molecule of claim 11, wherein said amino acid sequence that is capable of being biotinylated is the BIOTAG™.

13. The isolated nucleic acid molecule of claim 1, wherein said nucleic acid molecule is a circular molecule.

14. The isolated nucleic acid molecule of claim 1, wherein said nucleic acid molecule comprises two or more recombination sites.

15. The isolated nucleic acid molecule of claim 1, wherein said recombination sites are selected from the group consisting of: (a) attB sites, (b) attP sites, (c) attL sites, (d) attR sites, (e) lox sites, (f) psi sites, (g) dif sites, (h) cer sites, (i) frt sites, and mutants, variants, and derivatives of the recombination sites of (a), (b), (c), (d), (e), (f), (g), (h), or (i) which retain the ability to undergo recombination.

16. A vector comprising the isolated nucleic acid molecule of claim 1.

17. A host cell comprising the isolated nucleic acid molecule of claim 1.

18. A host cell comprising the vector of claim 16.

19. A method of producing a polynucleotide construct that encodes a fusion protein that comprises an amino acid sequence tag, said method comprising:
(a) obtaining a first nucleic acid molecule comprising a nucleotide sequence of interest flanked by at least a first and at least a second recombination sites that do not recombine with each other;
(b) obtaining a second nucleic acid molecule comprising: (i) at least a third and fourth recombination sites that do not recombine with each other; and (ii) one or more nucleic acid sequences which encode an amino acid sequence tag; and
(c) contacting said first nucleic acid molecule with said second nucleic acid molecule under conditions favoring recombination between said first and third and between said second and fourth recombination sites, thereby producing a product polynucleotide construct;
wherein said product polynucleotide construct encodes a fusion protein comprising: (i) said amino acid sequence tag; and (ii) the amino acid sequence encoded by said nucleotide acid sequence of interest.

20. The method of claim 19, wherein said second nucleic acid molecule further comprises a nucleic acid sequence that encodes an amino acid sequence that is capable of being cleaved by one or more proteases; and
wherein said product polynucleotide construct encodes a fusion protein comprising: (i) said amino acid sequence that is capable of being cleaved by one or more proteases, flanked on one side by (ii) said amino acid sequence tag, and on the other side by (iii) the amino acid sequence encoded by said nucleotide sequence of interest.

21. The method of claim 20, wherein said amino acid sequence that is capable of being cleaved by one or more proteases is an amino acid sequence that is capable of being cleaved by enterokinase.

22. The method of claim 19, wherein said amino acid sequence tag is an amino acid sequence that is capable of being post-translationally modified.

23. The method of claim 22, wherein said amino acid sequence that is capable of being post-translationally modified is an amino acid sequence that is capable of being post-translationally modified by biotinylation, attachment of 4-phosphopanthetheine, attachment of lipoic acid or attachment of flavins.

24. The method of claim 22, wherein said amino acid sequence that is capable of being post-translationally modified is an amino acid sequence that is capable of being biotinylated.

25. The method of claim of claim 24, wherein said amino acid sequence that is capable of being biotinylated is all or a portion of the Klebsiella pneumoniae oxalacetate decarboxylase α subunit, all or a portion of the Propionibacterium shermanii transcarboxylase 1.3S subunit, or all or a portion of the Escherichia coli biotin carboxyl carrier protein component of acetyl-CoA carboxylase.

26. The method of claim of claim 24, wherein said amino acid sequence that is capable of being biotinylated is a portion of the C-terminus of the Klebsiella pneumoniae oxalacetate decarboxylase α subunit.

27. The method of claim 26, wherein said amino acid sequence that is capable of being biotinylated is the BIOTAG™.

28. The method of claim 19, wherein said second nucleic acid molecule is a vector.

29. The method of claim 19, wherein said first nucleic acid molecule is a circular nucleic acid molecule.

30. The method of claim 19, wherein said first nucleic acid molecule is a linear nucleic acid molecule.

31. The method of claim 30, wherein said first nucleic acid molecule is a PCR product.

32. The method of claim 19, further comprising inserting said product polynucleotide construct into a host cell.

33. The method of claim 20, further comprising inserting said product polynucleotide construct into a host cell.

34. The method of claim 19, wherein said second nucleic acid molecule comprises at least one additional nucleic acid sequence selected from the group consisting of a selectable marker, a cloning site, a restriction site, a promoter, an operator, an operon, a nucleotide sequence encoding a gene product which allows for negative selection, an origin of replication, a nucleotide sequence which encodes a repressor of at least one promoter, and a gene or partial gene.

35. The method of claim 19, wherein said first, second, third and fourth recombination sites are selected from the group consisting of: (a) attB sites, (b) attP sites, (c) attL sites, (d) attR sites, (e) lox sites, (f) psi sites, (g) dif sites, (h) cer sites, (i)frt sites, and mutants, variants, and derivatives of the recombination sites of (a), (b), (c), (d), (e), (f), (g), (h), or (i) which retain the ability to undergo recombination.

36. The method of claim 19, wherein said first and said second nucleic acid molecules are combined in the presence of at least one recombination protein.

37. The method of claim 36, wherein said recombination protein is selected from the group consisting of: (a) Cre, (b) Int, (c) IHF, (d) Xis, (e) Fis, (f) Hin, (g) Gin, (h) Cin, (i) Tn3 resolvase, (j) TndX, (k) XerC, and (l) XerD.

38. The method of claim 36, wherein said recombination protein is Cre.

39. An isolated nucleic acid molecule comprising:
(a) one or more topoisomerase recognition sites and/or one or more topoisomerases; and
(b) one or more nucleic acid sequences which encode an amino acid sequence tag.

40. The isolated nucleic acid molecule of claim 39, further comprising at least one additional nucleic acid sequence selected from the group consisting of a selectable marker, a cloning site, a restriction site, a promoter, an operator, an operon, a nucleotide sequence encoding a gene product which allows for negative selection, an origin of replication, a nucleotide sequence which encodes a repressor of at least one promoter, and a gene or partial gene.

41. The isolated nucleic acid molecule of claim 39, wherein a nucleic acid sequence of interest can be inserted at or within 20 nucleotides of said one or more topoisomerase recognition sites and/or at or within 20 nucleotide of the position of said one or more topoisomerases, thereby producing a polynucleotide construct that encodes a fusion protein, said fusion protein comprising: (i) said amino acid sequence tag; and (ii) the amino acid sequence encoded by said nucleic acid sequence of interest.

42. The isolated nucleic acid molecule of claim 39, further comprising a nucleic acid sequence that encodes an amino acid sequence that is capable of being cleaved by one or more proteases.

43. The isolated nucleic acid molecule of claim 42, wherein said amino acid sequence that is capable of being cleaved by one or more proteases is an amino acid sequence that is capable of being cleaved by enterokinase.

44. The isolated nucleic acid molecule of claim 42, wherein a nucleic acid sequence of interest can be inserted at or within 20 nucleotides of said one or more topoisomerase recognition sites and/or at the position of said one or more topoisomerases thereby producing a polynucleotide construct that encodes a fusion protein, said fusion protein comprising: (i) said amino acid sequence that is capable of being cleaved by one or more proteases, flanked on one side by (ii) said amino acid tag, and on the other side by (iii) the amino acid sequence encoded by said nucleic acid sequence of interest.

45. The isolated nucleic acid molecule of claim 39, wherein said amino acid sequence tag is an amino acid sequence that is capable of being post-translationally modified.

46. The isolated nucleic acid molecule of claim 45, wherein said amino acid sequence that is capable of being post-translationally modified is an amino acid sequence that is capable of being post-translationally modified by biotinylation, attachment of 4-phosphopanthetheine, attachment of lipoic acid or attachment of flavins.

47. The isolated nucleic acid molecule of claim 45, wherein said amino acid sequence that is capable of being post-translationally modified is an amino acid sequence that is capable of being biotinylated.

48. The isolated nucleic acid molecule of claim 47, wherein said amino acid sequence that is capable of being biotinylated is all or a portion of the Klebsiella pneumoniae oxalacetate decarboxylase α subunit, all or a portion of the Propionibacterium shermanii transcarboxylase 1.3S subunit, or all or a portion of the Escherichia coli biotin carboxyl carrier protein component of acetyl-CoA carboxylase.

49. The isolated nucleic acid molecule of claim 47, wherein said amino acid sequence that is capable of being biotinylated is a portion of the C-terminus of the Klebsiella pneumoniae oxalacetate decarboxylase α subunit.

50. The isolated nucleic acid molecule of claim 49, wherein said amino acid sequence that is capable of being biotinylated is the BIOTAG™.

51. The isolated nucleic acid molecule of claim 39, wherein said nucleic acid molecule is a circular molecule.

52. The isolated nucleic acid molecule of claim 39, wherein said nucleic acid molecule comprises two or more recombination sites.

53. The isolated nucleic acid molecule of claim 39, wherein said topoisomerase is a type I topoisomerase.

54. The isolated nucleic acid molecule of claim 53, wherein said type I topoisomerase is a type IB topoisomerase.

55. The isolated nucleic acid molecule of claim 54, wherein said type IB topoisomerase is selected from the group consisting of eukaryotic nuclear type I topoisomerase and a poxvirus topoisomerase.

56. The isolated nucleic acid molecule of claim 55, wherein said poxvirus topoisomerase is produced by or isolated from a virus selected from the group consisting of vaccinia virus, Shope fibroma virus, ORF virus, fowlpox virus, molluscum contagiosum virus and Amsacta moorei entomopoxvirus.

57. A vector comprising the isolated nucleic acid molecule of claim 39.

58. A host cell comprising the isolated nucleic acid molecule of claim 39.

59. A host cell comprising the vector of claim 57.

60. A method of producing a polynucleotide construct that encodes a fusion protein that comprises an amino acid sequence tag, said method comprising:
(a) obtaining a first nucleic acid molecule comprising a nucleotide sequence of interest;
(b) obtaining a second nucleic acid molecule comprising at least two topoisomerase recognition sites, at least one topoisomerase, and at least one nucleic acid sequence which encodes an amino acid sequence tag;
(c) mixing said first nucleic acid molecule with said second nucleic acid molecule; and
(d) incubating said mixture under conditions such that said first nucleic acid molecule is inserted into said second nucleic acid molecule between said at least two topoisomerase recognition sites, thereby producing a product polynucleotide construct;
wherein said product polynucleotide construct encodes a fusion protein comprising: (i) said amino acid sequence tag; and (ii) the amino acid sequence encoded by said nucleotide sequence of interest.

61. The method of claim 60, wherein said second nucleic acid molecule further comprises a nucleic acid sequence that encodes an amino acid sequence that is capable of being cleaved by one or more proteases; and
wherein said product polynucleotide construct encodes a fusion protein comprising: (i) said amino acid sequence that is capable of being cleaved by one or more proteases, flanked on one side by (ii) said amino acid sequence tag, and on the other side by (iii) the amino acid sequence encoded by said nucleotide sequence of interest.

62. The method of claim 61, wherein said amino acid sequence that is capable of being cleaved by one or more proteases is an amino acid sequence that is capable of being cleaved by enterokinase.

63. The method of claim 60, wherein said amino acid sequence tag is an amino acid sequence that is capable of being post-translationally modified.

64. The method of claim 63, wherein said amino acid sequence that is capable of being post-translationally modified is an amino acid sequence that is capable of being post-translationally modified by biotinylation, attachment of 4-phosphopanthetheine, attachment of lipoic acid or attachment of flavins.

65. The method of claim 63, wherein said amino acid sequence that is capable of being post-translationally modified is an amino acid sequence that is capable of being biotinylated.

66. The method of claim of claim 65, wherein said amino acid sequence that is capable of being biotinylated is all or a portion of the Klebsiella pneumoniae oxalacetate decarboxylase α subunit, all or a portion of the Propionibacterium shermanii transcarboxylase 1.3S subunit, or all or a portion of the Escherichia coli biotin carboxyl carrier protein component of acetyl-CoA carboxylase.

67. The method of claim of claim 65, wherein said amino acid sequence that is capable of being biotinylated is a portion of the C-terminus of the Klebsiella pneumoniae oxalacetate decarboxylase α subunit.

68. The method of claim 67, wherein said amino acid sequence that is capable of being biotinylated is the BIOTAG™.

69. The method of claim 60, wherein said second nucleic acid molecule is a vector.

70. The method of claim 60, wherein said first nucleic acid molecule is a linear nucleic acid molecule.

71. The method of claim 70, wherein said first nucleic acid molecule is a blunt-end nucleic acid molecule.

72. The method of claim 60, wherein said first nucleic acid molecule is a PCR product.

73. The method of claim 60, further comprising inserting said product polynucleotide construct into a host cell.

74. The method of claim 61, further comprising inserting said product polynucleotide construct into a host cell.

75. The method of claim 60, wherein said second nucleic acid molecule comprises at least one additional nucleic acid sequence selected from the group consisting of a selectable marker, a cloning site, a restriction site, a promoter, an operator, an operon, a nucleotide sequence encoding a gene product which allows for negative selection, an origin of replication, a nucleotide sequence which encodes a repressor of at least one promoter, and a gene or partial gene.

76. The method of claim 60, wherein said topoisomerase is a type I topoisomerase.

77. The method of claim 76, wherein said type I topoisomerase is a type IB topoisomerase.

78. The method of claim 77, wherein said type IB topoisomerase is selected from the group consisting of eukaryotic nuclear type I topoisomerase and a poxvirus topoisomerase.

79. The method of claim 78, wherein said poxvirus topoisomerase is produced by or isolated from a virus selected from the group consisting of vaccinia virus, Shope fibroma virus, ORF virus, fowlpox virus, molluscum contagiosum virus and Amsacta moorei entomopoxvirus.

80. An isolated nucleic acid molecule comprising:
(a) one or more recombination sites;
(b) one or more topoisomerase recognition sites and/or one or more topoisomerases; and
(c) one or more nucleic acid sequences which encode an amino acid sequence tag.

81. The isolated nucleic acid molecule of claim 80, further comprising at least one additional nucleic acid sequence selected from the group consisting of a selectable marker, a cloning site, a restriction site, a promoter, an operator, an operon, a nucleotide sequence encoding a gene product which allows for negative selection, an origin of replication, a nucleotide sequence which encodes a repressor of at least one promoter, and a gene or partial gene.

82. The isolated nucleic acid molecule of claim 80, wherein a nucleic acid sequence of interest can be inserted at or within 20 nucleotides of said one or more recombination sites, thereby producing a polynucleotide construct that encodes a fusion protein, said fusion protein comprising: (i) said amino acid sequence tag; and (ii) the amino acid sequence encoded by said nucleic acid sequence of interest.

83. The isolated nucleic acid molecule of claim 80, wherein a nucleic acid sequence of interest can be inserted at or within 20 nucleotides of said one or more topoisomerase recognition sites and/or at or within 20 nucleotides of the position of said one or more topoisomerases, thereby producing a polynucleotide construct that encodes a fusion protein, said fusion protein comprising: (i) said amino acid tag; and (ii) the amino acid sequence encoded by said nucleic acid sequence of interest.

84. The isolated nucleic acid molecule of claim 80, further comprising a nucleic acid sequence that encodes an amino acid sequence that is capable of being cleaved by one or more proteases.

85. The isolated nucleic acid molecule of claim 84, wherein said amino acid sequence that is capable of being cleaved by one or more proteases is an amino acid sequence that is capable of being cleaved by enterokinase.

86. The isolated nucleic acid molecule of claim 84, wherein a nucleic acid sequence of interest can be inserted at or within 20 nucleotides of said one or more recombination sites, thereby producing a polynucleotide construct that encodes a fusion protein, said fusion protein comprising: (i) said amino acid sequence that is capable of being cleaved by one or more proteases, flanked on one side by (ii) said amino acid sequence tag, and on the other side by (iii) the amino acid sequence encoded by said nucleic acid sequence of interest.

87. The isolated nucleic acid molecule of claim 84, wherein a nucleic acid sequence of interest can be inserted at or within 20 nucleotides of said one or more topoisomerase recognition sites and/or at or within 20 nucleotides of the position of said one or more topoisomerases, thereby producing a polynucleotide construct that encodes a fusion protein, said fusion protein comprising: (i) said amino acid sequence that is capable of being cleaved by one or more proteases, flanked on one side by (ii) said amino acid sequence tag, and on the other side by (iii) the amino acid sequence encoded by said nucleic acid sequence of interest.

88. The isolated nucleic acid molecule of claim 80, wherein said amino acid sequence tag is an amino acid sequence that is capable of being post-translationally modified.

89. The isolated nucleic acid molecule of claim 88, wherein said amino acid sequence that is capable of being post-translationally modified is an amino acid sequence that is capable of being post-translationally modified by biotinylation, attachment of 4-phosphopanthetheine, attachment of lipoic acid or attachment of flavins.

90. The isolated nucleic acid molecule of claim 80, wherein said amino acid sequence that is capable of being post-translationally modified is an amino acid sequence that is capable of being biotinylated.

91. The isolated nucleic acid molecule of claim 90, wherein said amino acid sequence that is capable of being biotinylated is all or a portion of the Klebsiella pneumoniae oxalacetate decarboxylase α subunit, all or a portion of the Propionibacterium shermanii transcarboxylase 1.3S subunit, or all or a portion of the Escherichia coli biotin carboxyl carrier protein component of acetyl-CoA carboxylase.

92. The isolated nucleic acid molecule of claim 90, wherein said amino acid sequence that is capable of being biotinylated is a portion of the C-terminus of the Klebsiella pneumoniae oxalacetate decarboxylase α subunit.

93. The isolated nucleic acid molecule of claim 92, wherein said amino acid sequence that is capable of being biotinylated is the BIOTAG™.

94. The isolated nucleic acid molecule of claim 80, wherein said nucleic acid molecule is a circular molecule.

95. The isolated nucleic acid molecule of claim 80, wherein said nucleic acid molecule comprises two or more recombination sites.

96. The isolated nucleic acid molecule of claim 80, wherein said recombination sites are selected from the group consisting of: (a) attB sites, (b) attP sites, (c) attL sites, (d) attR sites, (e) lox sites, (f) psi sites, (g) dif sites, (h) cer sites, (i) frt sites, and mutants, variants, and derivatives of the recombination sites of (a), (b), (c), (d), (e), (f), (g), (h), or (i) which retain the ability to undergo recombination.

97. The isolated nucleic acid molecule of claim 80, wherein said topoisomerase is a type I topoisomerase.

98. The isolated nucleic acid molecule of claim 97, wherein said type I topoisomerase is a type IB topoisomerase.

99. The isolated nucleic acid molecule of claim 98, wherein said type IB topoisomerase is selected from the group consisting of eukaryotic nuclear type I topoisomerase and a poxvirus topoisomerase.

100. The isolated nucleic acid molecule of claim 99, wherein said poxvirus topoisomerase is produced by or isolated from a virus selected from the group consisting of vaccinia virus, Shope fibroma virus, ORF virus, fowlpox virus, molluscum contagiosum virus and Amsacta moorei entomopoxvirus.

101. A vector comprising the isolated nucleic acid molecule of claim 80.

102. A host cell comprising the isolated nucleic acid molecule of claim 80.

103. A host cell comprising the vector of claim 101.

104. A method of producing a polynucleotide construct that encodes a fusion protein that comprises an amino acid sequence tag, said method comprising:
(a) obtaining a first nucleic acid molecule comprising a nucleotide sequence of interest;
(b) obtaining a second nucleic acid molecule comprising (i) at least a first topoisomerase recognition site flanked by (ii) at least a first recombination site, and (iii) at least a second topoisomerase recognition site flanked by (iv) at least a second recombination site, wherein said first and second recombination sites do not recombine with each other, and (v) at least one topoisomerase;
(c) obtaining a third nucleic acid molecule comprising: (i) at least a third and fourth recombination sites that do not recombine with each other; and (ii) one or more nucleic acid sequences which encode an amino acid sequence tag;
(d) mixing said first nucleic acid molecule with said second nucleic acid molecule;
(e) incubating said mixture under conditions such that said first nucleic acid molecule is inserted into said second nucleic acid molecule between said at least two topoisomerase recognition sites, thereby producing a first product polynucleotide construct;
(f) contacting said first product polynucleotide construct with said third nucleic acid molecule under conditions favoring recombination between said first and third and between said second and fourth recombination sites, thereby producing a second product polynucleotide construct;
wherein said second product polynucleotide construct encodes a fusion protein comprising: (i) said amino acid sequence tag; and (ii) the amino acid sequence encoded by said nucleotide sequence of interest.

105. The method of claim 104, wherein said third nucleic acid molecule further comprises a nucleic acid sequence that encodes an amino acid sequence that is capable of being cleaved by one or more proteases; and
wherein said second product polynucleotide construct encodes a fusion protein comprising: (i) said amino acid sequence that is capable of being cleaved by one or more proteases, flanked on one side by (ii) said amino acid sequence tag, and on the other side by (iii)the amino acid sequence encoded by said nucleotide sequence of interest.

106. The method of claim 105, wherein said amino acid sequence that is capable of being cleaved by one or more proteases is an amino acid sequence that is capable of being cleaved by enterokinase.

107. The method of claim 104, wherein said amino acid sequence tag is an amino acid sequence that is capable of being post-translationally modified.

108. The method of claim 107, wherein said amino acid sequence that is capable of being post-translationally modified is an amino acid sequence that is capable of being post-translationally modified by biotinylation, attachment of 4-phosphopanthetheine, attachment of lipoic acid or attachment of flavins.

109. The method of claim 107, wherein said amino acid sequence that is capable of being post-translationally modified is an amino acid sequence that is capable of being biotinylated.

110. The method of claim of claim 109, wherein said amino acid sequence that is capable of being biotinylated is all or a portion of the Klebsiella pneumoniae oxalacetate decarboxylase α subunit, all or a portion of the Propionibacterium shermanii transcarboxylase 1.3S subunit, or all or a portion of the Escherichia coli biotin carboxyl carrier protein component of acetyl-CoA carboxylase.

111. The method of claim of claim 109, wherein said amino acid sequence that is capable of being biotinylated is a portion of the C-terminus of the Klebsiella pneumoniae oxalacetate decarboxylase α subunit.

112. The method of claim 111, wherein said amino acid sequence that is capable of being biotinylated is the BIOTAG™.

113. The method of claim 104, wherein said second nucleic acid molecule is a vector.

114. The method of claim 104, wherein said third nucleic acid molecule is a vector.

115. The method of claim 104, wherein said first nucleic acid molecule is a linear nucleic acid molecule.

116. The method of claim 115, wherein said first nucleic acid molecule is a blunt-end nucleic acid molecule.

117. The method of claim 104, wherein said first nucleic acid molecule is a PCR product.

118. The method of claim 104, further comprising inserting said first product polynucleotide construct into a host cell.

119. The method of claim 104, further comprising inserting said second product polynucleotide construct into a host cell.

120. The method of claim 104, wherein said second and/or said third nucleic acid molecules comprises at least one additional nucleic acid sequence selected from the group consisting of a selectable marker, a cloning site, a restriction site, a promoter, an operator, an operon, a nucleotide sequence encoding a gene product which allows for negative selection, an origin of replication, a nucleotide sequence which encodes a repressor of at least one promoter, and a gene or partial gene.

121. The method of claim 104, wherein said first, second, third and fourth recombination sites are selected from the group consisting of: (a) attB sites, (b) attP sites, (c) attL sites, (d) attR sites, (e) lox sites, (f) psi sites, (g) dif sites, (h) cer sites, (i) frt sites, and mutants, variants, and derivatives of the recombination sites of (a), (b), (c), (d), (e), (f), (g), (h), or (i) which retain the ability to undergo recombination.

122. The method of claim 104, wherein said topoisomerase is a type I topoisomerase.

123. The method of claim 122, wherein said type I topoisomerase is a type IB topoisomerase.

124. The method of claim 123, wherein said type IB topoisomerase is selected from the group consisting of eukaryotic nuclear type I topoisomerase and a poxvirus topoisomerase.

125. The method of claim 124, wherein said poxvirus topoisomerase is produced by or isolated from a virus selected from the group consisting of vaccinia virus, Shope fibroma virus, ORF virus, fowlpox virus, molluscum contagiosum virus and Amsacta moorei entomopoxvirus.

126. The method of claim 104, wherein said first product polynucleotide construct and said third nucleic acid molecule are combined in the presence of at least one recombination protein.

127. The method of claim 126, wherein said recombination protein is selected from the group consisting of: (a) Cre, (b) Int, (c) IHF, (d) Xis, (e) Fis, (f) Hin, (g) Gin, (h) Cin, (i) Tn3 resolvase, (j) TndX, (k) XerC, and (l) XerD.

128. The method of claim 126, wherein said recombination protein is Cre.

129. A vector selected from the group consisting of pET104-DEST, pET 104/GW/lacZ, pET 104/D-TOPO, pET 104/D/lacZ, pcDNA6/Biotag™-DEST, pcDNA6/Biotag™-GW/lacZ, pcDNA6/Biotag™/D-TOPO, pcDNA6/Biotag™/lacZ, pMT/Biotag™-DEST, and pMT/Biotag™/GW-lacZ.

130. A kit comprising the isolated nucleic acid molecule of claim 1.

131. The kit of claim 130, further comprising one or more components selected from the group consisting of one or more topoisomerases, one or more recombination proteins, one or more vectors, one or more polypeptides having polymerase activity, one or more host cells, and one or more support matrices complexed with avidin or an avidin analog.

132. A kit comprising the isolated nucleic acid molecule of claim 39.

133. The kit of claim 132, further comprising one or more components selected from the group consisting of one or more topoisomerases, one or more recombination proteins, one or more vectors, one or more polypeptides having polymerase activity, one or more host cells, and one or more support matrices complexed with avidin or an avidin analog.

134. A kit comprising the isolated nucleic acid molecule of claim 80.

135. The kit of claim 134, further comprising one or more components selected from the group consisting of one or more topoisomerases, one or more recombination proteins, one or more vectors, one or more polypeptides having polymerase activity, one or more host cells, and one or more support matrices complexed with avidin or an avidin analog.

136. A host cell comprising a polynucleotide construct that encodes a fusion protein capable of being post-translationally modified, said polynucleotide construct produced according to the method of claim 19.

137. A host cell comprising a polynucleotide construct that encodes a fusion protein capable of being post-translationally modified, said polynucleotide construct produced according to the method of claim 60.

138. A host cell comprising a polynucleotide construct that encodes a fusion protein capable of being post-translationally modified, said polynucleotide construct produced according to the method of claim 104.

139. A method of producing a fusion protein that comprises an amino acid sequence tag, said method comprising:
(a) obtaining the host cell of claim 136; and
(b) culturing said host cell under conditions wherein said fusion protein is produced by said host cell.

140. The method of claim 139, wherein said amino acid sequence tag is an amino acid sequence that is capable of being post-translationally modified.

141. The method of claim 140, further comprising culturing said host cell under conditions wherein said fusion protein is post-translationally modified in said host cell.

142. The method of claim 140, further comprising culturing said host cell under conditions wherein said fusion protein is biotinylated in said host cell.

143. The method of claim 139, further comprising:
(a) treating said host cell such that said fusion protein is released from said host cell; and
(b) contacting said fusion protein with a detecting composition comprising a molecule that is capable of interacting with said amino acid sequence tag or with a molecular entity that is attached to said amino acid sequence tag.

144. The method of claim 143, wherein said fusion protein is a biotinylated fusion protein, and said detecting composition comprises avidin or an avidin analogue.

145. A method of producing a fusion protein that comprises an amino acid sequence tag, said method comprising:
(a) obtaining the host cell of claim 137; and
(b) culturing said host cell under conditions wherein said fusion protein is produced by said host cell.

146. The method of claim 145, wherein said amino acid sequence tag is an amino acid sequence that is capable of being post-translationally modified.

147. The method of claim 146, further comprising culturing said host cell under conditions wherein said fusion protein is post-translationally modified in said host cell.

148. The method of claim 146, further comprising culturing said host cell under conditions wherein said fusion protein is biotinylated in said host cell.

149. The method of claim 145, further comprising:
(a) treating said host cell such that said fusion protein is released from said host cell; and
(b) contacting said fusion protein with a detecting composition comprising a molecule that is capable of interacting with said amino acid sequence tag or with a molecular entity that is attached to said amino acid sequence tag.

150. The method of claim 149, wherein said fusion protein is a biotinylated fusion protein, and said detecting composition comprises avidin or an avidin analogue.

151. A method of producing a fusion protein that comprises an amino acid sequence tag, said method comprising:
(a) obtaining the host cell of claim 138; and
(b) culturing said host cell under conditions wherein said fusion protein is produced by said host cell.

152. The method of claim 151, wherein said amino acid sequence tag is an amino acid sequence that is capable of being post-translationally modified.

153. The method of claim 152, further comprising culturing said host cell under conditions wherein said fusion protein is post-translationally modified in said host cell.

154. The method of claim 152, further comprising culturing said host cell under conditions wherein said fusion protein is biotinylated in said host cell.

155. The method of claim 151, further comprising:
(a) treating said host cell such that said fusion protein is released from said host cell; and
(b) contacting said fusion protein with a detecting composition comprising a molecule that is capable of interacting with said amino acid sequence tag or with a molecular entity that is attached to said amino acid sequence tag.

156. The method of claim 155, wherein said post-translationally modified fusion protein is a biotinylated fusion protein, and said detecting composition comprises avidin or an avidin analogue.


SEQUENCE LISTING (34 sequences)

SEQ ID NO: 1 - DNA, 7618 bp, Artificial
  pET104-DEST
caaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgggcgccggcaccccggtgaccgccccgctggcgggcactatctggaaggtgctggccagcgaaggccagacggtggccgcaggcgaggtgctgctgattctggaagccatgaagatggaaaccgaaatccgcgccgcgcaggccgggaccgtgcgcggtatcgcggtgaaagccggcgacgcggtggcggtcggcgacaccctgatgaccctggcgggctctggatccgatctgtacgacgatgacgataagggaattatcacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaacgcgtggatccggcttactaaaagccagataacagtatgcgtatttgcgcgcaccggtgctagcgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctccgttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgataattaattaagatagctcagatccggctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagctagcttatcgatgataagctgtcaaacatgagaattaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatg

SEQ ID NO: 2 - DNA, 5934 bp, Artificial
  pET104/D-TOPO
caaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgggcgccggcaccccggtgaccgccccgctggcgggcactatctggaaggtgctggccagcgaaggccagacggtggccgcaggcgaggtgctgctgattctggaagccatgaagatggaaaccgaaatccgcgccgcgcaggccgggaccgtgcgcggtatcgcggtgaaagccggcgacgcggtggcggtcggcgacaccctgatgaccctggcgggctctggatccgatctgtacgacgatgacgataagggaattgatcccttcaccaagggcgagctcagatccggctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagctagcttatcgatgataagctgtcaaacatgagaattaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatg

SEQ ID NO: 3 - DNA, 6959 bp, Artificial
  pcDNA/Biotag-DEST
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttaccatgggcgccggcaccccggtgaccgccccgctggcgggcactatctggaaggtgctggccagcgaaggccagacggtggccgcaggcgaggtgctgctgattctggaagccatgaagatggaaaccgaaatccgcgccgcgcaggccgggaccgtgcgcggtatcgcggtgaaagccggcgacgcggtggcggtcggcgacaccctgatgaccctggcgggctctggatccgatctgtacgacgatgacgataaggtacatcaaacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaacgcgtggatccggcttactaaaagccagataacagtatgcgtatttgcgcgctcgcgaaccggtgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctccgttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgataattaattaagatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 4 - DNA, 5302 bp, Artificial
  pcDNA6/Biotag/D-TOPO
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttaccatgggcgccggcaccccggtgaccgccccgctggcgggcactatctggaaggtgctggccagcgaaggccagacggtggccgcaggcgaggtgctgctgattctggaagccatgaagatggaaaccgaaatccgcgccgcgcaggccgggaccgtgcgcggtatcgcggtgaaagccggcgacgcggtggcggtcggcgacaccctgatgaccctggcgggctctggatccgatctgtacgacgatgacgataaggtacctaggatccagtgtggtggaattgatcccttcaccaagggcgtcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 5 - DNA, 5375 bp, Artificial
  pMT/Biotag-DEST
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccagtgaattaattcgttgcaggacaggatgtggtgcccgatgtgactagctctttgctgcaggccgtcctatcctctggttccgataagagacccagaactccggccccccaccgcccaccgccacccccatacatatgtggtacgcaagtaagagtgcctgcgcatgccccatgtgccccaccaagagttttgcatcccatacaagtccccaaagtggagaaccgaaccaattcttcgcgggcagaacaaaagcttctgcacacgtctccactcgaatttggagccggccggcgtgtgcaaaagaggtgaatcgaacgaaagacccgtgtgtaaagccgcgtttccaaaatgtataaaaccgagagcatctggccaatgtgcatcagttgtggtcagcagcaaaatcaagtgaatcatctcagtgcaactaaaggggggatctagcgtttaaacttaagcttaccatgggcgccggcaccccggtgaccgccccgctggcgggcactatctggaaggtgctggccagcgaaggccagacggtggccgcaggcgaggtgctgctgattctggaagccatgaagatggaaaccgaaatccgcgccgcgcaggccgggaccgtgcgcggtatcgcggtgaaagccggcgacgcggtggcggtcggcgacaccctgatgaccctggcgggctctggatccgatctgtacgacgatgacgataaggtacatcaaacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaacgcgtggatccggcttactaaaagccagataacagtatgcgtatttgcgcgctcgcgaaccggtgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctccgttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgataattaattaagatctagagggcccgtttaaacccgctgatcagcctcgactgtgccttctaagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgt

SEQ ID NO: 6 - protein, 72 aa, Klebsiella pneumoniae
GAGTPVTAPLAGTIWKVLASEGQTVAAGEVLLILEAMKMETEIRAAQAGTVRGIAVKAGDAVAVGDTLMTLA

SEQ ID NO: 7 - protein, 115 aa, Mus musculus
KALAVSDLNRAGQRQVFFELNGQLRSILVKDTQAMKEMHFHPKALKDVKGQIGAPMPGKVIDIKVAAGDKVAKGQPLCVLSAMKMETVVTSPMEGTIRKVHVTKDMTLEGDDLIL

SEQ ID NO: 8 - protein, 123 aa, Propionibacterium shermanii
MKLKVTVNGTAYDVDVDVDKSHENPMGTILFGGGTGGAPAPRAAGGAGAGKAGEGEIPAPLAGTVSKILVKEGDTVKAGQTVLVLEAMKMETEINAPTDGKVEKVLVKERDAVQGGQGLIKIG

SEQ ID NO: 9 - protein, 122 aa, Homo sapiens
GSCVEVDVHRLSDGGLLLSYDGSSYTTYMKEEVDRYRITIGNKTCVFEKENDPSVMRSPSAGKLIQYIVEDGGHVFAGQCYAEIEVMKMVMTLTAVESGCIHYVKRPGAALDPGCVLAKMQL

SEQ ID NO: 10 - protein, 156 aa, Escherichia coli
MDIRKIKKLIELVEESGISELEISEGEESVRISRAAPAASFPVMQQAYAAPMMQQPAQSNAAAPATVPSMEAPAAAEISGHIVRSPMVGTFYRTPSPDAKAFIEVGQKVNVGDTLCIVEAMKMMNQIEADKSGTVKAILVESGQPVEFDEPLVVIE

SEQ ID NO: 11 - DNA, 216 bp, Klebsiella pneumoniae
ggcgccggcaccccggtgaccgccccgctggcgggcactatctggaaggtgctggccagcgaaggccagacggtggccgcaggcgaggtgctgctgattctggaagccatgaagatggaaaccgaaatccgcgccgcgcaggccgggaccgtgcgcggtatcgcggtgaaagccggcgacgcggtggcggtcggcgacaccctgatgaccctggcg

SEQ ID NO: 12 - DNA, 345 bp, Mus musculus
aaagccctggctgtaagcgacctgaaccgtgctggccagaggcaggtgttctttgaactcaatgggcagcttcgatccattctggttaaagacacccaggccatgaaggagatgcacttccatcccaaggctttgaaggatgtgaagggccaaattggggccccgatgcctgggaaggtcatagacatcaaggtggcagcaggggacaaggtggctaagggccagcccctctgtgtgctcagcgccatgaagatggagactgtggtgacttcgcccatggagggcactatccgaaaggttcatgttaccaaggacatgactctggaaggcgacgacctcatccta

SEQ ID NO: 13 - DNA, 369 bp, Propionibacterium shermanii
atgaaactgaaggtaacagtcaacggcactgcgtatgacgttgacgttgacgtcgacaagtcacacgaaaacccgatgggcaccatcctgttcggcggcggcaccggcggcgcgccggcaccgcgcgcagcaggtggcgcaggcgccggtaaggccggagagggcgagattcccgctccgctggccggcaccgtctccaagatcctcgtgaaggagggtgacacggtcaaggctggtcagaccgtgctcgttctcgaggccatgaagatggagaccgagatcaacgctcccaccgacggcaaggtcgagaaggtccttgtcaaggagcgtgacgccgtgcagggcggtcagggtctcatcaagatcggc

SEQ ID NO: 14 - DNA, 366 bp, Homo sapiens
ggctcatgtgtagaagtagatgtacatcggctgagtgacggtggactgctcttgtcctatgatggcagcagttacaccacgtatatgaaggaggaagtagacagatatcgcatcacaattggcaataaaacctgtgtgtttgagaaggaaaatgacccatcggtgatgcgctcaccttctgctgggaagttaatccagtacattgtagaagatggaggtcatgtgtttgccggccagtgctatgcagagattgaggtaatgaagatggtaatgactttgacagctgtggagtctggctgtatccattacgtcaagcgtcctggagcagctcttgaccctggctgtgtactcgccaaaatgcaactg

SEQ ID NO: 15 - DNA, 468 bp, Escherichia coli
atggatattcgtaagattaaaaaactgatcgagctggttgaagaatcaggcatctccgaactggaaatttctgaaggcgaagagtcagtacgcattagccgtgcagctcctgccgcaagtttccctgtgatgcaacaagcttacgctgcaccaatgatgcagcagccagctcaatctaacgcagccgctccggcgaccgttccttccatggaagcgccagcagcagcggaaatcagtggtcacatcgtacgttccccgatggttggtactttctaccgcaccccaagcccggacgcaaaagcgttcatcgaagtgggtcagaaagtcaacgtgggcgataccctgtgcatcgttgaagccatgaaaatgatgaaccagatcgaagcggacaaatccggtaccgtgaaagcaattctggtcgaaagtggacaaccggtagaatttgacgagccgctggtcgtcatcgag

SEQ ID NO: 16 - protein, 8 aa, Artificial
  FLAG epitope
DYKDDDDK

SEQ ID NO: 17 - protein, 8 aa, Artificial
  FLAG epitope
DYKDEDDK

SEQ ID NO: 18 - protein, 9 aa, Artificial
  Strep epitope
AWRHPQFGG

SEQ ID NO: 19 - protein, 11 aa, Artificial
  VSV-G epitope
YTDIEMNRLGK

SEQ ID NO: 20 - protein, 6 aa, Artificial
  poly-His epitope
HHHHHH

SEQ ID NO: 21 - protein, 13 aa, Artificial
  Influenza epitope
YPYDVPDYAIEGR

SEQ ID NO: 22 - protein, 11 aa, Artificial
  Human c-myc epitope
EQKLLSEEDLN

SEQ ID NO: 23 - protein, 3 aa, Artificial
  tripeptide epitope
EEF

SEQ ID NO: 24 - protein, 5 aa, Artificial
  enterokinase (EK) recognition site
DDDDK

SEQ ID NO: 25 - DNA, 467 bp, Artificial
  pET104-DEST vector
ataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgggcgccggcaccccggtgaccgccccgctggcgggcactatctggaaggtgctggccagcgaaggccagacggtggccgcaggcgaggtgctgctgattctggaagccatgaagatggaaaccgaaatccgcgccgcgcaggccgggaccgtgcgcggtatcgcggtgaaagccggcgacgcggtggcggtcggcgacaccctgatgaccctggcgggctctggatccgatctgtacgacgatgacgataagggaattatcacaagtttgtacaaaaaagcaggctnn

SEQ ID NO: 26 - protein, 96 aa, Artificial
  pET104-DEST vector
MGAGTPVTAPLAGTIWKVLASEGQTVAAGEVLLILEAMKMETEIRAAQAGTVRGIAVKAGDAVAVGDTLMTLAGSGSDLYDDDDKGIITSLYKKAG

SEQ ID NO: 27 - DNA, 449 bp, Artificial
  pET104/D-TOPO vector
ataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgggcgccggcaccccggtgaccgccccgctggcgggcactatctggaaggtgctggccagcgaaggccagacggtggccgcaggcgaggtgctgctgattctggaagccatgaagatggaaaccgaaatccgcgccgcgcaggccgggaccgtgcgcggtatcgcggtgaaagccggcgacgcggtggcggtcggcgacaccctgatgaccctggcgggctctggatccgatctgtacgacgatgacgataagggaattgatcccttcacc

SEQ ID NO: 28 - protein, 91 aa, Artificial
  pET104/D-TOPO vector
MGAGTPVTAPLAGTIWKVLASEGQTVAAGEVLLILEAMKMETEIRAAQAGTVRGIAVKAGDAVAVGDTLMTLAGSGSDLYDDDDKGIDPFT

SEQ ID NO: 29 - DNA, 450 bp, Artificial
  pcDNA/Biotag-DEST vector
cccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttaccatgggcgccggcaccccggtgaccgccccgctggcgggcactatctggaaggtgctggccagcgaaggccagacggtggccgcaggcgaggtgctgctgattctggaagccatgaagatggaaaccgaaatccgcgccgcgcaggccgggaccgtgcgcggtatcgcggtgaaagccggcgacgcggtggcggtcggcgacaccctgatgaccctggcgggctctggatccgatctgtacgacgatgacgataaggtacatcaaacaagtttgtacaaaaaagcaggctnn

SEQ ID NO: 30 - protein, 96 aa, Artificial
  pcDNA/Biotag-DEST vector
MGAGTPVTAPLAGTIWKVLASEGQTVAAGEVLLILEAMKMETEIRAAQAGTVRGIAVKAGDAVAVGDTLMTLAGSGSDLYDDDDKVHQTSLYKKAG

SEQ ID NO: 31 - DNA, 453 bp, Artificial
  pcDNA6/Biotag/D-TOPO
cccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttaccatgggcgccggcaccccggtgaccgccccgctggcgggcactatctggaaggtgctggccagcgaaggccagacggtggccgcaggcgaggtgctgctgattctggaagccatgaagatggaaaccgaaatccgcgccgcgcaggccgggaccgtgcgcggtatcgcggtgaaagccggcgacgcggtggcggtcggcgacaccctgatgaccctggcgggctctggatccgatctgtacgacgatgacgataaggtacctaggatccagtgtggtggaattgatcccttcacc

SEQ ID NO: 32 - protein, 98 aa, Artificial
  pcDNA6/Biotag/D-TOPO
MGAGTPVTAPLAGTIWKVLASEGQTVAAGEVLLILEAMKMETEIRAAQAGTVRGIAVKAGDAVAVGDTLMTLAGSGSDLYDDDDKVPRIQCGGIDPFT

SEQ ID NO: 33 - DNA, 744 bp, Artificial
  pMT/Biotag-DEST vector
cgttgcaggacaggatgtggtgcccgatgtgactagctctttgctgcaggccgtcctatcctctggttccgataagagacccagaactccggccccccaccgcccaccgccacccccatacatatgtggtacgcaagtaagagtgcctgcgcatgccccatgtgccccaccaagagttttgcatcccatacaagtccccaaagtggagaaccgaaccaattcttcgcgggcagaacaaaagcttctgcacacgtctccactcgaatttggagccggccggcgtgtgcaaaagaggtgaatcgaacgaaagacccgtgtgtaaagccgcgtttccaaaatgtataaaaccgagagcatctggccaatgtgcatcagttgtggtcagcagcaaaatcaagtgaatcatctcagtgcaactaaaggggggatctagcgtttaaacttaagcttaccatgggcgccggcaccccggtgaccgccccgctggcgggcactatctggaaggtgctggccagcgaaggccagacggtggccgcaggcgaggtgctgctgattctggaagccatgaagatggaaaccgaaatccgcgccgcgcaggccgggaccgtgcgcggtatcgcggtgaaagccggcgacgcggtggcggtcggcgacaccctgatgaccctggcgggctctggatccgatctgtacgacgatgacgataaggtacatcaaacaagtttgtacaaaaaagcaggctnn

SEQ ID NO: 34 - protein, 96 aa, Artificial; identical to SEQ ID NO: 30
  pMT/Biotag-DEST vector